(19) United States
(12) Patent Application Publication
(10) Pub. No.: US 2020/0095639 A1
(43) Pub. Date: Mar. 26, 2020

(54) GENETIC MARKERS ASSOCIATED WITH ASD AND OTHER CHILDHOOD DEVELOPMENTAL DELAY DISORDERS

(71) Applicant: LINEAGEN, INC., Salt Lake City, UT (US)

(72) Inventor: Charles H. HENSEL, Salt Lake City, UT (US)

(21) Appl. No.: 16/408,154

(22) Filed: May 9, 2019

Related U.S. Application Data
(63) Continuation of application No. 16/144,934, filed on Sep. 27, 2018, now abandoned.
Application No. 16/144,934 is a continuation of application No. 14/433,572, filed on Apr. 3, 2015, now abandoned.
(60) Provisional application No. 61/799,848, filed on Mar. 15, 2013.
(60) Provisional application No. 61/717,313, filed on Oct. 23, 2012.
(60) Provisional application No. 61/709,427, filed on Oct. 4, 2012.

(51) Int. Cl. C12Q 1/6883

(52) U.S. Cl. CPC C12Q 1/6883; C12Q 2600/156

(57) ABSTRACT

The present invention relates generally to genetic markers for autism spectrum disorders and other childhood developmental delay disorders, in particular to copy number variant genetic markers for autism spectrum disorders.

### CROSS-REFERENCE TO RELATED APPLICATIONS

This application is a continuation of U.S. application Ser. No. 16/144,934, filed Sep. 27, 2018, which is a continuation of U.S. application Ser. No. 14/433,572, filed Apr. 3, 2015, pending, which is a U.S. national phase application of International PCT Patent Application No. PCT/US2013/063532, filed on Oct. 4, 2013, which claims the benefit under 35 U.S.C. § 119(e) of U.S. Provisional Patent Application No. 61/799,848, filed Mar. 15, 2013, U.S. Provisional Patent Application No. 61/717,313, filed Oct. 23, 2012, and U.S. Provisional Patent Application No. 61/709,427, filed Oct. 4, 2012, each of which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is LINE_004_05US_ST25.txt. The text file is 12.2 MB, was created on May 7, 2019, and is being submitted electronically via EFS-Web.

### BACKGROUND

### Description of the Related Art

According to the National Institute of Mental Health (NIMH), autism is a group of developmental brain disorders, collectively referred to as autism spectrum disorder (ASD). As the term “spectrum” might suggest, ASD encompasses a wide range of symptoms, skills, and levels of impairment, or disability, that children with the disorder can have and is a complex, heterogeneous, behaviorally-defined disorder characterized by impairments in social interaction and communication as well as by repetitive and stereotyped behaviors and interests. The Diagnostic and Statistical Manual of Mental Disorders, Fourth Edition—Text Revision defines five disorders, sometimes called pervasive developmental disorders (PDDs), as ASD. These include: Autistic disorder (classic autism), Asperger's disorder (Asperger syndrome), Pervasive developmental disorder not otherwise specified (PDD-NOS), Rett's disorder (Rett syndrome), and Childhood disintegrative disorder (CDD). It is noted that the majority of Rett syndrome cases are known to be caused by mutations in either the MeCP2 gene or the CDKL5 gene and it is anticipated that updated revisions of the Diagnostic and Statistical Manual of Mental Disorders will classify Rett syndrome separately from ASD.

While environmental elements, such as peri- and post-natal stress, likely contribute to the development of ASD, evidence of chromosomal abnormalities, mutations in single genes, and multiple gene polymorphisms in autistic individuals show that autism is a genetic disorder.

Prevalence estimates for ASD have been reported to be approximately 1 in every 100 children in the general population. In families with an autistic child, the risk is estimated to be greater than 15% that an additional offspring will also have autism (Landa R J, Holman K C, Garrett-Mayer E. Social and communication development in toddlers with early and later diagnosis of autism spectrum disorders. Arch Gen Psychiatry 2007; 64:853-64; Landa R J. Diagnosis of autism spectrum disorders in the first 3 years of life. Nat Clin Pract Neurol 2008; 4:138-47).

The current state-of-the-art diagnosis of ASD is a series of various behavioral questionnaires. Because the ASD phenotype is so complicated, a molecular-based test would greatly improve the accuracy of diagnosis at an earlier age, when phenotypic/behavioral assessment is not possible, or integrated with phenotypic/behavioral assessment. Also, diagnosis at an earlier age would allow initiation of ASD treatment at an earlier age which may be beneficial to short and long-term outcomes.

Genetic factors play a substantial role in ASD (Abrahams B S, Geschwind D H. Advances in autism genetics: on the threshold of a new neurobiology. Nat Rev Genet 2008; 9:341-55). Previous genome-wide linkage and association studies have implicated multiple genetic regions that may be involved in autism and ASDs. Such heterogeneity increases the value of studies that include large extended pedigrees. Many autism studies have focused on small families (sibling pairs, or two parents and an affected offspring) to try to localize autism predisposition genes. These collections of small families may include cases with many different susceptibility loci. Subjects affected with ASD who are members of a large extended family may be more likely to share the same genetic causes through their common ancestors. Within such families, autism may be more genetically homogeneous.

While there is no known medical treatment for autism, some success has been reported for early intervention with behavioral therapies. Identification of biomarkers for ASD would allow identification of the disease, now typically diagnosed between ages three and five, in infancy or prenatal life. Thus, there is an urgent need for a method of reliably identifying subjects with ASD. In particular there is need for a more accurate test for polymorphisms causing autism spectrum disorders and other childhood developmental delay disorders. Families with affected members would benefit from knowing whether they carry a mutation which could affect future pregnancies. Clinicians need a test as an aid in diagnosis, and researchers would use the test to classify subjects according to the etiology of their disease. The present invention provides this and other advantages.

### BRIEF SUMMARY

One aspect of the present invention provides a diagnostic test for diagnosing or predicting ASD in a subject comprising: a reagent for detecting at least one CNV genetic marker associated with ASD, wherein the at least one CNV genetic marker associated with ASD comprises: at least one CNV genetic marker associated with ASD listed in Table 3; and 0 or more CNV genetic markers associated with ASD listed in Table 4; wherein detection in a genetic sample from the subject of the at least one CNV genetic marker associated with ASD indicates that the subject is affected with ASD, or is predisposed to ASD. In one embodiment of the diagnostic tests described herein, the at least one CNV genetic marker associated with ASD listed in Table 3 is selected from the group consisting of the CNV genetic markers associated with ASD 4-7, 9-12, 14-20 and 22-24 listed in Table 3. In another embodiment of the diagnostic tests described herein, the at least one CNV genetic marker associated with ASD listed in Table 3 is selected from the group consisting of the CNV genetic markers associated with ASD 1-20 and 22-24 listed in Table 3. In yet another embodiment of the diagnostic tests described herein, the at least one CNV genetic marker associated with ASD listed in Table 3 comprises one or more of the CNV genetic markers numbered 6, 8, 10, 16 and 22 in Table 3 and wherein the 0 or more CNV genetic markers associated with ASD listed in Table 4 comprises 0 or more of CNV genetic markers numbered 2-5, 8-10, 16, 20, 22, 24, 30 and 32 listed in Table 4. In a further embodiment of the diagnostic tests described herein, the at least one CNV genetic marker associated with ASD listed in Table 3 comprises one or more of the CNV genetic markers numbered 2, 8, 11-13, 21 and 24 listed in Table 3; and the 0 or more CNV genetic markers associated with ASD listed in Table 4 comprises 0 or more of CNV genetic markers numbered 4, 6, 7, 10, 18, 19, 21, 22, 23, 26, 29 and 30 listed in Table 4.

In one embodiment of the diagnostic tests described herein, the at least one CNV genetic marker associated with ASD comprises: at least 5 CNV genetic marker associated with ASD listed in Table 3; and at least 5 CNV genetic markers associated with ASD listed in Table 4. In another embodiment, the at least one CNV genetic marker associated with ASD comprises: at least 10 CNV genetic marker associated with ASD listed in Table 3; and at least 10 CNV genetic markers associated with ASD listed in Table 4. In certain embodiments, the at least one CNV genetic marker associated with ASD comprises: at least 20 CNV genetic marker associated with ASD listed in Table 3; and at least 20 CNV genetic markers associated with ASD listed in Table 4. In one embodiment, the diagnostic tests described herein comprises at least one CNV genetic marker associated with ASD, wherein the at least one CNV genetic marker associated with ASD comprises the CNV genetic markers associated with ASD listed in Table 3; and the CNV genetic markers associated with ASD listed in Table 4.

In one embodiment of the diagnostic tests described herein for diagnosing or predicting ASD in a subject, the ASD in the subject comprises autism, Asperger's disorder, pervasive developmental disorder not otherwise specified, or childhood disintegrative disorder.

In one embodiment of the diagnostic test for diagnosing or predicting ASD in a subject comprising a reagent for detecting at least one CNV genetic marker associated with ASD, the reagent for detecting comprises one or more sets of oligonucleotides, wherein each set of oligonucleotides specifically hybridizes to a CNV genetic marker associated with ASD. In one embodiment, the one or more sets of oligonucleotides each comprises from about 2 to about 30 oligonucleotides. In another embodiment, the one or more sets of oligonucleotides each comprises from about 10 to about 25 oligonucleotides. In certain embodiments, the one or more sets of oligonucleotides each comprises from about 15 to about 20 oligonucleotides, and in another embodiment the one or more sets of oligonucleotides each comprises about 20 oligonucleotides. In certain embodiments, the one or more sets of oligonucleotides are on an array which in certain embodiments may be a high density microarray.

In one embodiment of the diagnostic test for diagnosing or predicting ASD in a subject comprising a reagent for detecting at least one CNV genetic marker associated with ASD, the reagent for detecting comprises one or more sets of oligonucleotides, and in one embodiment the one or more sets of oligonucleotides comprise DNA probes. In one embodiment, the DNA probes are selected from the sequences set forth in SEQ ID NOs: 7410-7426; 12508-12563; 27988-28001; 31283-31314; 32494-32587; 33402-39860; 51803-52100; 61165-61290; 62966-62998; 64149-64167; 69319-69561. In certain embodiments, the one or more sets of oligonucleotides comprise amplification primers that amplify the CNV genetic marker associated with ASD.

In certain embodiments, the diagnostic tests of the present invention have a diagnostic yield for ASD of about 10% to about 12%.

Another aspect, the present invention provides a method of diagnosing or predicting ASD in a subject, comprising: detecting in a genetic sample isolated from the subject at least one CNV genetic marker associated with ASD, wherein the at least one CNV genetic marker associated with ASD comprises: at least one CNV genetic marker associated with ASD listed in Table 3; and 0 or more CNV genetic markers associated with ASD listed in Table 4; thereby diagnosing or predicting ASD in the subject. In one embodiment, the ASD in the subject comprises autism, Asperger's disorder, pervasive developmental disorder not otherwise specified, or childhood disintegrative disorder. In certain embodiments of the methods of diagnosing or predicting ASD in a subject, the at least one CNV genetic marker associated with ASD listed in Table is selected from the group consisting of the CNV genetic markers associated with ASD 1-20 and 22-24 listed in Table 3. In another embodiment, the at least one CNV genetic marker associated with ASD listed in Table 3 comprises one or more of the CNV genetic markers numbered 6, 8, 10, 16 and 22 in Table 3 and wherein the 0 or more CNV genetic markers associated with ASD listed in Table 4 comprises 0 or more of CNV genetic markers numbered 2-5, 8-10, 16, 20, 22, 24, 30 and 32 listed in Table 4. In a further embodiment, the at least one CNV genetic marker associated with ASD listed in Table 3 comprises one or more of the CNV genetic markers numbered 2, 8, 11-13, 21 and 24 listed in Table 3; and the 0 or more CNV genetic markers associated with ASD listed in Table 4 comprises 0 or more of the CNV genetic markers numbered 4, 6, 7, 10, 18, 19, 21, 22, 23, 26, 29 and 30 listed in Table 4.

In another embodiment of a method of diagnosing or predicting ASD in a subject which comprises detecting in a genetic sample isolated from the subject at least one CNV genetic marker associated with ASD, the at least one CNV genetic marker associated with ASD comprises: at least 5 CNV genetic marker associated with ASD listed in Table 3; and at least 5 CNV genetic markers associated with ASD listed in Table 4. In one embodiment, the at least one CNV genetic marker associated with ASD comprises: at least 10 CNV genetic marker associated with ASD listed in Table 3; and at least 10 CNV genetic markers associated with ASD listed in Table 4. In certain embodiments, the at least one CNV genetic marker associated with ASD comprises: at least 20 CNV genetic marker associated with ASD listed in Table 3; and at least 20 CNV genetic markers associated with ASD listed in Table 4. In another embodiment, the at least one CNV genetic marker associated with ASD comprises: the CNV genetic markers associated with ASD listed in Table 3; and the CNV genetic markers associated with ASD listed in Table 4.

In one embodiment of the methods of diagnosing or predicting ASD, the at least one CNV genetic marker associated with ASD is detected by hybridizing one or more sets of DNA probes to at least one CNV genetic marker associated with ASD using a microarray, which in certain embodiments comprises a glass, plastic, or silicon biochip microarray. In another embodiment the microarray comprises a bead array or a high density microarray. In yet another embodiment, the one or more sets of DNA probes on the microarray comprise DNA probes selected from the sequences set forth in SEQ ID NOs:1-83,443.

In one embodiment of the methods of diagnosing or predicting ASD, the at least one CNV genetic marker associated with ASD is detected by next-generation sequencing, and in another embodiment, the at least one CNV genetic marker associated with ASD is detected by amplifying one or more portions of the at least one CNV genetic marker associated with ASD using PCR.

Another aspect of the present invention provides a method of diagnosing or predicting ASD in a subject, comprising: hybridizing a genetic sample isolated from the subject with one or more sets of oligonucleotides, wherein each set of oligonucleotides specifically hybridizes to a CNV genetic marker associated with ASD; wherein the at least one CNV genetic marker associated with ASD comprises: at least one CNV genetic marker associated with ASD listed in Table 3; and 0 or more CNV genetic markers associated with ASD listed in Table 4; thereby diagnosing or predicting ASD in the subject. In one embodiment of the methods herein, the ASD in the subject comprises autism, Asperger's disorder, pervasive developmental disorder not otherwise specified, Rett's disorder, or childhood disintegrative disorder. In another embodiment of the methods of diagnosing or predicting ASD in a subject, the at least one CNV genetic marker associated with ASD listed in Table 3 comprises one or more of the CNV genetic markers numbered 6, 8, 10, 16 and 22 in Table 3 and wherein the 0 or more CNV genetic markers associated with ASD listed in Table 4 comprises 0 or more of CNV genetic markers numbered 2-5, 8-10, 16, 20, 22, 24, 30 and 32 listed in Table 4. In another embodiment, the at least one CNV genetic marker associated with ASD listed in Table 3 comprises one or more of the CNV genetic markers numbered 2, 8, 11-13, 21 and 24 listed in Table 3; and the 0 or more CNV genetic markers associated with ASD listed in Table 4 comprises 0 or more of the CNV genetic markers numbered 4, 6, 7, 10, 18, 19, 21, 22, 23, 26, 29 and 30 listed in Table 4.

In another embodiment of the methods of diagnosing or predicting ASD in a subject which comprises hybridizing a genetic sample isolated from the subject with one or more sets of oligonucleotides, wherein each set of oligonucleotides specifically hybridizes to a CNV genetic marker associated with ASD, the at least one CNV genetic marker associated with ASD comprises: at least 5 CNV genetic marker associated with ASD listed in Table 3; and at least 5 CNV genetic markers associated with ASD listed in Table 4. In certain embodiments, the at least one CNV genetic marker associated with ASD comprises: at least 10 CNV genetic marker associated with ASD listed in Table 3; and at least 10 CNV genetic markers associated with ASD listed in Table 4. In a further embodiment, the at least one CNV genetic marker associated with ASD comprises: at least 20 CNV genetic marker associated with ASD listed in Table 3; and at least 20 CNV genetic markers associated with ASD listed in Table 4. In another embodiment, the at least one CNV genetic marker associated with ASD comprises: the CNV genetic markers associated with ASD listed in Table 3; and the CNV genetic markers associated with ASD listed in Table 4.

In another embodiment of the methods of diagnosing or predicting ASD in a subject which comprises hybridizing a genetic sample isolated from the subject with one or more sets of oligonucleotides, wherein each set of oligonucleotides specifically hybridizes to a CNV genetic marker associated with ASD, the one or more sets of oligonucleotides each comprises from about 2 to about 30 oligonucleotides. In another embodiment, the one or more sets of oligonucleotides each comprises from about 10 to about 25 oligonucleotides. In a further embodiment, the one or more sets of oligonucleotides each comprises from about 15 to about 20 oligonucleotides. In certain embodiments, the one or more sets of oligonucleotides comprise DNA probes arrayed on a microarray. In this regard, the DNA probes arrayed on a microarray may comprise DNA probes selected from the sequences set forth in SEQ ID NOs:1-83,443. In one embodiment, the one or more sets of oligonucleotides comprise amplification primers that amplify the CNV genetic marker associated with ASD.

Another aspect of the present invention provides a DNA microarray for detecting the presence of a CNV associated with ASD in a subject comprising one or more of the DNA probe sets selected from those set forth in SEQ ID NOs: 7410-7426; 12508-12563; 27988-28001; 31283-31314; 32494-32587; 33402-39860; 51803-52100; 61165-61290; 62966-62998; 64149-64167; 69319-69561. As would be recognized by the skilled person, such a microarray may also include additional DNA probes, such as commercially available DNA probes (e.g., such as those available from Illumina or the Affymetrix CytoScan-HD array). In another embodiment, the DNA microarray comprises at least 100 DNA probes selected from the DNA probes set forth in SEQ ID NOs: 7410-7426; 12508-12563; 27988-28001; 31283-31314; 32494-32587; 33402-39860; 51803-52100; 61165-61290; 62966-62998; 64149-64167; 69319-69561. In a further embodiment, the DNA microarray comprises at least 1000, at least 10000, at least 15000, at least 20000, or at least 50000 DNA probes selected from the DNA probes set forth in SEQ ID NOs: 1-83,443.

Another aspect of the present invention provides a method for determining the genotype of an individual suspected of having an ASD or other childhood developmental delay disorder, comprising hybridizing a genetic sample isolated from the subject with one or more sets of DNA probes, wherein the one or more sets of DNA probes are selected from the DNA probes set forth in SEQ ID NOs: 1-83,443. Childhood developmental delay disorders include but are not limited to Rett syndrome, Noonan/Costello/CFC syndromes, Tuberous sclerosis, ADHD, developmental delay (DD), Tourette syndrome, and Dyslexia.

Another aspect of the present invention provides a diagnostic test for diagnosing or predicting ASD in a subject comprising: a reagent for detecting at least one CNV genetic marker associated with ASD, wherein the at least one CNV genetic marker associated with ASD comprises: at least one CNV genetic marker associated with ASD listed in Table 8; or at least one CNV genetic marker associated with ASD listed in Table 10; or both; and 0 or more CNV genetic markers associated with ASD listed in Table 9; wherein detection in a genetic sample from the subject of the at least one CNV genetic marker associated with ASD indicates that the subject is affected with ASD, or is predisposed to ASD.

### BRIEF DESCRIPTION OF THE DRAWINGS

FIG. 1: Workflow for CNV analysis for samples analyzed on the custom array. The same process was used for both CNAM and PennCNV analyses. All samples used for CNV analysis in this study had to meet the quality control measures described. Only unrelated cases and controls were used for the final statistical analysis.

FIG. 2: Manhattan plot of CNVs called both by PennCNV and CNAM. Association statistics across all regions covered on the Illumina custom array are shown. Since the array used was not a genome-wide array, the width of each chromosome on the plot is not proportional to the chromosome length. Adjacent chromosomes are separated by tick marks.

FIG. 3. UCSC Genome browser view of CNVs in the NRXN1 region. CNVs observed in the vicinity of the NRXN1-alpha transcription start site are shown. Note that most CNVs observed in ASD patients include exon 1 of NRXN1-alpha while only 1 control CNV extends into exon 1. Produced with custom tracks listing CNV calls and uploaded to the genome.ucsc.edu website.

FIG. 4. UCSC Genome Browser View of CNVs in the GABR Region on chromosome 15q12. Duplications were called by both PennCNV and by CNAM in this region, however the number of duplications called by each program differed, with many additional duplications called by CNAM. Produced with custom tracks listing CNV calls and uploaded to the genome.ucsc.edu website.

### DETAILED DESCRIPTION

The present invention relates generally to genetic markers for ASD, in particular to copy number variant genetic markers for ASD. In particular, the present CNV genetic markers associated with ASD provide a diagnostic yield (the percentage of individuals with the diagnosis of ASD that will have an abnormal genetic test result; equal to sensitivity) of about 10-12%, while generic chromosomal microarray technologies currently available are expected to remain in the 5%-7% diagnostic yield range for the autism-specific portion of these microarrays (that is, 5-7% of the individuals with ASD that are tested with current technologies will have an abnormal result). Thus, the present invention represents a 2× increase (5% to more than 10%) in autism—specific diagnostic yield over current diagnostic platforms.

The practice of the present invention will employ, unless indicated specifically to the contrary, conventional methods of microbiology, molecular biology, recombinant DNA technique, chemical syntheses, chemical analyses, pharmaceutical preparation, formulation, and delivery, and treatment of patients, within the skill of the art, many of which are described below for the purpose of illustration. Such techniques are explained fully in the literature. See, e.g., Current Protocols in Protein Science, Current Protocols in Molecular Biology or Current Protocols in Immunology, John Wiley & Sons, New York, N.Y. (2009); Ausubel et al., Short Protocols in Molecular Biology, 3rd ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

As used in this specification and the appended claims, the singular forms “a,” “an” and “the” include plural references unless the content clearly dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Each embodiment in this specification is to be applied mutatis mutandis to every other embodiment unless expressly stated otherwise.

The Diagnostic and Statistical Manual of Mental Disorders, Fourth Edition—Text Revision currently defines five disorders, sometimes called pervasive developmental disorders (PDDs), as ASD. These include: Autistic disorder (classic autism), Asperger's disorder (Asperger syndrome (AS)), Pervasive developmental disorder not otherwise specified (PDD-NOS), Rett's disorder (Rett syndrome), and Childhood disintegrative disorder (CDD). It is noted that the majority of Rett syndrome cases are known to be caused by mutations in either the MeCP2 gene or the CDKL5 gene and it is anticipated that updated revisions of the Diagnostic and Statistical Manual of Mental Disorders will classify Rett syndrome separately from ASD. Therefore, in certain embodiments, ASD does not include Rett syndrome. Autism shall be understood as any condition of impaired social interaction and communication with restricted repetitive and stereotyped patterns of behavior, interests and activities present before the age of 3, to the extent that health may be impaired. AS is distinguished from autistic disorder by the lack of a clinically significant delay in language development in the presence of the impaired social interaction and restricted repetitive behaviors, interests, and activities that characterize ASD. PDD-NOS is used to categorize individuals who do not meet the strict criteria for autism but who come close, either by manifesting atypical autism or by nearly meeting the diagnostic criteria in two or three of the key areas.

Developmental delay disorders are an ever growing group of disorders. Any chromosomal deletion or duplication that results in symptoms such as hypotonia (muscle weakness), intellectual disability, dysmorphic physical features, repetitive behaviors, etc. is included under the umbrella of developmental delay conditions that can be detected using the present invention. Specific examples include, but are not limited to, chromosome 22q13.3 deletion syndrome, Prader-Willi syndrome and Angelman syndrome, and chromosome 1p36 deletion syndrome, just to name a few. Childhood developmental delay disorders may also include, but are not limited to, Rett syndrome, Noonan/Costello/CFC syndromes, Tuberous sclerosis, ADHD, developmental delay (DD), Tourette syndrome, and Dyslexia. The OMIM web site (internet address can be found at ncbi.nlm.nih.gov/omim) keeps an updated list of disorders and a description of the specific genotype identified, that can be accessed by the skilled person.

There are also a host of disorders that are associated with autism (Autism-associated disorders). These diseases or pathologies include, more specifically, any metabolic and immune disorders, epilepsy, anxiety, depression, attention deficit hyperactivity disorder, speech delay or language impairment, motor incoordination, mental retardation, schizophrenia and bipolar disorder. The various embodiments and examples disclosed herein may be used in various subjects, particularly human, including adults and children and at the prenatal stage.

As used herein, the term “subject” means any target of administration. The subject can be a vertebrate, for example, a mammal. Thus, the subject can be a human. The term does not denote a particular age or sex. Thus, adult and newborn subjects, as well as fetuses, whether male or female, are intended to be covered. A patient refers to a subject afflicted with a disease or disorder. Unless otherwise specified, the term “patient” includes human and veterinary subjects.

As used herein, the term “biomarker” or “biological marker” means an indicator of a biologic state and may include a characteristic that is objectively measured as an indicator of normal biological processes, pathologic processes, or pharmacologic responses to a therapeutic or other intervention. In one embodiment, a biomarker may indicate a change in expression or state of a protein that correlates with the risk or progression of a disease, or with the susceptibility of the disease in an individual. In certain embodiments, a biomarker may include one or more of the following: genes, proteins, glycoproteins, metabolites, cytokines, and antibodies.

The present invention centers on the discovery and validation of copy number variant (CNV) genetic markers associated with ASD. SNPs are known to be the primary source of human genetic variation. However, structural variations, including copy number variations (e.g., relatively large regions of the genome that have been deleted or duplicated on certain chromosomes), also contribute to genetic and phenotypic human variation (see e.g., Feuk, et al., 2006 Nature Reviews Genetics, 7, 85-97).

A CNV represents a copy number change involving a DNA fragment that is about 1 kilobases (kb) or larger (see e.g., Feuk, et al., 2006 Nature Reviews Genetics, 7, 85-97). CNVs described herein do not include those variants that arise from the insertion/deletion of transposable elements (e.g., .about.6-kb Kpnl repeats) to minimize the complexity of CNV analyses. The term CNV therefore encompasses previously introduced terms such as large-scale copy number variants (LCVs; lafrate et al. 2004 Nat Genet. 36:949-951), copy number polymorphisms (CNPs; Sebat et al. 2004 Science. 305:525-528), and intermediate-sized variants (ISVs; Tuzun et al. 2005 Nat Genet. 37:727-732), but not retroposon insertions.

A single nucleotide polymorphism (SNP) refers to a change in which a single base in the DNA differs from the usual base at that position. These single base changes are called SNPs or “snips.” Millions of SNPs have been cataloged in the human genome. Some SNPs such as that which causes sickle cell are responsible for disease. Other SNPs are normal variations in the genome.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, in one embodiment, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to single stranded nucleic acids, particularly oligonucleotides, the term “specifically hybridize” refers to the association between two single-stranded nucleotide molecules of sufficient complementary sequence to permit such hybridization under pre-determined conditions generally used in the art (sometimes termed “substantially complementary”). In particular, in one embodiment the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence. For example, specific hybridization can refer to a sequence which hybridizes to an ASD associated marker gene or nucleic acid (e.g., the CNV genetic markers associated with ASD as described herein), but does not hybridize to other nucleotides. Appropriate conditions enabling specific hybridization of single stranded nucleic acid molecules of varying complementarity are well known in the art.

The term “genetic marker” as used herein refers to one or more inherited or de novo variations in DNA structure with a known physical location on a chromosome. Genetic markers include variations, or polymorphisms, in specific nucleotides or chromosome regions. Examples of genetic markers include, single nucleotide polymorphisms (SNPs), and copy number variations and copy number changes (CNVs). Genetic markers can be used to associate an inherited phenotype, such as a disease, with a responsible genotype. Genetic markers may be used to track the inheritance of a nearby gene that has not yet been identified, but whose approximate location is known. The genetic marker itself may be a part of a gene's coding region or regulatory region. For example, a genetic marker may be a functional polymorphism that may alter gene function or gene expression. Alternatively, a genetic marker may be a non-functional polymorphism.

A CNV genetic marker refers to a DNA sequence having a copy number variation, with a known location on a chromosome, which can be used to identify individuals, in particular subjects affected by or at risk of developing ASD. The CNV genetic markers associated with ASD described herein, were identified in an extensive replication/refinement study of CNV markers. In particular, a custom array was designed and used to genotype about 3000 individuals with autism and 6000 individuals with normal development. A combination of 2 different statistical and bioinformatics algorithms was used to make the CNV calls and proved to be highly accurate. In particular, 97% of the CNVs called using the combination of algorithms were subsequently validated by other laboratory methods, as compared to 30% using only the individual algorithms (see Example 1). The CNV genetic markers associated with ASD identified herein are provided in Tables 3 and 4. The CNV genetic markers shown in Tables 3 and 4 are those CNV genetic markers having an odds ratio (the likelihood that a given genetic marker is relevant to a diagnosis of ASD in an individual) of 2 or higher.

While certain of the CNV genetic markers associated with ASD shown in Table 4 overlap with previously identified CNV genetic markers, the markers had not been previously extensively refined and validated until the present study. Therefore, the present disclosure provides newly identified CNV genetic markers as well as refined and validated genetic markers, that greatly improve the diagnostic yield of ASD diagnostic tests over what was previously known. Thus, the present disclosure provides a more diagnostically comprehensive and accurate set of CNV genetic markers associated with ASD that can be used in the diagnosis of ASD. Illustrative DNA probes that can be used to genotype individuals for the presence of CNVs associated with ASD are provided in the sequence listing which includes SEQ ID NOs:1-83,433. These DNA probes also include custom probes to genotype other childhood developmental delay disorders, including for example, Rett syndrome, Noonan/Costello/CFC syndromes, Tuberous sclerosis, ADHD, DD, and Tourette syndrome. Particularly illustrative DNA probes for detecting the presence of CNVs associated with ASD are provided in SEQ ID NOs: 7410-7426; 12508-12563; 27988-28001; 31283-31314; 32494-32587; 33402-39860; 51803-52100; 61165-61290; 62966-62998; 64149-64167; 69319-69561.

The CNV genetic markers associated with ASD as described herein are generally defined by their chromosomal location and are referred to by the most recent human genome coordinates (e.g., hg19 chromosomal location coordinates). However, as would be understood by the skilled artisan, as the exact region of the CNV (e.g., the region of highest significance) is further characterized and refined, the CNV region boundaries may shift to the left or to the right while getting smaller, or may get smaller within the same region as originally defined. For example, the CNVs listed in Table 3 are referred to by the CNV region as defined in the discovery cohort as well as the CNV region as defined in the replication cohort. As shown in Table 3, the CNV region for the first listed marker has been reduced from the region spanning chr1:145714421-146101228 to the region spanning chr1: 145703115-145736438, with the left boundary shifting further to the left. The region boundaries for CNV marker number 6 listed in Table 3 have shifted to the right and have been reduced. Therefore, as would be understood by the skilled person, the CNV markers associated with ASD as described herein comprise the CNV region as described herein and include the surrounding region to the left and to the right of the CNV chromosomal region as described herein. Thus, in certain embodiments, the chromosomal region encompassing the CNV genetic markers associated with ASD described herein may comprise the chromosomal region 50, 100, 200, 300, 400, 500, 600, 700, 800, 900, 1000, 1500, 2000, 2500, 3000, 3500, 4000, 4500, 5000, 6000, 7000, 8000, 9000, 10000, 15,000, 20000, 30000, 40000, 50,000, 60,000, 70,000, 80,000, 90,000, 100,000, or more positions to the left and/or to the right of the chromosomal region as described herein.

Further, in related embodiments, reagents for detecting the CNV genetic markers as described herein include reagents which specifically hybridize to the chromosomal regions surrounding the region specifically described herein. In particular, a nucleic acid reagent for detecting the CNV genetic markers as described herein may specifically hybridize to the chromosomal region 50, 100, 200, 300, 400, 500, 600, 700, 800, 900, 1000, 1500, 2000, 2500, 3000, 3500, 4000, 4500, 5000, 6000, 7000, 8000, 9000, 10000, 15,000, 20000, 30000, 40000, 50,000, 60,000, 70,000, 80,000, 90,000, 100,000, or more positions to the left and/or to the right of the chromosomal region of the CNV genetic marker as described herein.

In certain embodiments, genes in or adjacent to the CNV genetic markers may also be detected using detection reagents in the tests and methods for diagnosing or predicting ASD described herein. In this regard, such genes include but are not limited to NRXN1, LINGO2, STXBP5, GABA receptor gene cluster (e.g., GABRA5, GABRA3, GABRG3), RGS20, TCEA1, UBE3A, E2F1, PLCB1, PMP22, AADAT, MAPK3, NRXN1, NRG3, DPP10, UQCRC2, USH2A, NECAB3, CNTN4, LINGO2, IL1RAPL1, STXBP5, DOC2A, SNRPN, CDRT15, CDH13, CD160, CALCR, and SPN. Further genes contemplated for use in the tests and methods described herein include those listed in Tables 3, 4, 8 and 10. Reagents for detecting such genes may detect the DNA, RNA expression, protein activity or downstream biological functions of the protein encoded by such genes in or adjacent to the CNV genetic markers described herein. Thus, the present invention includes reagents for detecting such genes or the expression thereof, including nucleic acids, DNA probes, antibodies that bind to the encoded proteins, and the like.

In one embodiment, the detection of the presence of a genetic marker or functional polymorphism associated with a gene linked to ASD may indicate that the subject is affected with ASD or is at risk of developing ASD. A subject who is at increased risk of developing ASD is one who is predisposed to the disease, has genetic susceptibility for the disease and/or is more likely to develop the disease than subjects in which the genetic marker is present or is absent.

In one embodiment, the presence of one or more CNV genetic markers described herein indicates that an individual is affected with ASD or is predisposed to developing ASD (e.g., predisposed to developing autism, Asperger Disorder, PDD-NOS, or Childhood disintegrative disorder (CDD). In another embodiment, the presence of one or more CNV genetic markers described herein may be predictive of whether an individual is at risk for or susceptible to ASD. If certain genetic polymorphisms (e.g., CNVs) are detected more frequently in people with ASD, the variations are said to be “associated” with ASD. In this regard, variations may be associated with autism, asperger disorder or PDD-NOS, Rett's disorder (Rett syndrome), CDD, or a combination thereof. The polymorphisms associated with ASD may either directly cause the disease phenotype or they may be in linkage disequilibrium (LD) with nearby genetic mutations that influence the individual variation in the disease phenotype. As used herein, LD is the nonrandom association of alleles at 2 or more loci.

Accordingly, the present invention relates to diagnostic tests for diagnosing or predicting ASDs in subjects. In this regard, the present invention relates to diagnostic tests for diagnosing or predicting autism, asperger disorder and/or PDD-NOS in subjects. The diagnostic tests described herein may be in vitro diagnostic tests. Diagnostic tests include but are not limited to FDA approved, or cleared, In Vitro Diagnostic (IVD), Laboratory Developed Test (LDT), or Direct-to-Consumer (DTC) tests, that may be used to assay a sample and detect or indicate the presence of, the predisposition to, or the risk of, diseases, disorders, conditions, infections and/or therapeutic responses. In one embodiment, a diagnostic test may be used in a laboratory or other health professional setting. In another embodiment, a diagnostic test may be used by a consumer at home. Diagnostic tests comprise one or more reagents for detecting the CNV genetic markers associated with ASD as described herein and may comprise other reagents, instruments, and systems intended for use in the in vitro diagnosis of disease or other conditions, including a determination of the state of health, in order to cure, mitigate, treat, or prevent disease or its sequelae. In one embodiment, the diagnostic tests described herein may be intended for use in the collection, preparation, and examination of specimens taken from the human body. In certain embodiments, diagnostic tests and products may comprise one or more laboratory tests. As used herein, the term “laboratory test” means one or more medical or laboratory procedures that involve testing samples of blood, urine, or other tissues or substances in the body.

The diagnostic tests of the present invention comprise one or more reagents for detecting the CNV genetic markers associated with ASD as described herein, such as those provided in Tables 3 and 4. In this regard, the reagents for detecting may comprise any reagent known to the skilled person for detecting genetic markers.

Illustrative reagents for detecting genetic markers include nucleic acids, and in particular include oligonucleotides. A nucleic acid can be DNA or RNA, and may be single or double stranded. In one embodiment, the oligonucleotides are DNA probes, or primers for amplifying nucleic acids of genetic markers. In one embodiment, the oligonucleotides of the present invention are capable of specifically hybridizing (e.g, under stringent hybridization conditions), with complementary regions of a genetic marker associated with ASD containing a genetic polymorphism described herein, such as a copy number variation. Oligonucleotides can be naturally occurring or synthetic, but are typically prepared by synthetic means. Oligonucleotides, as described herein, may include segments of DNA, or their complements. The exact size of the oligonucleotide will depend on various factors and on the particular application and use of the oligonucleotide. Oligonucleotides, which include probes and primers, can be any length from 3 nucleotides to the full length of a target nucleic acid molecule of interest (e.g., a nucleic acid molecule of a CNV genetic marker associated with autism spectrum disorders, such as those provided in the tables herein), and explicitly include every possible number of contiguous nucleic acids from 3 through the full length of a target polynucleotide of interest. Thus, oligonucleotides can be between 5 and 100 contiguous bases, and often range from 5, 10, 12, 13, 14, 15, 16, 17, 18, 19, 20, 21, 22, 23, 24, or 25 nucleotides to 30, 35, 40, 45, 50, 55, 60, 65, 70, 75, 80, 85, 90, 95, or 100 nucleotides. Oligonucleotides between 5-10, 5-20, 10-20, 12-30, 15-30, 10-50, 20-50 or 20-100 bases in length are common.

Oligonucleotides of the present invention can be RNA, DNA, or derivatives of either. The minimum size of such oligonucleotides is the size required for formation of a stable hybrid between an oligonucleotide and a complementary sequence on a nucleic acid molecule of the present invention (i.e., the copy number variant genetic markers described herein). The present invention includes oligonucleotides that can be used as, for example, probes to identify nucleic acid molecules (e.g., DNA probes) or primers to amplify nucleic acid molecules.

In one embodiment, an oligonucleotide may be a probe which refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. In certain embodiments, a probe can be between 5 and 100 contiguous bases, and is generally about 5, 10, 12, 13, 14, 15, 16, 17, 18, 19, 20, 21, 22, 23, 24, or 25 nucleotides in length, or may be about 30, 35, 40, 45, 50, 55, 60, 65, 70, 75, 80, 85, 90, 95, or 100 nucleotides in length. The probes herein are selected to be complementary to different strands of a particular target nucleic acid sequence. This means that the probes must be sufficiently complementary so as to be able to specifically hybridize or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically. Illustrative probes for detecting the genetic markers associated with ASD and other childhood developmental delay disorders are set forth in SEQ ID NOs:1-83,443. In particular, DNA probes for detecting CNVs associated with ASD are set forth in SEQ ID NOs: 7410-7426; 12508-12563; 27988-28001; 31283-31314; 32494-32587; 33402-39860; 51803-52100; 61165-61290; 62966-62998; 64149-64167; 69319-69561. (See also Table 11 for a description of the childhood developmental delay disorders and the custom DNA probes provided in the sequence listing and Table 14). As would be recognized by the skilled person, a specific probe or probe set disclosed herein for detecting a particular CNV associated with ASD (or other disorder), can be identified by using the hg19 chromosomal location start and end coordinates of a CNV of interest (e.g., a CNV listed in Table 3 or 4) to query Table 14 to find a corresponding overlapping chromosomal location in Table 14. Those probes that are listed in Table 14 for the overlapping hg19 chromosomal location are those probes that can be used to detect the particular CNV. Note that Table 14 discloses illustrative probes and does not include probes for all CNVs associated with ASD described herein. Additional probes may be designed by the skilled person using known techniques.

In one embodiment, an oligonucleotide may be a primer, which refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in certain applications, an oligonucleotide primer is about 15-25 or more nucleotides in length, but may in certain embodiments be between 5 and 100 contiguous bases, and often be about 5, 10, 12, 13, 14, 15, 16, 17, 18, 19, 20, 21, 22, 23, 24, or 25 nucleotides long or, in certain embodiments, may be about 30, 35, 40, 45, 50, 55, 60, 65, 70, 75, 80, 85, 90, 95, or 100 nucleotides in length for. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Thus, in one embodiment, a CNV genetic marker associated with ASD as described herein may be detected by amplification of the region of interest according to amplification protocols well known in the art (e.g., polymerase chain reaction, 2D cluster PCR amplification (see, e.g., Illumina, Inc., San Diego, Calif.), ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA)). The amplification product can then be visualized directly in a gel by staining, the product can be detected by hybridization with a detectable probe, and/or by using next generation sequencing. When amplification conditions allow for amplification of all allelic types of a genetic marker, the types can be distinguished by a variety of well-known methods, such as, but not limited to, hybridization with an allele-specific probe, secondary amplification with allele-specific primers, restriction endonuclease digestion, or electrophoresis. Thus, the present invention can further provide oligonucleotides for use as primers and/or probes for detecting and/or identifying genetic markers according to the methods of this invention.

“Sample” or “patient sample” or “biological sample” generally refers to a sample which may be tested for a particular molecule, preferably a CNV genetic marker associated with ASD, such as a marker shown in the tables provided herein. Samples may include but are not limited to cells, buccal swab sample, body fluids, including blood, serum, plasma, urine, saliva, cerebral spinal fluid, tears, pleural fluid and the like.

In certain embodiments, a reagent for detecting the CNV genetic markers associated with ASD comprises one or more sets of oligonucleotides, wherein each set of oligonucleotides specifically hybridizes to a CNV genetic marker associated with ASD. As used herein a set of oligonucleotides may comprise from about 2 to about 100 oligonucleotides, all of which specifically hybridize to a particular CNV genetic marker associated with ASD. In one embodiment, a set of oligonucleotides comprises from about 5 to about 30 oligonucleotides, from about 10 to about 20 oligonucleotides, and in one embodiment comprises about 20 oligonucleotides, all of which specifically hybridize to a particular CNV genetic marker associated with ASD. Thus, a set of oligonucleotides may comprise about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200 or more oligonucleotides, all of which specifically hybridize to a particular CNV genetic marker associated with ASD. In one embodiment, a set of oligonucleotides comprises DNA probes. In one embodiment, the DNA probes comprise overlapping DNA probes. In another embodiment, the DNA probes comprise nonoverlapping DNA probes. In one embodiment, the DNA probes provide detection coverage over the length of a CNV genetic marker associated with ASD. In another embodiment, a set of oligonucleotides comprises amplification primers that amplify a CNV genetic marker associated with ASD. In this regard, sets of oligonucleotides comprising amplification primers may comprise multiplex amplification primers. In another embodiment, the sets of oligonucleotides or DNA probes may be provided on an array, such as solid phase arrays, chromosomal/DNA microarrays, or micro-bead arrays. Array technology is well known in the art. Illustrative arrays contemplated for use in the present invention include, but are not limited to, arrays available from Affymetrix (Santa Clara, Calif.) and Illumina (San Diego, Calif.).

In one embodiment, an array comprises one or more DNA probes or sets of probes as set forth in SEQ ID NOs:1-83,443. In one embodiment, an array comprises at least 1, 2, 3, 4, 5, 6, 7, 8, 9, 10, 11, 12, 13, 14, 15, 16, 17, 18, 19, 20, or more DNA probes as set forth in SEQ ID NOs:1-83,443. In another embodiment, an array for identifying the genotype of a subject suspected of having ASD or other childhood developmental delay disorder, comprises at least about 25-2500, or at least 100, 1000, 10000,15000, 16000, 17000, 18000, 19000, 20000, 25000, 30000, 35000, 40000, 45000, 50000, 55000, 60000, 65000 or more of the DNA probes forth in SEQ ID NOs:1-83,443. In another embodiment, an array for genotyping an individual for the presence of a CNV associated with ASD or other childhood developmental delay disorder, comprises the DNA probes set forth in the sequence listing and identified in Table 14 that are custom probes for the CNVs listed in Tables 8 and 9, which specifically hybridize to the CNVs identified in Table 3 and 4. In one embodiment, an array for genotyping an individual for the presence of a CNV associated with ASD, comprises the DNA probes set forth in SEQ ID NOs: 7410-7426; 12508-12563; 27988-28001; 31283-31314; 32494-32587; 33402-39860; 51803-52100; 61165-61290; 62966-62998; 64149-64167; 69319-69561.

As generally known in the art, a variety of arrays can be used for detection of polymorphisms that can be correlated to the phenotypes of interest. In one embodiment, DNA probe array chips or larger DNA probe array wafers (from which individual chips would otherwise be obtained by breaking up the wafer) may be used. In one such embodiment, DNA probe array wafers may comprise glass wafers on which high density arrays of DNA probes (short segments of DNA) have been placed. Each of these wafers can hold, for example, millions of DNA probes that are used to recognize sample DNA sequences (e.g., from individuals or populations that may comprise polymorphisms of interest). The recognition of sample DNA by the set of DNA probes on the glass wafer takes place through DNA hybridization. When a DNA sample hybridizes with an array of DNA probes, the sample binds to those probes that are complementary to the sample DNA sequence. By evaluating to which probes the sample DNA for an individual hybridizes more strongly, it is possible to determine whether a known sequence of nucleic acid is present or not in the sample, thereby determining whether a polymorphism found in the nucleic acid is present.

In one embodiment, the use of DNA probe arrays to obtain allele information typically involves the following general steps: design and manufacture of DNA probe arrays, preparation of the sample, hybridization of sample DNA to the array, detection of hybridization events, and data analysis to determine sequence. In one such embodiment, wafers may be manufactured using a process adapted from semiconductor manufacturing to achieve cost effectiveness and high quality, and are available, e.g., from Affymetrix, Inc. of Santa Clara, Calif.

Arrays of interest may further comprise sequences, including polymorphisms, of other genetic sequences, particularly other sequences of interest for pharmacogenetic screening and a variety of control sequences. As with other human polymorphisms, the polymorphisms of the invention also have more general applications, such as forensic, paternity testing, linkage analysis and positional cloning.

In certain embodiments, the oligonucleotides for detecting CNV genetic markers associated with ASD may be used in high throughput sequencing methods (often referred to as next-generation sequencing methods or next-gen sequencing methods). Accordingly, in one embodiment, the present disclosure provides methods of diagnosing or predicting ASD in a subject by detecting in a genetic sample from the subject at least one CNV genetic marker associated with ASD as described herein, wherein the at least one CNV genetic marker associated with ASD is detected by high throughput sequencing. High throughput sequencing, or next-generation sequencing, methods are known in the art (see e.g., Zhang et al., J Genet Genomics. 2011 Mar. 20; 38(3):95-109; Metzker, Nat Rev Genet. 2010 January; 11(1):31-46) and include, but are not limited to, technologies such as ABI SOLiD sequencing technology (now owned by Life Technologies, Carlsbad, Calif.); Roche 454 FLX which uses sequencing by synthesis technology known as pyrosequencing (Roche, Basel Switzerland); Illumina Genome Analyzer (Illumina, San Diego, Calif.); Dover Systems Polonator G.007 (Salem, N.H.); Helicos (Helicos BioSciences Corporation, Cambridge Mass., USA), and Sanger. In one embodiment, DNA sequencing may be performed using methods well known in the art including mass spectrometry technology and whole genome sequencing technologies (e.g. those used by Pacific Biosciences, Menlo Park, Calif., USA), etc.

In another embodiment, the presence of or the absence of one or more genetic markers may be visualized by staining or marking the genetic markers with molecular dyes, probes, or other analytes and reagents specific to the genetic markers of interest. In one such embodiment, the genetic markers may be detected by automated methods comprising fluorescent probes, melting curve analysis, and other genetic marker detection methods known by those of skill in the art. In one embodiment, one or more genetic markers may be detected and the detected genetic markers may be visualized on a display showing the location of the genetic markers on a genetic sample. In one such embodiment, the detection of one or more genetic markers may be detected by an electronic device which generates a signal that may be shown on a display in order for a user to visualize the presence of or the absence of one or more genetic markers, and/or the location of one or more genetic markers.

In various embodiments, the oligonucleotides for detecting the CNV genetic markers associated with ASD described herein are conjugated to a detectable label that may be detected directly or indirectly. In the present invention, DNA probes, RNA probes, monoclonal antibodies, antigen-binding fragments thereof, and antibody derivatives thereof, may all be covalently linked to a detectable label.

A “detectable label” is a molecule or material that can produce a detectable (such as visually, electronically or otherwise) signal that indicates the presence and/or concentration of the label in a sample. When conjugated to a nucleic acid such as a DNA probe, the detectable label can be used to locate and/or quantify a target nucleic acid sequence to which the specific probe is directed. Thereby, the presence and/or amount of the target in a sample can be detected by detecting the signal produced by the detectable label. A detectable label can be detected directly or indirectly, and several different detectable labels conjugated to different probes can be used in combination to detect one or more targets.

Examples of detectable labels, which may be detected directly, include fluorescent dyes and radioactive substances and metal particles. In contrast, indirect detection requires the application of one or more additional probes or antibodies, i.e., secondary antibodies, after application of the primary probe or antibody. Thus, in certain embodiments, as would be understood by the skilled artisan, the detection is performed by the detection of the binding of the secondary probe or binding agent to the primary detectable probe. Examples of primary detectable binding agents or probes requiring addition of a secondary binding agent or antibody include enzymatic detectable binding agents and hapten detectable binding agents or antibodies.

In some embodiments, the detectable label is conjugated to a nucleic acid polymer which comprises the first binding agent (e.g., in an ISH, WISH, or FISH process). In other embodiments, the detectable label is conjugated to an antibody which comprises the first binding agent (e.g., in an IHC process).

Examples of detectable labels which may be conjugated to the oligonucleotides used in the methods of the present disclosure include fluorescent labels, enzyme labels, radioisotopes, chemiluminescent labels, electrochemiluminescent labels, bioluminescent labels, polymers, polymer particles, metal particles, haptens, and dyes.

Examples of fluorescent labels include 5-(and 6)-carboxyfluorescein, 5- or 6-carboxyfluorescein, 6-(fluorescein)-5-(and 6)-carboxamido hexanoic acid, fluorescein isothiocyanate, rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, green fluorescent protein (GFP) and analogues thereof, and conjugates of R-phycoerythrin or allophycoerythrin, inorganic fluorescent labels such as particles based on semiconductor material like coated CdSe nanocrystallites.

Examples of polymer particle labels include micro particles or latex particles of polystyrene, PMMA or silica, which can be embedded with fluorescent dyes, or polymer micelles or capsules which contain dyes, enzymes or substrates.

Examples of metal particle labels include gold particles and coated gold particles, which can be converted by silver stains. Examples of haptens include DNP, fluorescein isothiocyanate (FITC), biotin, and digoxigenin. Examples of enzymatic labels include horseradish peroxidase (HRP), alkaline phosphatase (ALP or AP), β-galactosidase (GAL), glucose-6-phosphate dehydrogenase, β-N-acetylglucosamimidase, β-glucuronidase, invertase, Xanthine Oxidase, firefly luciferase and glucose oxidase (GO). Examples of commonly used substrates for horseradishperoxidase include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), .alpha.-naphtol pyronin (.alpha.-NP), o-dianisidine (OD), 5-bromo-4-chloro-3-indolylphosp-hate (BCIP), Nitro blue tetrazolium (NBT), 2-(p-iodophenyl)-3-p-nitropheny-I-5-phenyl tetrazolium chloride (INT), tetranitro blue tetrazolium (TNBT), 5-bromo-4-chloro-3-indoxyl-beta-D-galactoside/ferro-ferricyanide (BCIG/FF).

Examples of commonly used substrates for Alkaline Phosphatase include Naphthol-AS-B 1-phosphate/fast red TR (NABP/FR), Naphthol-AS-MX-phosphate/fast red TR (NAMP/FR), Naphthol-AS-B1-phosphate/-fast red TR (NABP/FR), Naphthol-AS-MX-phosphate/fast red TR (NAMP/FR), Naphthol-AS-B1-phosphate/new fuschin (NABP/NF), bromochloroindolyl phosphate/nitroblue tetrazolium (BCIP/NBT), 5-Bromo-4-chloro-3-indolyl-b-d-galactopyranoside (BCIG).

Examples of luminescent labels include luminol, isoluminol, acridinium esters, 1,2-dioxetanes and pyridopyridazines. Examples of electrochemiluminescent labels include ruthenium derivatives. Examples of radioactive labels include radioactive isotopes of iodide, cobalt, selenium, tritium, carbon, sulfur and phosphorous.

Detectable labels may be linked to any molecule that specifically binds to a biological marker of interest, e.g., an antibody, a nucleic acid probe, or a polymer. Furthermore, one of ordinary skill in the art would appreciate that detectable labels can also be conjugated to second, and/or third, and/or fourth, and/or fifth binding agents, nucleic acids, or antibodies, etc. Moreover, the skilled artisan would appreciate that each additional binding agent or nucleic acid used to characterize a biological marker of interest (e.g., the CNV genetic markers associated with ASD) may serve as a signal amplification step. The biological marker may be detected visually using, e.g., light microscopy, fluorescent microscopy, electron microscopy where the detectable substance is for example a dye, a colloidal gold particle, a luminescent reagent. Visually detectable substances bound to a biological marker may also be detected using a spectrophotometer. Where the detectable substance is a radioactive isotope detection can be visually by autoradiography, or non-visually using a scintillation counter. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.).

One aspect of the present invention comprises a diagnostic test for diagnosing or predicting ASD in an individual comprising a reagent for detecting at least one CNV genetic marker associated with ASD, wherein the at least one CNV genetic marker associated with ASD comprises: at least one CNV genetic marker associated with ASD listed in Table 3; and 0 or more CNV genetic markers associated with ASD listed in Table 4; wherein detection in a genetic sample from the individual of the at least one CNV genetic marker associated with ASD indicates that the individual is affected with ASD, or is predisposed to ASD. In one embodiment, the at least one CNV genetic marker associated with ASD listed in Table 3 is selected from the group consisting of the CNV genetic markers associated with ASD 1-20 and 22-24 listed in Table 3. In one embodiment the at least one CNV genetic marker associated with ASD listed in Table 3 comprises one or more of the CNV genetic markers numbered 6, 8, 10, 16 and 22 in Table 3 and wherein the 0 or more CNV genetic markers associated with ASD listed in Table 4 comprises 0 or more of CNV genetic markers numbered 2-5, 8-10, 16, 20, 22, 24, 30 and 32 listed in Table 4. In another embodiment, the at least one CNV genetic marker associated with ASD listed in Table 3 comprises one or more of the CNV genetic markers comprising a gene in or adjacent to said CNV genetic marker that is involved in neural function, development and disease, such as one or more of the CNV genetic markers numbered 2, 8, 11-13, 21 and 24 listed in Table 3; and the 0 or more CNV genetic markers associated with ASD listed in Table 4 comprises 0 or more of CNV genetic markers numbered 4, 6, 7, 10, 18, 19, 21, 22, 23, 26, 29 and 30 listed in Table 4 (e.g., CNV genetic markers comprising a gene in or adjacent to it that is involved in neural function, development and disease). In another embodiment, the at least one CNV genetic marker associated with ASD comprises at least 5, 6, 7, 8, 9, 10, 11, 12, 13, 14, 15, 16, 17, 18, 19, 20, 21, 22, 23 or all 24 of the CNV genetic markers associated with ASD listed in Table 3; and at least 5, 6, 7, 8, 9, 10, 11, 12, 13, 14, 15, 16, 17, 18, 19, 20, 25, 30 or all the CNV genetic markers associated with ASD listed in Table 4. In one embodiment, a diagnostic test for diagnosing or predicting ASD in a subject comprises a reagent for detecting at least one CNV genetic marker associated with ASD, wherein the at least one CNV genetic marker associated with ASD comprises at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 90, 100, or more of the CNV genetic markers associated with ASD listed in Table 8; and at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 90, 100, or more of the CNV genetic markers associated with ASD listed in Table 9. In one embodiment, a diagnostic test for diagnosing or predicting ASD in a subject comprises a reagent for detecting at least one CNV genetic marker associated with ASD, wherein the at least one CNV genetic marker associated with ASD comprises all the CNV genetic markers associated with ASD listed in Table 3; and all the CNV genetic markers associated with ASD listed in Table 4.

In one embodiment, a diagnostic test as described herein has a diagnostic yield for ASD of about 8% to about 40%. Diagnostic yield refers to the percent of individuals with the diagnosis of ASD that will have an abnormal genetic test result and is equal to sensitivity. In this regard, the diagnostic test described herein may have a diagnostic yield for ASD of about 8% to about 14%, from about 9% to about 13%, or from about 10% to about 12%. In further embodiments, a diagnostic test as described herein has a diagnostic yield for ASD of about 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or about 40%.

In certain embodiments, the CNV genetic markers associated with ASD as described herein may be isolated, amplified, and/or cloned into a vector. The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention (e.g., an isolated nucleic acid containing a CNV associated with ASD as described herein) can be inserted into a vector by cutting the vector with restriction enzymes and ligating the two pieces together.

Many techniques are available to those skilled in the art to facilitate transformation, transfection, or transduction of an expression construct into a prokaryotic or eukaryotic organism. The terms “transformation”, “transfection”, and “transduction” refer to methods of inserting a nucleic acid and/or expression construct into a cell or host organism. These methods involve a variety of techniques known to the skilled artisan, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of a nuclic acid molecule of a genetic marker associated with ASD such that the latter is transcribed into mRNA. Host cell machinery then translates mRNA into a polypeptide.

Those skilled in the art will recognize that a nucleic acid vector can contain nucleic acid elements other than the promoter element and the autism specific marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

In one embodiment, the methods and in vitro diagnostic tests and products described herein may be used for the diagnosis of autism in at-risk patients, patients with non-specific symptoms possibly associated with autism, and/or patients presenting with related disorders (e.g., asperger, disorder, PDD-NOS, and CDD). In another embodiment, the methods and in vitro diagnostic tests described herein may be used for screening for risk of progressing from at-risk, non-specific symptoms possibly associated with ASD, and/or fully-diagnosed ASD. In certain embodiments, the methods and in vitro diagnostic tests described herein can be used to rule out screening of diseases and disorders that share symptoms with ASD. In yet another embodiment, the methods and in vitro diagnostic tests described herein may indicate diagnostic information to be included in the current diagnostic evaluation in patients suspected of having autism and other related disorder classified under ASD.

In one embodiment, a diagnostic test may comprise one or more devices, tools, and equipment configured to collect a genetic sample from an individual. In one embodiment of a diagnostic test, tools to collect a genetic sample may include one or more of a swab, a scalpel, a syringe, a scraper, a container, and other devices and reagents designed to facilitate the collection, storage, and transport of a genetic sample. In one embodiment, a diagnostic test may include reagents or solutions for collecting, stabilizing, storing, and processing a genetic sample. Such reagents and solutions for collecting, stabilizing, storing, and processing genetic material are well known by those of skill in the art. In another embodiment, a diagnostic test as disclosed herein, may comprise a microarray apparatus and associated reagents, a flow cell apparatus and associated reagents, a multiplex next generation nucleic acid sequencer and associated reagents, and additional hardware and software necessary to assay a genetic sample for the presence of certain genetic markers and to detect and visualize certain genetic markers.

In certain embodiments, the methods disclosed herein may comprise assaying the presence of one or more CNV genetic markers in an individual which may include methods generally known in the art. In one such embodiment, methods for detecting a genetic polymorphism such as a CNV genetic marker associated with ASD in an individual may include assaying an individual for the presence of or the absence of a CNV associated with ASD using one or more genotyping assays such as an array, PCR-based genotyping, next-generation sequencing-based methods, DNA hybridization, fluorescence microscopy, and other methods known by those of skill in the art. In another embodiment, methods for assaying the presence of or the absence of one or more CNV markers associated with ASD may include providing a nucleotide sample from an individual and assaying the nucleotide sample for the presence of or the absence of one or more CNV genetic markers. In one embodiment, the sample may be a biological fluid or tissue comprising nucleated cells including genomic material.

Described herein are methods for detecting the risk, diagnosing, and predicting ASD in an individual by detecting one or more CNV genetic markers associated with ASD. In one embodiment, the methods disclosed herein may be used to indicate if an individual is at risk of developing ASD. In one embodiment, the methods disclosed herein may be used to diagnose ASD in an individual. In one embodiment, the methods disclosed may be used to characterize the clinical course or status of ASD in a subject. In one embodiment, the methods as disclosed herein may be used to predict a response in a subject to an existing treatment for ASD, or a treatment for ASD that is in development or has yet to be developed. The methods described herein can be employed to screen for any type of disorder associated with autism, including, any metabolic and immune disorders, epilepsy, anxiety, depression, attention deficit hyperactivity disorder, speech delay or language impairment, motor incoordination, mental retardation, schizophrenia and bipolar disorder.

In certain embodiments, one or more CNV genetic markers described herein can be used in a method for selecting a patient for treatment of an ASD. For example, the presence or absence of the CNV genetic marker indicates that the patient will, e.g., be responsive to and/or benefit (e.g., reduce one or more symptoms of the ASD) from the treatment. In one embodiment, a patient may be selected for a particular treatment if the patient comprises a CNV genetic marker provided in Tables 3, 4, 8 and 9. In another embodiment, a patient that does not comprise a CNV genetic marker selected from the genetic markers provided in Tables 3, 4, 8 and 9 is selected for a particular treatment.

In one embodiment, a method of selecting a patient for treatment comprises detecting a CNV genetic marker associated with ASD. In certain embodiments, the CNV genetic marker is associated with at least one of autism, Asperger's disorder, PDD-NOS, Rett's disorder, and CDD.

In one embodiment, the patient is selected for the treatment of classic autism. Treatments include, e.g., gene therapy, RNA interference (RNAi), behavioral therapy (e.g., Applied Behavior Analysis (ABA), Discrete Trial Training (DTT), Early Intensive Behavioral Intervention (EIBI), Pivotal Response Training (PRT), Verbal Behavior Intervention (VBI), and Developmental Individual Differences Relationship-Based Approach (DIR)), physical therapy, occupational therapy, sensory integration therapy, speech therapy, the Picture Exchange Communication System (PECS), dietary treatment, and drugs (e.g., antipsychotics, anti-depressants, anticonvulsants, stimulants).

In another embodiment, the patient is selected for the treatment of Asperger's disorder. Treatments include, e.g., gene therapy, RNAi, occupational therapy, physical therapy, communication and social skills training, cognitive behavioral therapy, speech or language therapy, and drugs (e.g., aripiprazole, guanfacine, selective serotonin reuptake inhibitors (SSRIs), riseridone, olanzapine, naltrexone).

In one embodiment, the patient is selected for the treatment of Rett's disorder. Treatments include, e.g., gene therapy, RNAi, occupational therapy, physical therapy, speech or language therapy, nutritional supplements, and drugs (e.g., SSRIs, anti-psychotics, beta-blockers, anticonvulsants).

In one embodiment, the patient is selected for the treatment of CDD.

Treatments include, e.g., gene therapy, RNAi, behavioral therapy (e.g., ABA, DTT, EIBI, PRT, VBI, and DIR), sensory enrichment therapy, occupational therapy, physical therapy, speech or language therapy, nutritional supplements, and drugs (e.g., anti-psychotics and anticonvulsants).

In another embodiment, the patient is selected for the treatment of PDD-NOS. Treatments include, e.g., gene therapy, RNAi, behavioral therapy (e.g., ABA, DTT, EIBI, PRT, VBI, and DIR), physical therapy, occupational therapy, sensory integration therapy, speech therapy, PECS, dietary treatment, and drugs (e.g., antipsychotics, anti-depressants, anticonvulsants, stimulants).

In one embodiment, the treatment the patient is selected for is gene therapy to correct, replace, or compensate for a target gene. Gene therapy may target an overexpressed gene or an underexpressed gene. In one embodiment, a patient comprises a CNV genetic marker in or adjacent to a gene to be modified by gene therapy. Examples of genes in or adjacent to the CNV genetic markers described herein include, but are not limited to, NRXN1, LINGO2, STXBP5, GABA receptor gene cluster (e.g., GABRA5, GABRA3, GABRG3), RGS20, TCEA1, UBE3A, E2F1, PLCB1, PMP22, AADAT, MAPK3, NRXN1, NRG3, DPP10, UQCRC2, USH2A, NECAB3, CNTN4, LINGO2, IL1RAPL1, STXBP5, DOC2A, SNRPN, CDRT15, CDH13, CD160, CALCR, and SPN. Examples of other genes that may be targeted by gene therapy include MECP2, CDKL5 and FOXG1.

### EXAMPLES

### Example 1

### Identification of Rare Recurrent Copy Number Variants in High-Risk Autism Families and Their Prevalence in a Large ASD Population

Genetics are known to play a major role in individuals with autism. However, the genetic underpinnings of autism are highly complex. The study described in this example used high-risk autism families to identify genetic variants that could predispose to autism in these families. This study also further evaluated these variants in a very large group of unrelated autism samples and controls to determine if these variants were relevant to children with autism in the broader population. This study identified 18 genetic variants that have not previously been observed in children with autism that are important not only in families but also in unrelated children with autism. By using a very large group of samples and controls this study also provides better frequency and significance estimates for many genetic variants previously associated with autism. This study sets the stage for using these genetic variants in the clinical analysis of children with autism.

Structural variation is thought to play a major etiological role in the development of ASDs, and numerous studies documenting the relevance of copy number variants in ASDs have been published since 2006. To determine if large ASD families harbor high-impact CNVs that may have broader impact in the general ASD population, the present experiments used the Affymetrix genome wide human SNP array 6.0 to identify 153 putative autism-specific CNVs present in 55 individuals with ASD from 9 multiplex ASD pedigrees. To evaluate the actual prevalence of these CNVs as well as 185 CNVs reportedly associated with ASD from published studies many of which are insufficiently powered, a custom Illumina array was designed and used to interrogate these CNVs in 3,000 ASD cases and 6,000 controls. Additional single nucleotide variants (SNVs) on the array identified 25 CNVs not detected in the family studies at the standard SNP array resolution. After molecular validation, the results demonstrated that 15 CNVs identified in high-risk ASD families also were found in two or more ASD cases with odds ratios greater than 2.0, strengthening their support as ASD risk variants. In addition, of the 25 CNVs identified using SNV probes on the custom array, 9 also had odds ratios greater than 2.0, suggesting that these CNVs also are ASD risk variants. Eighteen of the validated CNVs have not been reported previously in individuals with ASD and three have only been observed once. Finally, the results described here confirmed the association of 31 of 185 published ASD-associated CNVs in this dataset with odds ratios greater than 2.0, suggesting they may be of clinical relevance in the evaluation of children with ASDs. Taken together, these data provide strong support for the existence and application of high-impact CNVs in the clinical genetic evaluation of children with ASD.

### Introduction

Twin studies [1-3], (reviewed in [4]), family studies [5-7], and reports of chromosomal aberrations in individuals with ASD (reviewed in[8]) all have strongly suggested a role for genes in the development of ASD. Although the magnitude of the genetic effect observed in ASD varies from study to study, it is clear that genetics plays a significant role.

While a number of genes associated with ASD susceptibility have been observed in multiple studies, variants in a single gene cannot explain more than a small percentage of cases. Indeed, recent estimates suggest that there may be nearly 400 genes or chromosomal regions involved in ASD predisposition [9-12].

In the past few years, a number of studies have identified both de novo and inherited structural variants, CNVs, that are associated with ASD [13-23]. De novo CNVs may explain at least some of the “missing heritability” of ASD as understood to date. While it is clear that CNVs play an important role in susceptibility to ASD, it is also clear that the genetic penetrance of many of these CNVs is less than 100%. Although many of the duplications or deletions observed in children with ASD occur as de novo variants, duplications, for example on chromosome 16p11.2, often are inherited from an asymptomatic parent. Moreover, both deletions and duplications encompassing a portion of chromosome 16p11.2 have been associated with ASD [21,24-26] and 16p11.2 gains have been associated with ADHD and schizophrenia [24,27-29], indicating that the same genomic region can be involved in multiple developmental conditions. In addition, deletions on chromosome 7q11.23 are known to cause Williams syndrome and duplications of this same region have been observed and are thought to be causal in individuals with ASD [9,11]. While individuals with Williams syndrome tend to be outgoing and social, individuals with ASD are socially withdrawn, suggesting that deletions and duplications in this region result in individuals on opposite sides of the behavioral spectrum.

Although numerous studies regarding the role of CNVs in ASD have been published in the research literature, the findings of these studies have not been fully utilized for clinical evaluation of children with ASD. This is likely due to the rarity of individual variants, the lack of probe coverage on clinical microarrays that permits detection of smaller variants, and the difficulty in understanding the relevant biology of some variants even when they are significantly associated with ASD. Despite this, published clinical guidelines suggest that microarray-based testing should be the first step in the genetic analysis of children with syndromic and non-syndromic ASD as well as other conditions of childhood development [30], and there is a wealth of information demonstrating its utility in large samples of children who have undergone such testing [25,31].

This example describes efforts to discover high-impact CNVs in high-risk ASD families in Utah and to assess their potential role in unrelated ASD cases. These CNVs were interrogated, as well as CNVs from multiple published sources [18,32] in a large sample set of ASD cases and controls, to determine more precisely their potential disease relevance. To evaluate carefully these CNVs, a custom Illumina iSelect array was designed containing probes within and flanking CNV regions of interest. This custom array was used to obtain high-quality CNV results on 2,175 children with clinically diagnosed ASD and 5,801 children with normal development following removal of samples that did not meet stringent quality control parameters. The results of this study identify multiple rare recurrent CNVs from high-risk ASD families that also confer risk in unrelated ASD cases and delineate the prevalence and impact of CNVs reported in the literature in a large case control study of ASDs.

### Materials and Methods

DNA samples. DNA samples from high-risk ASD family members were collected after obtaining informed consent using a University of Utah IRB-approved protocol. Three independent sample cohorts, comprising 3,000 ASD patient samples (72% male), were collected for CNV replication. Of those, 857 were probands recruited and genotyped by the Center for Applied Genomics (CAG) at The Children's Hospital of Philadelphia (CHOP) from the greater Philadelphia area using a CHOP IRB-approved protocol; 2,143 ASD samples were from the AGRE and the AGP consortium (Rutgers, N.J. ASD repository), and genotyped at the CAG center at CHOP (Table 1). Only samples from affected individuals diagnosed using the Autism Diagnostic Interview-Revised (ADI-R) and the Autism Diagnostic Observation Schedule (ADOS) were used in the study. All control samples were from CHOP and were matched in a 2:1 ratio with the ASD cases.

TABLE 1
Case and control samples used in this study.

| | |
|---|---|
| case | control |

| | male | female | male | female |
|---|---|---|---|---|
| AGRE/AGP | 1,517 | 626 | 0 | 0 |
| CHOP | 633 | 224 | 3,992 | 2,008 |
| sub-total | 2,150 | 850 | 3,992 | 2,008 |

| | | |
|---|---|---|
| grand-total | 3,000 | 6,000 |

CNV Discovery in high-risk ASD families. DNA samples were genotyped on the Affymetrix Genome-Wide Human SNP Array 6.0 according to the manufacturer's protocol. Fifty-five autism subjects were chosen from 9 families with multiple affected first-degree relatives. The number of individuals with an autism diagnosis in these families ranged from 3 to 9. Affected individuals were diagnosed using ADI-R and ADOS. Control subjects (N=439) for the discovery phase of the project were selected from Utah CEPH/Genetics Reference Project (UGRP) families [70]. All microarray experiments were performed on blood DNA samples, except for two of the 55 case samples and three control subjects for which DNA from lymphoblastoid cell lines was used. CNVs were initially detected using the Copy Number Analysis Module (CNAM) of Golden Helix SNP & Variation Suite (SVS) (Golden Helix Inc.). Log ratios were calculated by quantile normalizing the A allele and B allele intensities using the entire population as a reference median for each SNP.

Batch effects in the log ratios were corrected via numeric principle component analysis (PCA) [71]. CNV segmentation analysis was carried out for each individual using the univariate CNAM segmentation procedure of Golden Helix SVS. We used a moving window of 5,000 markers, maximum number of segments per window of 20, minimum segment size 10 markers, and pairwise permutation p-value of 0.001.

iSelect array design. Probes for each CNV to be characterized in this study were selected from the Illumina Omni2.5 array probe set. Probes were selected to be as uniformly spaced across each region and flanking each region as possible (using the hg19 genome build). For each CNV, we included 10 or more probes within the defined CNV region (CNVr) and five probes on each flank (except where not possible due to the telomeric location of a CNVr). Probes for an additional 185 CNVs described in the literature, including 104 identified by CHOP in samples that partially overlap those used in this study, also were included for further CNV validation. We attempted to increase probe coverage for CNVs identified with only a small number of probes. Probes for 2,799 putative functional candidate SNVs detected by targeted exome DNA sequencing on 26 representative individuals from 11 ASD families (unpublished data) were included. The genes that were targeted for exome sequencing included all known genes in regions of familial haplotype sharing and linkage as well as additional autism candidate genes. These SNVs, although included in a search for potential ASD point mutations, also were used to identify additional CNVs.

Array processing. High throughput SNP genotyping using the Illumina Infinium™ II BeadChip technology (Illumina, San Diego), at the Center for Applied Genomics at CHOP was performed. Detailed methods for array processing are described in the section entitled Supplemental Materials below.

CNV calling and statistical analysis. CNVs were called using both PennCNV [34,35] and CNAM (Golden Helix SNP & Variation Suite (SVS), Golden Helix, Inc.). CNV calling using PennCNV was performed as described [32]. For CNAM calls, each target region was separately analyzed, rather than whole chromosomes. Since our array targeted specific regions and did not have probe coverage over much of the genome, it was desirable to avoid calling segments that spanned large regions with no data, and prevent any CNV calls from being influenced by distant data points. To accomplish this, the markers in the data set were grouped into “pseudochromosomes”, one for each CNV covered by the array, that were then considered individually in the segmentation algorithm. After segmentation, segments were classified as losses, gains, or neutral. Fisher's exact test was used to test for association of copy number loss versus no loss, and copy number gain versus no gain. Similar tests were conducted for the X chromosome, stratified by gender. Odds ratios also were calculated as an indicator of potential clinical risk for each CNV.

Laboratory confirmation of CNVs. Array results were confirmed using pre-designed Applied Biosystems TaqMan copy number assays or custom-designed TaqMan copy number assays when necessary (Life Technologies, Inc.). All CNVs with odds ratios greater than 2.0 and present in at least two cases were selected for molecular validation. We did not select CNVs with odds ratios less than 2 were not selected for validation because these odds ratios were not thought to have high potential clinical utility. Six CNVs were also selected for validation because they were adjacent to, but not overlapping, literature CNVs that were covered by probes on the custom array. A maximum of 6 case samples were validated for each CNV. Five negative control samples, selected based on their lack of all of the CNVs under study also were included in each validation assay. A list of all of the TaqMan assays used in this work is found in Table 7, and detailed procedures of the TaqMan assays are described in the supplemental methods.

Pathway analysis. Analysis of biological pathways encompassing genes found in the CNV regions was performed using the bioinformatics tools DAVID Bioinformatics Resources 6.7 [72,73] and Ingenuity Pathways Analysis (IPA) (Ingenuity® Systems). Network and pathway analyses on genes contained within the CNVs or immediately flanking intergenic CNVs that were PCR validated was performed. Pathway analysis details are described in the supplemental methods.

### Results

CNV discovery in Utah high risk autism pedigrees. Using CNAM (GoldenHelix Inc.) on Affymetrix Genome-Wide Human SNP array 6.0 data, a total of 153 CNVs in subjects with autism in Utah families that were not found in any CEPH/UGRP control samples were identified. This set included 131 novel CNVs and 22 CNVs present in the Autism Chromosomal Rearrangement Database [15]. Thirty-two autism-specific CNVs were detected in multiple (2 or more) autism subjects, and 121 CNVs were detected in only one person among the 55 autism subjects assayed. Of these, 153 CNVs, 112 were copy number losses (deletions) and 41 were copy number gains (duplications). The average size of the CNVs from high-risk families was 91 kb. The genomic locations of these CNVs are shown in Table 8.

CNV regions on the custom array. To better understand the frequency of the CNVs identified in Utah ASD families in a broader ASD population, we created a custom Illumina iSelect array containing probes covering all 153 of the Utah CNVs described in Table 8. CNV coordinate, copy number status, and probe content for each CNV are included. In addition, since the ultimate goal of this work is to understand the frequency and relevance of rare recurrent CNVs in the etiology of ASD, we included probes for 185 autism-associated CNVs identified in the literature [14-16,18,21,32,33] (Table 9). The probe coverage for each literature CNV also is shown in Table 9. In total, 7134 probes, all selected from the Illumina 2.5M array, were used for this study. As part of a separate study we also included 2799 SNVs detected by next-generation sequencing of genes in regions of haplotype sharing among our high-risk ASD families and in published ASD candidate genes in these same individuals also were included. Intensity data for these SNVs were used to identify additional CNVs that were not observed in our Utah high-risk ASD families (Table 10). Following standard data QC steps (see supplemental results) this array was used to characterize which of these 363 CNVs were present in DNA from 2,175 children with autism and 5,801 age, gender, and ethnicity matched controls (Table 1). These 7976 samples were available for analysis following our strict quality control measures (supplemental methods).

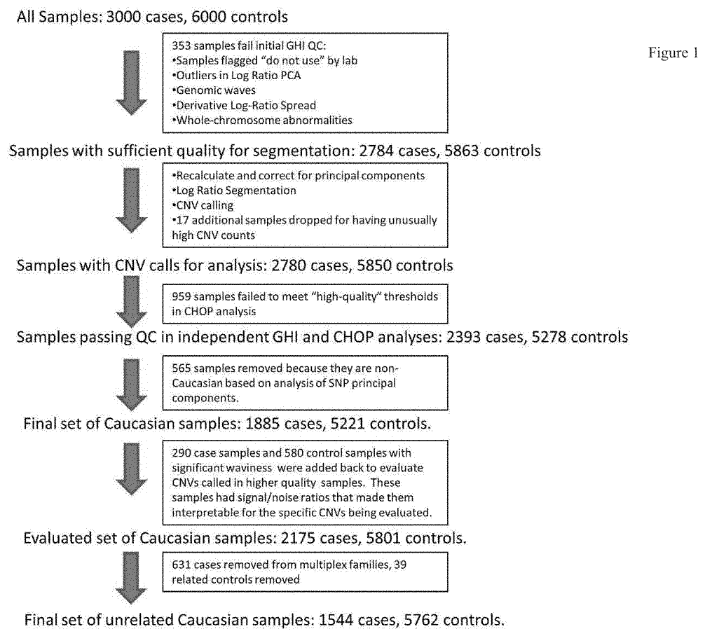

Analysis of CNVs on the iSelect array. The workflow for CNV analyzis of the custom array data is shown in FIG. 1. Following quality control analysis, including removal of samples that did not meet laboratory sample quality control measures, samples with excessive CNV calls, samples of uncertain ethnicity, and related samples, our final dataset included 1544 unrelated cases and 5762 unrelated controls. Because of the inherent noisiness of CNV analysis, we used two independent CNV calling algorithms, PennCNV [34] and CNAM (Golden Helix, Inc.), to increase our ability to detect CNVs. We identified 6,086 CNVs in cases and 14,387 CNVs in controls using PennCNV and 3,226 CNVs in cases and 8,234 CNVs in controls using CNAM. 1,537 CNVs from the 2175 cases including those from multiplex families (average 0.70 CNVs per individual) and 3,845 CNVs from the 5801 controls including related controls (average of 0.66 CNVs per individual) were called by both algorithms used for CNV detection.

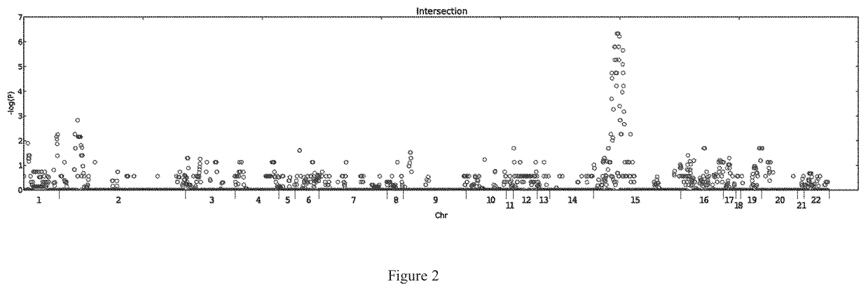

All CNV regions harboring CNVs shared among subjects were defined from PennCNV calls, CNAM calls and the PennCNV/CNAM intersecting calls and their significance of association was calculated across the genome (FIG. 2). Of the 153 CNVs discovered in high-risk ASD families, 139 of them were seen in replication samples evaluated with the custom Illumina iSelect array. Seven of the CNVs not seen in this larger population study had poor probe coverage on the array either due to their small size or their genomic content, while the remainder that were not detected may represent false positive CNVs from our initial discovery work or may be rare CNVs that are private to the families or individuals in which they were identified.

Molecular validation of CNV calls. We used TaqMan copy number assays to confirm the presence of CNVs in our population. A summary of the 195 TaqMan assays used is shown in Table 7 (Hs assay names refer to assays available from Applied Biosystems, now Life Technologies, Carlsbad, Calif.). Since our goal for this study was to understand the frequencies of these CNVs in a large case/control population, we chose to validate any CNVs that were likely to have clinical relevance. Our criteria for selection were as follows: 1) any CNV with an odds ratio >=2.0; 2) any rare CNV seen in at least two cases. These criteria for selecting CNVs were chosen to validate because the goal was to translate research CNV findings into potentially clinically useful markers. Since clinical testing of individuals with ASD is only performed on people who are symptomatic, CNVs with odds ratios <1.0 (CNVs that indicate lower than average risk of ASD) were not chosen for validation. Likewise, since CNVs with odds ratios >=1 but <=2 do are not of great diagnostic interest, we chose to validate only CNVs with odds ratios >=2.0. By using these criteria, we included rare recurrent CNVs that may be etiologically important despite the lack of statistical significance in cases versus controls. For previously published CNVs we considered our custom Illumina iSelect array as an independent test of their validity. We assumed therefore that these CNVs did not require additional testing. Since some of the CNVs from CHOP were not included in previous publications [18,32], we selected all CHOP CNVs for molecular validation. For CNVs that met our selection criteria we assayed a maximum of six case samples that contained the CNV, giving priority to those samples called both by PennCNV and CNAM. Results of these TaqMan experiments are summarized in Table 2. Interestingly, many of the most common CNVs detected by the array were not validated by the TaqMan assays. For example, when we tested samples from a statistically significant CNV duplication on chromosome 7q36.1 that was detected only by PennCNV and not by CNAM, all samples tested were shown to have two copies rather than the anticipated three copies, suggesting that in this sample set at least some of the CNV duplications observed are not true positives. Conversely all but one of the CNVs observed on chromosome 15, whether in the Prader-Willi/Angelman syndrome region or located more distally on chromosome 15, were confirmed by TaqMan assays. Results of these validation experiments demonstrated that CNVs called both by PennCNV and CNAM were much more likely to be confirmed (97% of tested samples) than CNVs called by either PennCNV alone (24%) or CNAM alone (30%). This observation demonstrates the care that must be taken during the CNV discovery process to insure that only valid calls are selected for further analysis.

False negative results also are possible with these microarray studies. However, the controls used for TaqMan assays were selected from the control sample set because they lacked CNV calls for any of the regions being evaluated. In none of these samples did the TaqMan results indicate the presence of any of the CNVs being validated, so no false negative results were detected. These data suggest that false negative results are not a common problem in this study.

TABLE 2
confirmation of CNV calls by quantitative PCR.

| TaqMan CNV | Utah Family | Utah Sequence | Literature | |
|---|---|---|---|---|
| Validation Status | CNVs | SNP CNVs | CNVs | Total |
| PASS | 24 (2 overlap | 15 | 25 | 64 |
| | with Lit. CNV) | | | |
| FAIL | 9 | 9 | 5 | 23 |
| NoCall | 0 | 1 | 0 | 1 |

A summary of the PCR validation result is shown. Sequence SNP CNVs were discovered in this work using SNVs present on this array for sequence variant confirmation in the same cohort.

CNVs from high-risk Utah families. One hundred thirty-nine of the 153 CNVs identified in high-risk ASD families were observed in case and/or control samples in this large dataset. Of these, 33 were present in two or more cases and had odds ratios greater than 2 and thus were selected for molecular confirmation. Following TaqMan validation, fifteen of thirty-three CNVs were confirmed (Table 3). This set included 3 CNVs with mixed results (Table 3). A CNV that was validated in some samples but not in others was considered to have passed validation if the validated samples resulted in an odds ratio greater than 2.0 with at least two confirmed cases, even if other samples did not pass molecular validation. The remaining 18 CNVs did not pass validation experiments.

One hundred thirty-nine of the 153 CNVs identified in high-risk ASD families were observed in case and/or control samples in this large dataset. Of these, 33 were present in two or more cases and had odds ratios greater than 2 and thus were selected for molecular confirmation. Following TaqMan validation, fifteen of the thirty-three CNVs were validated (Table 3). Of the 15 validated CNVs identified in high-risk families, 4 were shown to be inherited CNVs while three were de novo CNVs in the discovery families. The remainder were of undetermined origin, in most cases due to lack of information for one or both parents. A CNV that was validated in some samples but not in others, for example if a CNV was validated in all calls made by both PennCNV and CNAM but was not validated in all calls made only by one program, was considered to have passed validation if the validated samples yielded an odds ratio greater than 2.0 with at least two cases confirmed by validation.

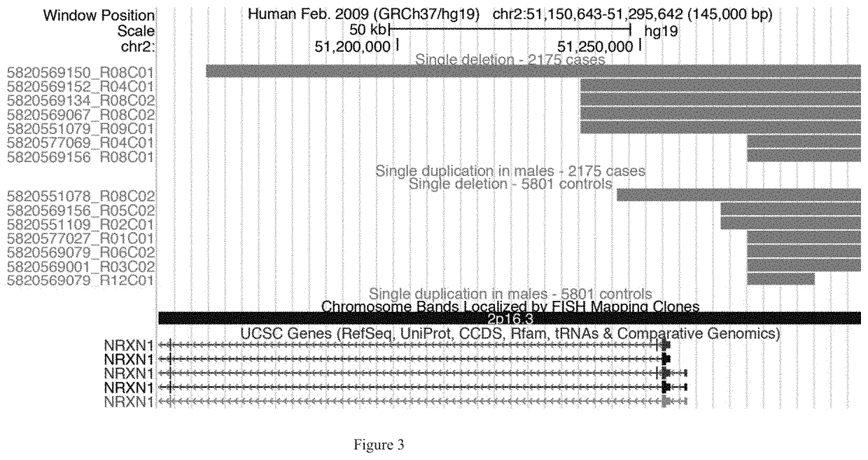

Notable among these CNVs is a deletion observed near the 5′-end of the NRXN1 gene. This deletion, observed in five cases and only in one control, includes at least a portion of the NRXNI-alpha promoter, and extends into the first exon of NLRXN1-α, as shown in the UCSC Genome Browser view [35] (FIG. 3). CNVs impacting NRXN1 in ASD as well as other neurological conditions have been published by others [15,32, 36-40], so the observation of NRXN1 CNVs both in our high-risk ASD family discovery work and in the large case/control replication study demonstrates our ability to detect biologically relevant CNVs that may also have clinical utility.

Other CNVs of interest included portions of the LINGO2 and STXBP5 genes. Single nucleotide variants in the LINGO2 gene have been associated with essential tremor and with Parkinson's disease, suggesting that the LINGO2 protein may have a neurological function [41]. However, CNVs in this gene have not previously been identified in individuals with ASD. We also observed deletions involving a portion of the STXBP5 gene, an interesting finding based on the potential role of STXBP5 in neurotransmitter release [42,43].

CNVs Identified by SNV Probes. Twenty-five additional CNVs shown in Table 3 were discovered using SNVs identified in our high-risk ASD families. The SNVs that detected these twenty-five CNVs (Table 10) were identified by exon capture and DNA sequencing in regions of haplotype sharing and in published ASD candidate genes in our high-risk ASD families, and were selected for further study because they might alter the function of the proteins in which they were found (unpublished observations). The 9 validated CNVs derived from SNV intensity data are shown in Table 3 (CNVs not detected in discovery cohort). One of these CNVs, a chromosome 15q duplication, encompasses three duplication CNVs in Table 10. These three CNVs are thought to be contiguous since TaqMan data confirmed the same samples to be positive for each of them.

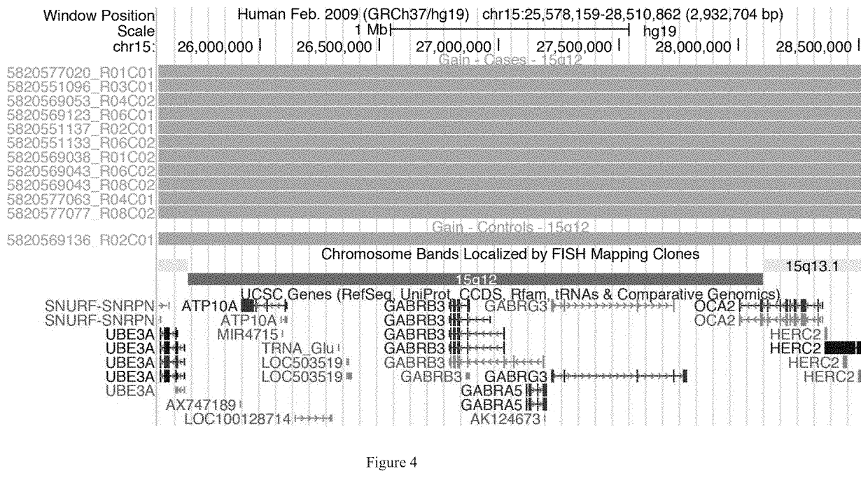

Interestingly, duplications involving the GABA receptor gene cluster, as well as many other genes, on chromosome 15q12 were observed in 11 unrelated cases in our study and only in a single control, shown in the UCSC Genome Browser view [35](Figure 4). Contrary to our findings, a recent search for CNVs in GABA pathway genes [44] did not find an enrichment of duplications in this region. Rather, both deletions and duplications were observed at similar frequencies in cases and controls.

Published CNVs. Additional CNVs from the literature and both published and unpublished CNVs identified at CHOP also were observed in our large dataset and met our criteria for potential clinical utility. Of those, 31 high-impact CNVs are shown in Table 4 (CNVs 20 and 21 in Table 4 are shown separately but are noted as likely being contiguous and thus likely are only a single entity). All CNVs not previously experimentally validated were validated in this study.

One of the previously unpublished CHOP CNVs is a duplication that encompasses the 3′-end RGS20 gene as well as the 3′-end of the TCEA1 gene. The RGS gene family encodes proteins that regulate G-protein signaling. These proteins function by increasing the inherent GTPase activity of their target G-proteins, and thus limit the signaling activity of their target G-proteins by keeping them in the inactive, GDP-bound state. RGS20 is expressed throughout the brain (reviewed in [45]), making it a likely candidate for involvement in neurological development. The TCEA1 gene, which also is partially encompassed by this CNV, is a transcription elongation factor involved in RNA polymerase II transcription. A role for TCEA1 in cell growth regulation has been suggested [46]. This potential role is consistent with the involvement of TCEA1 CNVs in ASD etiology as well.

TABLE 3
Validated CNVs discovered using affected children from Utah families.

| | | | CNV Region- | CNV Region- | CNV | Odds | | | | |
|---|---|---|---|---|---|---|---|---|---|---|
| No. | CNV Origin | Cytoband | Discovery Cohort | Replication Cohort | Type | Ratio | P Value | Cases | Controls | Gene/Region |
| 1 | Utah CNV | 1q21.1 | chr1: 145714421- | chr1: 145703115- | Dup | 3.37 | 9.60E−03 | 9 | 10 | CD160, PDZK1 |
| | | | 146101228 | 145736438 | | | | | | |
| 2 | Utah CNV | 1q41 | chr1: 215858193- | chr1: 215854466- | Del | 2.12 | 5.02E−03 | 22 | 39 | USH2A |
| | | | 215861879 | 215861792 | | | | | | |
| 3 | Utah CNV | 2p16.3 | chr2: 51272055- | chr2: 51266798- | Del | 14.96 | 8.26E−03 | 4 | 1 | upstream of NRXN1 |
| | | | 51336043 | 51339236 | | | | | | |
| 4 | Utah CNV^{#} | 3q26.31 | chr3: 172596081- | chr3: 172591359- | Dup | 3.74 | 2.11E−01 | 1 | 1 | downstream of SPATA16 |
| | | | 172617355 | 172604675 | | | | | | |
| 5 | Utah CNV^{#} | 4q35.2 | chr4: 189084983- | chr4: 189084240- | Del | 3.74 | 1.98E−01 | 2 | 2 | downstream of TRIML1 |
| | | | 189117429 | 189117031 | | | | | | |
| 6 | Utah CNV^{#} | 6p24.3 | chr6: 7425246- | chr6: 7461346- | Del | ∞ | 2.11E−01 | 1 | 0 | between RIOK1 and DSP |
| | | | 7464367 | 7470321 | | | | | | |
| 7 | Utah CNV^{#} | 6q11.1 | chr6: 62443739- | chr6: 62426827- | Dup | 3.74 | 1.98E−01 | 2 | 2 | KHDRBS2 |
| | | | 62462295 | 62472074 | | | | | | |
| 8 | Utah CNV | 6q24.3 | chr6: 147588752- | chr6: 147577803- | Del | ∞ | 2.10E−01 | 1 | 0 | STXBP5 |
| | | | 147664671 | 147684318 | | | | | | |
| 9 | Utah CNV^{#} | 7p22.1 | chr7: 6838712- | chr7: 6870635- | Dup | 7.47 | 1.15E−01 | 2 | 1 | upstream of CCZ1B |
| | | | 6864071 | 6871412 | | | | | | |
| 10 | Sequence | 7q21.3 | Not found | chr7: 93070811- | Del | ∞ | 4.46E−02 | 2 | 0 | CALCR, MIR653, MIR489 |
| | SNP CNV^{#} | | | 93116320 | | | | | | |
| 11 | Utah CNV^{#} | 9p21.1 | chr9: 28190069- | chr9: 28207468- | Del | 3.74 | 6.72E−02 | 4 | 4 | LINGO2 |
| | | | 28347679 | 28348133 | | | | | | |
| 12 | Utah CNV^{#} | 9p21.1 | chr9: 28190069- | chr9: 28354180- | Del | 3.73 | 3.78E−01 | 1 | 1 | LINGO2 (intron) |
| | | | 28347679 | 28354967 | | | | | | |
| 13 | Utah CNV | 10q23.1 | chr10: 83893626- | chr10: 83886963- | Del | 3.76 | 1.54E−02 | 7 | 7 | NRG3 (intron) |
| | | | 84175018 | 83888343 | | | | | | |
| 14 | Utah CNV^{#} | 10q23.31 | chr10: 92274764- | chr10: 92262627- | Dup | 7.47 | 1.15E−01 | 2 | 1 | downstream of |
| | | | 92289762 | 92298079 | | | | | | BC037970 |
| 15 | Utah CNV^{#} | 12q23.2 | chr12: 102097012- | chr12: 102095178- | Dup | 7.47 | 1.15E−01 | 2 | 1 | CHPT1 |
| | | | 102106306 | 102108946 | | | | | | |
| 16 | Utah CNV^{#} | 13q13.3 | chr13: 40087689- | chr13: 40089105- | Del | ∞ | 2.11E−01 | 1 | 0 | LHFP (intron) |
| | | | 40088007 | 40090197 | | | | | | |
| 17 | Sequence | 14q32.2 | Not found | chr14: 100705631- | Dup | 9.36 | 5.99E−03 | 5 | 2 | SLC25A29, YY1, MIR345, |
| | SNP CNV^{#} | | | 100828134 | | | | | | SLC25A47, WARS |
| 18 | Sequence | 14q32.31 | Not found | chr14: 102018946- | Dup | 4.62 | 1.01E−14 | 60 | 50 | DIO3AS, DIO3OS |
| | SNP CNV^{#} | | | 102026138 | | | | | | |
| 19 | Sequence | 14q32.31 | Not found | chr14: 102729881- | Del | 7.47 | 1.15E−01 | 2 | 1 | MOK |
| | SNP CNV^{#} | | | 102749930 | | | | | | |
| 20 | Sequence | 14q32.31 | Not found | chr14: 102973910- | Dup | 3.82 | 8.29E−26 | 136 | 142 | ANKRD9 (RAGE) |
| | SNP CNV^{#} | | | 102975572 | | | | | | |
| 21 | Sequence | 15q11.2- | Not found | chr15: 25690465- | Dup* | 41.05 | 1.82E−08 | 11 | 1 | ATP10A, GABRB3, |
| | SNP CNV^{#} | q13.1 | | 28513763 | | | | | | GABRA5, GABRG3, |
| 22 | Sequence | 15q13.2- | Not found | chr15: 31092983- | Del | ∞ | 4.46E−02 | 2 | 0 | FAN1, MTMR10, MIR211, |
| | SNP CNV^{#} | 15q13.3 | | 31369123 | | | | | | TRPM1 |
| 23 | Sequence | 15q13.3 | Not found | chr15: 31776648- | Dup | 4.40 | 6.91E−06 | 21 | 18 | OTUD7A |
| | SNP CNV^{#} | | | 31822910 | | | | | | |
| 24 | Sequence | 20q11.22 | Not found | chr20: 32210931- | Dup | 2.72 | 3.16E−02 | 8 | 11 | NECAB3, CBFA2T2, |
| | SNP CNV^{#} | | | 32441302 | | | | | | C20orf144, NECAB3, |

CNVs shown here were selected based on their p value, their case/control odds ratio, or both and were subject to molecular validation.
*This CNV is contiguous with the chromosome 15q11.2 CNV described in Table 4 based on TagMan data.
^{#}Designates CNVs not previously seen in ASD, based on queries for genes included in or flanking the CNV.
**Denotes gene in or adjacent to the CNV that is involved in neural function, development and disease (see Table 5-6).

TABLE 4
Published CNVs observed in our sample population.

| | | | Region of Highest | CNV | TaqMan | | | | | |
|---|---|---|---|---|---|---|---|---|---|---|
| No. | Cytoband | Literature CNVs | Significance | Type | Validation | OddsRatio | P Value | Cases | Ctrls | Gene/Region |
| 1 | 1q21.1 | chr1: 146555186- | chr1: 146656292- | Dup | NT | 7.48 | 1.15E−01 | 2 | 1 | FMO5 |
| | | 147779086 | 146707824 | | | | | | | |
| 2 | 2p24.3 | chr2: 13202218- | chr2: 13203874- | Del | Validated | ∞ | 2.11E−01 | 1 | 0 | upstream of |
| | | 13248445 | 13209245 | | (chr2: 13203874- | | | | | LOC100506474 |
| | | | | | 13209245) | | | | | |
| 3 | 2p21 | chr2: 45455651- | chr2: 45489954- | Dup | NT | ∞ | 4.46E−02 | 2 | 0 | between UNQ6975 |
| | | 45984915 | 45492582 | | | | | | | and SRBD1 |
| 4 | 2p16.3 | chr2: 50145644- | chr2: 51237767- | Del | NT | ∞ | 1.99E−03 | 4 | 0 | NRXN1** |
| | | 51259671 | 51245359 | | | | | | | |
| 5 | 2p15 | chr2: 62258231- | chr2: 62230970- | Dup | NT | ∞ | 2.11E−01 | 1 | 0 | COMMD1 |
| | | 63028717 | 62367720 | | | | | | | |
| 6 | 2q14.1 | chr2: 115139568- | chr2: 115133493- | Del | NT | 7.47 | 1.15E−01 | 2 | 1 | between |
| | | 115617934 | 115140263 | | | | | | | LOC440900 and |
| | | | | | | | | | | DPP10** |
| 7 | 3p26.3 | chr3: 1940192- | chr3: 1937796- | Del | Validated | 5.60 | 6.70E−02 | 3 | 2 | between CNTN6 |
| | | 1940920 | 1941004 | | (chr3: 1937796- | | | | | and CNTN4** |
| | | | | | 1942764) | | | | | |
| 8 | 3p14.1 | chr3: 67656832- | chr3: 67657429- | Del | NT | ∞ | 2.11E−01 | 1 | 0 | SUCLG2, FAM19A4, |
| | | 68957204 | 68962928 | | | | | | | FAM19A1 |
| 9 | 4q13.3 | chr4: 73756500- | chr4: 73766964- | Dup | Validated | ∞ | 2.11E−01 | 1 | 0 | COX18, ANKRD17 |
| | | 73905356 | 73816870 | | (chr4: 73753294- | | | | | |
| | | | | | 74058988) | | | | | |
| 10 | 4q33 | chr4: 154087652- | chr4: 171366005- | Del | NT | ∞ | 4.46E−02 | 2 | 0 | between AADAT** |
| | | 172339893 | 171471530 | | | | | | | and HSP90AA6P |
| 11 | 5q23.1 | chr5: 118478541- | chr5: 118527524- | Dup | Validated | 3.74 | 1.98E−01 | 2 | 2 | DMXL1, TNFAIP8 |
| | | 118584821 | 118589485 | | (chr5: 118527524- | | | | | |
| | | | | | 118614781) | | | | | |
| 12 | 6p21.2 | chr6: 39071841- | chr6: 39069291- | Del | Validated | 2.37 | 1.93E−02 | 12 | 19 | SAYSD1 |
| | | 39082863 | 39072241 | | (chr6: 39069291- | | | | | |
| | | | | | 39072241) | | | | | |
| 13 | 8q11.23 | chr8: 54858496- | chr8: 54855680- | Dup | Validated | ∞ | 2.11E−01 | 1 | 0 | RGS20, TCEA1 |
| | | 54907579 | 54912001 | | (chr8: 54855680- | | | | | |
| | | | | | 54912001) | | | | | |
| 14 | 10q11.22 | chr10: 46269076- | chr10: 49370090- | Dup | NT | 3.77 | 1.96E−01 | 2 | 2 | FRMPD2P1, |
| | | 50892143 | 49471091 | | | | | | | FRMPD2 |
| 15 | 10q11.23 | chr10: 50892146- | chr10: 50884949- | Dup | NT | 3.74 | 1.98E−01 | 2 | 2 | OGDHL, C10orf53 |
| | | 51450787 | 50943185 | | | | | | | |
| 16 | 12q13.13 | chr12: 53183470- | chr12: 53177144- | Del | Validated | ∞ | 4.46E−02 | 2 | 0 | between KRT76 and |
| | | 53189890 | 53180552 | | (chr12: 53177144- | | | | | KRT3 |
| | | | | | 53182177) | | | | | |
| 17 | 15q11.1 | chr15: 20266959- | chr15: 20192970- | Dup | Validated | 4.97 | 4.06E−02 | 4 | 3 | downstream of |
| | | 25480660 | 20197164 | | (chr15: 20192970- | | | | | HERC2P3 |
| | | | | | 20212798) | | | | | |
| 18 | 15q11.2 | chr15: 20266959- | chr15: 25099351- | Del | NT | 3.75 | 1.13E−01 | 3 | 3 | SNRPN** |
| | | 25480660 | 25102073 | | | | | | | |
| 19 | 15q11.2 | chr15: 20266959- | chr15: 25099351- | Dup | NT | 45.19 | 7.93E−08 | 12 | 1 | SNRPN** |
| | | 25480660 | 25102073 | | | | | | | |
| 20 | 15q11.2 | chr15: 25582397- | chr15: 25579767- | Dup* | Validated | ∞ | 3.86E−06 | 8 | 0 | between |
| | | 25684125 | 25581658 | | (chr15: 25576642- | | | | | SNORD109A and |
| | | | | | 25581880) | | | | | UBE3A** |
| 21 | 15q11.2 | chr15: 25582397- | chr15: 25582882- | Dup* | NT | 30.08 | 2.82E−05 | 8 | 1 | UBE3A** |
| | | 25684125 | 25662988 | | | | | | | |
| 22 | 16p12.2 | chr16: 21901310- | chr16: 21958486- | Dup | NT | ∞ | 4.47E−02 | 2 | 0 | C16orf52, |
| | | 22703860 | 22172866 | | | | | | | UQCRC2**, PDZD9, |
| | | | | | | | | | | VWA3A |
| 23 | 16p11.2 | chr16: 29671216- | chr16: 29664753- | Del | NT | 7.47 | 1.15E−01 | 2 | 1 | DOC2A**, ASPHD1, |
| | | 30173786 | 30177298 | | | | | | | LOC440356, TBX6, |
| | | | | | | | | | | LOC100271831, |
| | | | | | | | | | | PRRT2 |
| | | | | | | | | | | CDIPT, QPRT, YPEL3, |
| | | | | | | | | | | PPP4C, MAPK3**, |
| | | | | | | | | | | SPN, MVP, FAM57B, |
| | | | | | | | | | | ZG16, ALDOA, |
| | | | | | | | | | | INO80E, SEZ6L2, |
| | | | | | | | | | | TAOK2, KCTD13, |
| | | | | | | | | | | MAZ, KIF22, GDPD3, |
| | | | | | | | | | | C16orf92, C16orf53, |
| | | | | | | | | | | TMEM219, |
| | | | | | | | | | | C16orf54, HIRIP3 |
| 24 | 16q23.3 | chr16: 82195236- | chr16: 82423855- | Dup | NT | ∞ | 4.46E−02 | 2 | 0 | between |
| | | 82722082 | 82445055 | | | | | | | MPHOSPH6 and |
| | | | | | | | | | | CDH13 |
| 25 | 17p12 | chr17: 14139846- | chr17: 14132271- | Dup | Validated | 1.60 | 3.57E−01 | 3 | 7 | between COX10 and |
| | | 15282723 | 14133349 | | (chr17: 14132271- | | | | | CDRT15 |
| | | | | | 14133568) | | | | | |
| 26 | 17p12 | chr17: 14139846- | chr17: 14132271- | Del | NT | 5.61 | 6.70E−02 | 3 | 2 | PMP22**, CDRT15, |
| | | 15282723 | 15282708 | | | | | | | TEKT3, MGC12916, |
| | | | | | | | | | | CDRT7, HS3ST3B1 |
| 27 | 17p12 | chr17: 14139846- | chr17: 14952999- | Dup | NT | 3.74 | 1.98E−01 | 2 | 2 | between CDRT7 and |
| | | 15282723 | 15053648 | | | | | | | PMP22 |
| 28 | 17p12 | chr17: 14139846- | chr17: 15283960- | Del | Validated | 3.74 | 1.13E−01 | 3 | 3 | between TEKT3 and |
| | | 15282723 | 15287134 | | (chr17: 15283960- | | | | | FAM18B2-CDRT4 |
| | | | | | 15287134) | | | | | |
| 29 | 20p12.3 | chr20: 8044044- | chr20: 8162278- | Dup | NT | 3.73 | 1.98E−01 | 2 | 2 | PLCB1** |
| | | 8527513 | 8313229 | | | | | | | |
| 30 | Xp21.2 | chrX: 28605682- | chrX: 29944502- | Dup | NT | ∞ | 4.47E−02 | 2 | 0 | IL1RAPL1** |
| | | 29974014 | 29987870 | | | | | | | |
| 31 | Xq27.2 | chrX: 139998330- | chrX: 140329633- | Del | Validated | 7.48 | 2.06E−02 | 4 | 2 | SPANXC |
| | | 140443613 | 140348506 | | (chrX: 140329633- | | | | | |
| | | | | | 140456325) | | | | | |
| 32 | Xq28 | chrX: 148858522- | chrX: 148882559- | Del | Validated | ∞ | 4.46E−02 | 2 | 0 | MAGEA8 |
| | | 149097275 | 148886166 | | (chrX: 148882559- | | | | | |
| | | | | | 149020410) | | | | | |

*Denotes CNVs contiguous with the chromosome 15g11.2-13.1 CNVs shown in Table 3.
**Denotes gene in or adjacent to the CNV that is involved in neural function, development and disease (see Table 5-6).

Pathway analysis. Analysis of 104 genes within or immediately flanking our PCR-validated CNVs yielded significant association of these genes to previously characterized functional networks. The five most statistically significant networks, along with their statistical scores, are shown in Table 5. The top ranking functional categories identified in this analysis, along with their P-values, are shown in Table 6.

TABLE 5
Top Significant Networks Identified by
Pathway Analysis using Ingenuity IPA.

| Network | Score |
|---|---|
| Cell-To-Cell Signaling and Interaction, Tissue | 55 |
| Development, Gene Expression | |
| Neurological Disease, Behavior, Cardiovascular Disease | 28 |
| Cell Death, Cellular Compromise, Neurological Disease | 26 |
| Cellular Development, Cell Morphology, Nervous System | 20 |
| Development and Function | |
| Behavior, Cardiovascular Disease, Neurological Disease | 18 |

Network scores are the −log P for the results of a right-tailed Fisher's Exact Test.

As expected for CNVs associated with a neurodevelopmental disorder, a significant number of genes in or adjacent to the CNVs described here are involved in neural function, development and disease (Tables 5-6). Examples of such genes include: GABRA5, GABRA3, GABRG3, UBE3A, E2F1, PLCB1, PMP22, AADAT, MAPK3, NRXN1, NRG3, DPP10, UQCRC2, USH2A, NECAB3, CNTN4, LINGO2, IL1RAPL1, STXBP5, DOC2A, and SNRPN. Of these genes, E2F1, AADAT, NECAB3, and IL1RAPL1 are not found in the Autism Chromosome Rearrangement Database (see website at projects.tcag.ca/autism/), suggesting that they may be novel ASD risk genes.

The novel ASD risk loci identified here have functions that suggest a significant role in brain function and architecture. As such, altering the function of each of these genes as a result of the CNV could impinge on the biochemical pathways that are relevant to ASD etiology.

For example, mutations in IL1RAPL1 have been observed in cases of X-linked intellectual disability [47], and the encoded protein has been shown to play a role in voltage-gated calcium channel regulation in cultured cells [48]. E2F1 encodes a transcription factor and DNA-binding protein that plays a significant role in regulating cell growth and differentiation, apoptosis and response to DNA damage (reviewed in Biswas and Johnson, 2012 [49]). Each of these genes thus could have detrimental impacts on normal brain function.

NECAB3 encodes a neuronal protein with two isoforms that regulate the production of beta-amyloid peptide in opposite directions, depending on whether exon 9 of NECAB3 is included in or excluded from the mature mRNA [50].

AADAT encodes an aminotransferase with multiple functions, one of which leads to the synthesis of kynurenic acid. This pathway has been proposed as a target for potential neuroprotective therapeutics, indicating the potential significance of this finding for ASD etiology (reviewed in Stone et al., 2012 [51]). The specific roles that any of these genes play in ASD etiology have yet to be determined, but the observed neurological functions of their encoded proteins strongly support a potential role in normal brain function.

Many of these genes also have been implicated in other nervous system disorders, including Huntington's, Parkinson's, and Alzheimer's diseases as well as schizophrenia and epilepsy [41, 52-61]. One of the features common to this group of disorders, which includes ASD, is synaptic dysfunction. There is a significant overlap in genes, and/or the molecular mechanisms by which these genes give rise to synaptopathies (reviewed in [62]). We therefore find it notable that many such genes involved in other synaptopathies were found within or flanking the validated CNVs we identified as associated with ASD.

In addition to neurogenic genes, validated CNVs were associated with genes with known roles in renal and cardiovascular diseases (Table 6). Several syndromic forms of autism, such as DiGeorge Syndrome and Charcot-Marie Tooth Disease are comorbid with renal and cardiovascular disease, and therefore it was not surprising to find that our study identified CNVs containing genes associated with these syndromes and functions, such as CDRT15, and CDH13.

TABLE 6
Top Significant Biological Functions Identified
by Ingenuity IPA and Literature Searches.

| Function | p-value range | # Genes |
|---|---|---|
| Neurological Disease | 2.71E−05-3.15E−02 | 14 (18) |
| Behavior | 5.93E−05-4.36E−02 | 10 |
| Cardiovascular Disease | 8.58E−05-4.30E−02 | 10 |
| Cellular Development | 1.39E−04-4.77E−02 | 9 |
| Inflammatory response | 4.84E−04-2.89E−02 | 6 |

The right-tailed Fisher's exact test was used to calculate P-values representing the probability that selecting genes associated with that pathway or network is due to chance alone. Each functional category represents a collection of associated subcategories, each of which has an associated P-value. For example, within ‘Neurological Disease,’ are subcategories of genes associated with seizures, Huntington Disease, schizophrenia, etc. The P-value range range given represents the range of P-values generated for each subcategory. In the first line, 36 genes were associated with a function in Neurological Disease by Ingenuity software. An additional 11 genes were identified as having neurological functions in the literature, giving a total of 47 with known or suspected roles in neurological disease.

There is mounting evidence, as well, that inflammatory responses are involved with the development and progression of autism (reviewed in [63]). Maternal immune activation during pregnancy is believed to activate fetal inflammatory responses, in some cases with detrimental effects on neural development in the fetus, leading to autism. This environmental insult could be mediated or enhanced by genomic changes that predispose the fetus to elevated inflammatory responses, so it is significant that a number of genes from our validated CNVs play a role in inflammatory response. Examples of these include CD160, CALCR, and SPN.

Our findings are consistent with other studies that used pathway analysis to characterize the genes contained in ASD risk CNVs, and suggest that many different biological pathways, when disrupted, can lead to features observed in ASD. The wide variety of biological functions identified for these genes also is consistent with estimates of the number of independent genetic variants that may play a role in the etiology of ASD (8-11).

### Discussion

We used a custom microarray to characterize the frequency of CNVs identified in high-risk ASD families in a large ASD case/control population. We also evaluated further the frequency of CNVs discovered in several published studies in our sample cohort to obtain a clearer picture of the potential clinical utility of these CNVs in the genetic evaluation of children with ASD. We used multiple quality control measures to insure that all cases and controls a) had no unexpected familial relationships; b) represented a uniform ethnic group; c) were devoid of uncharacterized whole chromosome anomalies or other genomic abnormalities consistent with syndromic forms of ASD; d) had sufficient power to distinguish risk variants from CNVs with little or no impact on the ASD phenotype; and e) were validated using quantitative PCR even though the custom array used here represented at least a second evaluation for most of them. Parents of ASD cases tested were not available to determine state of inheritance.

The validity of this approach was confirmed by our observation of CNVs that had been previously identified as ASD risked markers, including CNVs encompassing parts of the NRXN1 gene. CNVs and point mutations in NRXN1 are thought to play a role in a subset of ASD cases as well as in other neuropsychiatric conditions [15,32,36-40]. The data from our study demonstrate that NRXN1 CNVs also occur in high-risk ASD families. Further, our case/control data provide additional evidence that neurexin-1 plays an important role in unrelated ASD cases. While CNVs near NRXN1 occur in controls as well as in cases, the CVNs observed in our ASD cases typically disrupt a portion of the NRXN1 coding region while CNVs observed in our control population do not.

CNVs from high-risk ASD families. In the high-risk ASD families, both novel and previously observed CNVs were identified that contain genes with potential relevance to neuropsychiatric conditions such as ASD. These include CNVs involving LINGO2, the GABR gene cluster on chromosome 15q12 and STXBP5. Each of these CNV regions has an odds ratio greater than 2 and most of the CNVs we identified in high-risk families have a significant p value associating them with the ASD phenotype in this case/control study. Some CNVs, although observed only in ASD cases and not in controls, were too rare even in this large dataset to generate statistically significant results. An example is a deletion involving STXBP5 that was observed two ASD samples and in no controls. A deletion including this gene was previously observed in a patient with an apparent syndromic form of ASD [64], lending further support to our observation of STXBP5 deletions in ASD cases. These data collectively suggest that CNVs observed in high-risk ASD families also are important contributors to the etiology of ASD in an ASD case/control population.

We detected rare duplications involving the GABA receptor gene cluster as well as additional genes in the Prader-Willi/Angelman syndrome region on chromosome 15 (11/1,544 unrelated cases, 1/5,762 unrelated controls, OR=40.05). All of these CNVs were confirmed using TaqMan assays spanning the region, and these results strongly suggest a role for duplications on chromosome 15q12 in ASD etiology. Deficiency of GABA_{A} receptors indeed is thought to play an important role in both autism and epilepsy, and duplications have been observed to result in decreased GABR expression through a potential epigenetic mechanism (reviewed in [65]). Further, differences in the expression of GABRB3 mRNA and protein in the brains of some children with autism have been reported along with loss of biallelic expression of the chromosome 15q GABR genes in some individuals, [66], suggesting that epigenetic regulation of the chromosome 15 GABR gene cluster could also contribute to ASD etiology. Consistent with many previous findings from family studies, case reports and modest case/control studies (see website at omim.org/entry/608636), our data provide additional support for the involvement of duplications in this region of the genome in ASD. Further, our large population study suggests that these duplications may explain as much as 0.7% of ASD cases.

A recent study searching for CNVs encompassing genes in the GABA pathway, including the chromosome 15 GABR gene cluster, also found CNVs in this region. In contrast to our findings, this study found GABR gene cluster duplications at similar frequencies in both cases and in controls (Table S2 in ref. [44]). In addition, deletions were more common in this study in both cases and controls, while duplications were more common in our data. The differences between the two studies may lie in the sample population being studied, the uniformity of our sample population, or the technology platform used for CNV discovery (custom Illumina array compared to a custom Agilent array). Previous results have demonstrated maternal inheritance of deletions in this region in children with autism [67]. However, in our family studies we did not observe CNVs involving chromosome 15q12, and our case/control data preclude us from determining the parent of origin.

Interestingly, the CNVs that we observed on chromosome 15q were detected primarily with probes for SNVs identified in the GABR genes. Further, these SNVs were identified in affected individuals from high-risk ASD families. We did not observe CNVs involving this region in our high-risk ASD families. The observation of frequent duplications in our case/control population in the region containing these genes, coupled with the detection of these CNVs using probes for potential detrimental single nucleotide variants, suggests that both SNVs and CNVs involving the GABR genes might be pathogenic.

Literature supported CNVs. In addition to the CNVs identified in our high-risk ASD families, we evaluated further ASD risk CNVs identified in previous studies. Our results (Table 4) clearly demonstrate a role for many of these CNVs in ASD pathogenesis. Consistent with previous results, our data demonstrate in a large ASD population that rare CNVs are likely to play a role in the genetics of ASD, and suggest that these CNVs should be included in the genetic evaluation of children with ASD.

Interestingly, recent publications have identified a recurrent duplication of the Williams syndrome region on chromosome 7q11.23 in children with ASD [9,11]. We included probes for this region on our custom array, and were not able to identify any 7q11.23 duplications in our datasets. The reason(s) we did not observe any duplications in this region is not obvious; we had adequate probe coverage to have seen such duplications if they were present. Similar to the simplex ASD families used in those published studies, most of our ASD samples also were from reported simplex families, so the lack of observation of these CNVs is unlikely to be due to differences in family structure.

A CNV discovered at CHOP and not previously published includes a portion of the LCE gene cluster on chromosome 1. Deletions in this region have been associated with psoriasis [68,69], but no variants in this region have been I inked to autism. Focusing solely on individuals of Caucasian ancestry, we observed this CNV deletion in a single case and also a single control. However, when we included samples of non-Caucasian or uncertain ancestry, we observed 27 additional case DNA samples that carried this deletion, while only a single additional CNV-positive control was observed. Interestingly, based on SNP genotype results from principal component analysis, all of the cases that were positive for this CNV were of Asian descent. Since our control cohort had few individuals of Asian descent, we suspected that this CNV might be common in the Asian population. Analysis of whole genome data for individuals of non-Caucasian ancestry genotyped at the Center for Applied Genomics did not demonstrate common CNVs in either cases or controls in this region in individuals with Asian ancestry. However, a common CNV including LCE3E was observed in individuals with African ancestry (unpublished observations). Further analysis will be necessary to determine if this CNV is an ASD risk variant in either Asian or African populations.

Effect of analysis method on CNV validation. Although some CNVs are described here for the first time, many of the CNVs that we evaluated in this study were described previously. It is interesting to note that individual CNV calls that were made with both of the software packages we used were much more likely to be validated by qPCR than were CNVs called by either program alone. In fact, 97% of the CNVs called by both PennCNV and CNAM validated using TaqMan qPCR assays, while only 24% of the CNVs called by PennCNV alone and 30% of the CNVs called by CNAM alone were validated using the same approach. The concordance between the two analysis methods is informative given that the final sample sets used by the two methods differed substantially. The CNAM analysis used 290 fewer case samples and 575 fewer control samples than the PennCNV analysis. These data clearly demonstrate the value of using multiple software packages to evaluate microarray data for CNV discovery work. Our data are consistent with the rarity of many CNVs detected in DNA from children with ASD, and with the suggestion that there may be hundreds of loci that contribute to the development of ASD [9,11].

Our data demonstrate that CNVs identified in high-risk ASD families play a role in the etiology of ASD in unrelated cases. Evaluation of these CNVs in the large sample set used in this study provides compelling evidence for extremely rare recurrent CNVs as well as additional common variants in the genetics of ASD. We suggest that the CNVs described here likely have a strong impact on the development of ASD. Given the extensive quality control measures we used to characterize our sample cohort, the frequency at which we observed these CNVs in our cohort, and the molecular validation that we used to verify the calls, these CNVs can be used to increase sensitivity in the genetic evaluation of children with ASD. Further work will help to determine if the CNVs reported here are important for specific clinical subsets of ASD cases.

### Supplemental Methods

Samples: All high risk ASD family members and controls were of self-reported European ancestry. Among all cases in the replication study, 84% were of self-reported European ancestry, 6% were of self-reported African ancestry, 5% were self-reported as having multiple ethnic origins, and 5% were of unknown ethnicity. Among the cases, 1,577 were reported from unique families, 864 from 432 different families with 2 siblings, 369 from 123 different families with 3 siblings, 172 from 43 different families of 4 siblings, 5 siblings from a single family, 6 siblings from a single family, and 7 siblings from a single family. Among the DNA from cases used for genotyping, 1% came from cell pellets, 61% come from lymphoblastoid cell lines, 35% came from whole blood, and for 3% the source of DNA remained unknown. DNA was extracted from cell lines or lymphocytes, and quantitated using UV spectrophotometry. Six thousand controls were recruited by CHOP after obtaining informed consent under an IRB approved protocol. All DNA samples from controls were extracted from whole blood. Only individuals with self-reported Caucasian ancestry were used for this study. Pairwise identity by descent (IBD) was used to confirm known family assignments for cases, and to identify cryptic relatedness arising out of multiple subject enrollments across/within cohorts for all samples. Related individuals were removed so that only one family member remained in the study.

Array processing: We used 250 ng of genomic DNA to genotype each sample, according to the manufacturer's guidelines. On day one, genomic DNA was amplified 1000-1500-fold. Day two, amplified DNA was fragmented ˜300-600 bp, then precipitated and resuspended, followed by hybridization on to a BeadChip. Single base extension (SBE) utilizes a single probe sequence ˜50 bp long designed to hybridize immediately adjacent to the SNP query site. Following targeted hybridization to the bead array, the arrayed SNP locus-specific primers (attached to beads) were extended with a single hapten-labeled dideoxynucleotide in the SBE reaction. The haptens were subsequently detected by a multi-layer immunohistochemical sandwich assay, as recently described (Pastinen et al., 2000, Genome Res. 10, 1031, Erdogan et al., 2001, Nuc. Acids Res. 29, E36). The Illumina iScan was used to scan each BeadChip at two wavelengths and an image file was created. As BeadChip images were collected, intensity values were determined for all instances of each bead type, and data files were created that summarized intensity values for each bead type. These files were loaded directly into Illumina's genotype analysis software, BeadStudio. A bead pool manifest created from the LIMS database containing all the BeadChip data was loaded into BeadStudio along with the intensity data for the samples. BeadStudio used a normalization algorithm to minimize BeadChip to BeadChip variability. Once the normalization was complete, the clustering algorithm was run to evaluate cluster positions for each locus and assign individual genotypes. Each locus was given an overall score based on the quality of the clustering and each individual genotype call was given a GenCall score. GenCall scores provided a quality metric that ranges from 0 to 1 assigned to every genotype called. GenCall scores were then calculated using information from the clustering of the samples. The location of each genotype relative to its assigned cluster determined its GenCall score.

Sample quality control: Quality control measures were intended to identify the samples with the greatest probability of successful CNV identification and to remove the samples with features making CNV identification problematic. Most of the QC metrics employed were originally designed for applications involving high-density genome-wide data. For this study, it was deemed possible that an otherwise high-quality sample with a few large CNVs might fail some QC metrics due to the sparse nature of the data from the custom array employed. The QC process was therefore approached with caution, and inclusion criteria were determined by manual review of the data for each metric in order to identify the outlier values.

Derivative log ratio spread (DLRS): Derivative Log Ratio Spread (DLRS) is a measurement of point-to-point consistency of LR data, and is a reflection of the signal-to-noise ratio. It is similar in nature to the standard deviation of LR values that is often used in CNV studies, but has the advantage of being robust against large CNVs, which may influence standard deviation. DLRS was calculated for each chromosome, and the median chromosome DLRS value was used as a quality test. The distribution of the median DLRS statistic can be seen below. The outlier threshold was set at 0.3. One hundred twenty-eight subjects fail at this threshold, including all of the 75 samples that failed the waviness factor QC metric (see below).

Waviness factor. The “waviness” of each sample in the study was measured using the method of Diskin, et al. [27] as employed within SVS. An absolute value of 0.2 was determined as the outlier threshold for this metric, and 75 subjects failed at this threshold.

Chromosomal Abnormalities and Cell-Line Artifacts: Fifty-one samples (12 cases and 39 controls) were determined to have a chromosome 21 trisomy, consistent with a diagnosis of Down syndrome. These subjects were later confirmed to have Down syndrome based on clinical data review, and were removed from all further analyses. Additionally, 10 samples were removed based on other abnormalities that appeared to affect entire chromosomes.

Excessive CNVs: During the course of our analysis, several subjects were noted, using heat map style plots, to have a high frequency of copy number variant regions, in particular copy number gains. To identify the problematic subjects, we estimated the proportion of autosomal CNV regions in the data for which each subject had any CNV gain or loss. After manual review of the distribution of this proportion, 17 subjects with CNV calls at more than 10% of the regions were dropped from further analysis.

Principle component analysis (PCA). Substantial stratification was observed in the LR intensity data. The first two components were stratified by gender, and additional stratification and clustering was observed in the higher components as well. It was therefore considered prudent to apply a PCA correction to the intensity data prior to analysis in order to reduce the probability of data artifacts influencing CNV calls. The principal components were calculated based on all 9,000 samples in the QC process and the results were skewed by the presence of low quality samples. The principle components were therefore recalculated for the 8,777 samples passing preliminary QC, including samples that passed the tests for waviness, DLRS, PCA outliers, chromosome 21 trisomies, and the initial genotyping lab QC. After calculating the first 50 principal components and examining the distribution of eigenvalues, the LR values were corrected for 20 principal components, which were determined to be sufficient to explain the majority of variability in the data. The corrected LR data was then used for segmentation and CNV identification.

CNV calling: The segmentation covariates were reduced to a non-redundant spreadsheet, with columns for each marker position where at least one subject had an intensity shift. The distribution of values for each of these columns then was analyzed to determine if multiple copy number states were present, and if so, to estimate the threshold values that defined the different classes. The threshold values were first estimated by a simple algorithm that identified the mode of the distribution, and assuming this to be the neutral copy number state, set upper and lower thresholds based on the variance of the distribution. These thresholds were then manually reviewed, and gross errors were corrected as necessary. After threshold values were confirmed for each of the non-redundant regions, each subject's data for that region was classified accordingly as loss, gain, or neutral. These values were then used to populate a table of discrete copy number calls for use in association testing.

TaqMan assays: DNA samples and controls were transferred from stock tubes and diluted with molecular grade water to a final concentration of 5ng/ul into 0.75 mL Thermo Scientific Matrix storage tubes. All pipetting steps were carried out using Beckman Coulter Biomek FXp automation (Beckman Coulter, Inc., Fullerton, Calif., USA) unless otherwise stated. For each assay, 14 ul of each sample were plated into rows of a 96-well full-skirted plate. The last well in each row was left blank as a non-template control. Each quadrant of the 384-well reaction plates was stamped with 2 ul of DNA from the 96-well sample plate, so that each sample was assayed in quadruplicate. The reaction plates were dried and stored at 4° C. The TaqMan® reaction mix for each assay was prepared according to Applied Biosystems' (Applied Biosystems, Foster City, Calif., USA) recommendations with RNaseP as the reference assay (reference gene) and transferred by hand to each row of a 96-well full-skirted plate. 10 ul of each assay mix was then stamped into the appropriate reaction plate containing 10 ng of dried down DNA per well. The reaction plates were sealed with optical adhesive film, mixed on a plate vortex mixer, and centrifuged prior to running on the Applied Biosystems 7900HT Real Time PCR instrument. Thermal cycling was performed according to the manufacturer's recommended protocol (Applied Biosystems. Data were analyzed with SDS v2.4 software (Applied Biosystems). The baseline was calculated automatically and the threshold was set manually based on the exponential phase of the amplification plot. Data were exported as a text file and imported into the Applied Biosystems CopyCaller v2.0 Program. Assays were analyzed by setting a negative control sample (selected from samples showing none of the CNVs under study by either PennCNV or CNAM) copy number to n=2 except for X chromosome assays, which were analyzed using n=1. For X chromosome CNVs both male and female control samples were used (3 male, 2 female). All other parameters were left as default.

Pathway analysis. Ninety of the genes analyzed were within CNV duplications and 63 genes were within CNV deletions. Eighty-seven genes were included since they were the gene nearest to a validated intergenic CNV. Gene abbreviations were batch converted to their Entrez Gene IDs using G:CONVERT [31,32]. Both DAVID and Ingenuity IPA use the right-tailed Fisher's Exact test to calculate P-values representing the probability that selecting genes associated with that pathway or network is due to chance alone.

Network Generation using IPA: Each gene in our list of 240 was mapped to its corresponding object in Ingenuity's Knowledge Base. These genes were overlaid onto a global molecular network developed from information contained in Ingenuity's Knowledge Base. Networks then were algorithmically generated based on their connectivity. Both direct and indirect interactions were searched. Network scores are the −log P for the results of a right-tailed Fisher's Exact Test.

Principle component analysis (PCA) Results. Principal components analysis was used to assess the impact of population stratification within the study subjects. Principal components were calculated in SVS using default settings. All subjects were included in the calculation except those that failed data QC. Prior to calculating principal components, the SNPs were filtered so that only SNPs that met the following criteria were used: 1) autosomal SNPs only; 2) call rate >0.95; 3) MAF >0.05; 4) linkage disequilibrium R² <25% for all pairs of SNPs within a moving window of 50 SNPs. In total 2008 SNPs met these criteria. Self-reported ethnicity was used to group samples into “Caucasian” and “non-Caucasian” sets. A simple outlier detection algorithm was applied to stratify the subjects into the two groups. This was done by first calculating the Cartesian distance of each subject from the median centroid of the first two principal component vectors. After determining the third quartile (Q3) and inter-quartile range (IQR) of the distances, any subject with a distance exceeding Q3+1.5*IQR was determined to be outside of the main cluster, and therefore non-Caucasian. Five hundred sixty-four subjects were placed in the non-Caucasian category, including 207 cases and 57 controls. A small number of samples were removed due to duplicate enrollment in the study, but no other unexpected relationships were identified.

TABLE 7
TaqMan Assays Used for CNV Validation

| | Start Coord. | End Coord. | |
|---|---|---|---|
| Chromosome | (hg19) | (hg19) | Assay Name |
| chr1 | 145608130 | 145608131 | Hs01960835_cn |
| chr1 | 145714157 | 145714158 | Hs03356306 |
| chr1 | 145727743 | 145727744 | Hs02151880 |
| chr1 | 145831706 | 145831707 | Hs03363224_cn |
| chr1 | 215857628 | 215857629 | Hs06533545_cn |
| chr1 | 215860518 | 215860519 | Hs05788384_cn |
| chr2 | 13206303 | 13206304 | Hs05832292_cn |
| chr2 | 51257082 | 51257083 | Hs04675592_cn |
| chr2 | 51273782 | 51273783 | Hs03406712_cn |
| chr2 | 51335043 | 51335044 | Hs03207855_cn |
| chr2 | 78417269 | 78417270 | Hs03210777 |
| chr2 | 78448009 | 78448010 | Hs03219183 |
| chr3 | 1940242 | 1940243 | Hs03449476_cn |
| chr3 | 74559838 | 74559839 | Hs06657187_cn |
| chr3 | 74570239 | 74570240 | Hs03006662_cn |
| chr3 | 74580064 | 74580065 | Hs06656853_cn |
| chr3 | 172593661 | 172593662 | Hs05888850_cn |
| chr3 | 172600469 | 172600470 | Hs04760981_cn |
| chr3 | 174853869 | 174853870 | Hs03492315_cn |
| chr3 | 174889051 | 174889052 | Hs03463132_cn |
| chr3 | 176765106 | 176765107 | Hs00705847 |
| chr3 | 176773900 | 176773901 | Hs06653638 |
| chr3 | 178962631 | 178962632 | Hs04718548_cn |
| chr3 | 178969356 | 178969357 | Hs00989875_cn |
| chr4 | 73785471 | 73785472 | Hs04844255_cn |
| chr4 | 73923259 | 73923260 | Hs02916212_cn |
| chr4 | 74027025 | 74027026 | Hs00308217_cn |
| chr4 | 189089063 | 189089064 | Hs03238737 |
| chr4 | 189109145 | 189109146 | Hs03244159 |
| chr5 | 99647650 | 99647651 | Hs03245981_cn |
| chr5 | 99665469 | 99665470 | Hs03248003_cn |
| chr5 | 118544341 | 118544342 | Hs06046822_cn |
| chr5 | 118567989 | 118567990 | Hs03578408_cn |
| chr5 | 118606921 | 118606922 | Hs03562094_cn |
| chr6 | 7464166 | 7464167 | Hs03258806_cn |
| chr6 | 7467367 | 7467368 | Hs03261355_cn |
| chr6 | 39070306 | 39070307 | Hs06797005_cn |
| chr6 | 44131202 | 44131203 | Hs06765368_cn |
| chr6 | 49257472 | 49257473 | Hs06135362_cn |
| chr6 | 62432331 | 62432332 | Hs06740361_cn |
| chr6 | 62468865 | 62468866 | Hs06752297_cn |
| chr6 | 127449047 | 127449048 | Hs04898996 |
| chr6 | 127467261 | 127467262 | Hs06149095 |
| chr6 | 147599263 | 147599264 | Hs00462911_cn |
| chr6 | 147649513 | 147649514 | Hs06799063_cn |
| chr6 | 147681914 | 147681915 | Hs04903013_cn |
| chr7 | 6870706 | 6870707 | Hs03632408_cn |
| chr7 | 15383278 | 15383279 | CusTaq1CX6RM14_cn |
| chr7 | 15405201 | 15405202 | ContR26CX0IV8W_cn |
| chr7 | 93080844 | 93080845 | Hs04974410_cn |
| chr7 | 93145475 | 93145476 | Hs04971099_cn |
| chr7 | 93152478 | 93152479 | Hs04944233_cn |
| chr7 | 100232257 | 100232258 | Hs03629609 |
| chr7 | 100304948 | 100304949 | Hs01981045 |
| chr7 | 100381692 | 100381693 | Hs05013769 |
| chr7 | 124527535 | 124527536 | Hs03620793_cn |
| chr7 | 124578724 | 124578725 | Hs03650226_cn |
| chr7 | 149504056 | 149504057 | Hs03630536 |
| chr7 | 149528561 | 149528562 | Hs03645125 |
| chr7 | 149550437 | 149550438 | Hs03640597 |
| chr8 | 3165293 | 3165294 | Hs02622320_cn |
| chr8 | 54865516 | 54865517 | Hs03668894_cn |
| chr8 | 54905347 | 54905348 | Hs03694907_cn |
| chr8 | 84323860 | 84323861 | Hs04360657 |
| chr8 | 84331501 | 84331502 | Hs03658852 |
| chr8 | 85298919 | 85298920 | Hs03668441_cn |
| chr8 | 85303238 | 85303239 | Hs03678663_cn |
| chr8 | 86467253 | 86467254 | Hs03673176_cn |
| chr9 | 28203352 | 28203353 | Hs03707922_cn |
| chr9 | 28266812 | 28266813 | Hs03714527_cn |
| chr9 | 28333835 | 28333836 | Hs03725541_cn |
| chr9 | 28354528 | 28354529 | Hs03723870_cn |
| chr9 | 136523906 | 136523907 | Hs01617069_cn |
| chr9 | 136527743 | 136527744 | Hs06869845_cn |
| chr9 | 139091261 | 139091262 | Hs06889516_cn |
| chr9 | 139101729 | 139101730 | Hs06847090 |
| chr9 | 139110612 | 139110613 | Hs00495475 |
| chr10 | 83887149 | 83887150 | Hs03726621_cn |
| chr10 | 89717970 | 89717971 | Hs05212456 |
| chr10 | 92274027 | 92274028 | Hs03746257 |
| chr10 | 92287873 | 92287874 | Hs03740287 |
| chr12 | 53178157 | 53178158 | Hs06965067_cn |
| chr12 | 53181253 | 53181254 | Hs06930722_cn |
| chr12 | 71934616 | 71934617 | Hs06933395_cn |
| chr12 | 71950419 | 71950420 | Hs01107784_cn |
| chr12 | 73071721 | 73071722 | Hs06996317_cn |
| chr12 | 73094916 | 73094917 | Hs03093848_cn |
| chr12 | 80898972 | 80898973 | Hs03825941_cn |
| chr12 | 80974071 | 80974072 | Hs03820308_cn |
| chr12 | 81007496 | 81007497 | Hs03818167_cn |
| chr12 | 81610738 | 81610739 | Hs00229436_cn |
| chr12 | 81693094 | 81693095 | Hs00586334_cn |
| chr12 | 81746602 | 81746603 | Hs06985491_cn |
| chr12 | 102097529 | 102097530 | Hs06981209_cn |
| chr12 | 102105668 | 102105669 | Hs04412303_cn |
| chr13 | 40089549 | 40089550 | Hs03853267_cn |
| chr13 | 93444276 | 93444277 | Hs04432382 |
| chr13 | 93460071 | 93460072 | Hs04432043 |
| chr14 | 24519089 | 24519090 | Hs03883350 |
| chr14 | 24534221 | 24534222 | Hs01939905 |
| chr14 | 28522635 | 28522636 | CusTaq2CXLJH4P_cn |
| chr14 | 37916895 | 37916896 | Hs07055190_cn |
| chr14 | 37977977 | 37977978 | Hs07044926_cn |
| chr14 | 38014166 | 38014167 | Hs07086625_cn |
| chr14 | 38021288 | 38021289 | Hs07075472_cn |
| chr14 | 96763309 | 96763310 | Hs05318569_cn |
| chr14 | 96772014 | 96772015 | Hs00982344_cn |
| chr14 | 99641385 | 99641386 | Hs00596122_cn |
| chr14 | 100734909 | 100734910 | Hs03875129 |
| chr14 | 100765197 | 100765198 | Hs01931607 |
| chr14 | 100795059 | 100795060 | Hs00201515 |
| chr14 | 101000582 | 101000583 | Hs03874127_cn |
| chr14 | 101005643 | 101005644 | Hs01983727_cn |
| chr14 | 102021598 | 102021599 | Hs03877829_cn |
| chr14 | 102025461 | 102025462 | Hs03890390_cn |
| chr14 | 102737644 | 102737645 | Hs04443274_cn |
| chr14 | 102744822 | 102744823 | Hs04436664_cn |
| chr14 | 102974514 | 102974515 | Hs03874565_cn |
| chr14 | 104035624 | 104035625 | Hs07076467 |
| chr14 | 104089093 | 104089094 | Hs07094555 |
| chr14 | 104134199 | 104134200 | Hs07101222 |
| chr15 | 20194087 | 20194088 | Hs04444017 |
| chr15 | 25578159 | 25578160 | Hs03899505_cn |
| chr15 | 25580751 | 25580752 | CusTaq3CX20SJR_cn |
| chr15 | 25739587 | 25739588 | Hs03895201_cn |
| chr15 | 26170697 | 26170698 | Hs03899220_cn |
| chr15 | 26218978 | 26218979 | Hs07535627_cn |
| chr15 | 26566910 | 26566911 | Hs05379477_cn |
| chr15 | 26758634 | 26758635 | Hs05357961_cn |
| chr15 | 27186676 | 27186677 | Hs05354636_cn |
| chr15 | 27215751 | 27215752 | Hs05352889_cn |
| chr15 | 28430324 | 28430325 | Hs03904620_cn |
| chr15 | 28464592 | 28464593 | Hs03900299_cn |
| chr15 | 28510861 | 28510862 | Hs00790698_cn |
| chr15 | 30008107 | 30008108 | Hs03905821_cn |
| chr15 | 30028029 | 30028030 | Hs03894282_cn |
| chr15 | 31233791 | 31233792 | Hs01761674_cn |
| chr15 | 31418708 | 31418709 | Hs03907602_cn |
| chr15 | 31523604 | 31523605 | Hs05345027_cn |
| chr15 | 31779480 | 31779481 | Hs01740084_cn |
| chr15 | 31792000 | 31792001 | Hs03903842 |
| chr15 | 31807369 | 31807370 | Hs03898720 |
| chr15 | 31819397 | 31819398 | Hs01183107_cn |
| chr15 | 40565562 | 40565563 | Hs01801490_cn |
| chr15 | 40569495 | 40569496 | Hs03050146_cn |
| chr15 | 40574016 | 40574017 | Hs03915257 |
| chr15 | 40600033 | 40600034 | Hs02747689 |
| chr15 | 40631492 | 40631493 | Hs05348776 |
| chr15 | 42140352 | 42140353 | Hs01736986_cn |
| chr15 | 42220283 | 42220284 | Hs05327333_cn |
| chr15 | 42278083 | 42278084 | Hs07457532_cn |
| chr15 | 56246674 | 56246675 | Hs05388304_cn |
| chr15 | 56258673 | 56258674 | Hs02776763_cn |
| chr16 | 2137638 | 2137639 | Hs03948922_cn |
| chr16 | 2139578 | 2139579 | Hs01690407_cn |
| chr16 | 83908973 | 83908974 | Hs03924139_cn |
| chr16 | 83927884 | 83927885 | Hs03920294_cn |
| chr17 | 14133533 | 14133534 | Hs05489546_cn |
| chr17 | 15285417 | 15285418 | Hs05479141_cn |
| chr19 | 23823676 | 23823677 | Hs07158898_cn |
| chr19 | 23847358 | 23847359 | Hs07130588_cn |
| chr19 | 43260846 | 43260847 | Hs04483050_cn |
| chr19 | 52919934 | 52919935 | Hs01762991_cn |
| chr19 | 52961357 | 52961358 | Hs04015789_cn |
| chr20 | 8654182 | 8654183 | Hs07182273_cn |
| chr20 | 8655323 | 8655324 | Hs07214628_cn |
| chr20 | 8656129 | 8656130 | Hs07196671 |
| chr20 | 8662295 | 8662296 | Hs07181996 |
| chr20 | 32267585 | 32267586 | Hs03035919 |
| chr20 | 32324773 | 32324774 | Hs04040566 |
| chr20 | 32380921 | 32380922 | Hs07167677 |
| chr20 | 35244629 | 35244630 | Hs07189989_cn |
| chr20 | 35286976 | 35286977 | Hs07187468 |
| chr20 | 35339976 | 35339977 | Hs07195828 |
| chr20 | 35392781 | 35392782 | Hs07216584 |
| chr20 | 57246270 | 57246271 | Hs00451592_cn |
| chr20 | 57276159 | 57276160 | Hs02247879_cn |
| chr20 | 57283659 | 57283660 | Hs07195366_cn |
| chrX | 140316814 | 140316815 | Hs04119700_cn |
| chrX | 140348402 | 140348403 | Hs04105155_cn |
| chrX | 140394910 | 140394911 | Hs04123806_cn |
| chrX | 140450224 | 140450225 | Hs04514589_cn |
| chrX | 140560608 | 140560609 | Hs04117605_cn |
| chrX | 140711967 | 140711968 | Hs04108237 |
| chrX | 140730389 | 140730390 | Hs04114029 |
| chrX | 147283785 | 147283786 | Hs05619718 |
| chrX | 147557625 | 147557626 | Hs05666138 |
| chrX | 147831902 | 147831903 | Hs05592380 |
| chrX | 148101715 | 148101716 | Hs05606186 |
| chrX | 148379988 | 148379989 | Hs05667154 |
| chrX | 148892085 | 148892086 | Hs04109160_cn |
| chrX | 148999489 | 148999490 | Hs04513800_cn |
| chrX | 149014384 | 149014385 | Hs02798232_cn |
| chrX | 153195418 | 153195419 | Hs02879994_cn |
| chrX | 153200970 | 153200971 | Hs01730847_cn |

TABLE 8
153 CNVs in subjects with autism in Utah families

| | | | | | | | | | Custom |
|---|---|---|---|---|---|---|---|---|---|
| | | | | | | | | | iSelect |
| | | | | ACRD | | Gain/ | | | Array |
| No. | Chrom | Start (hg19) | End (hg19) | Published? | Ref. No. | Loss | Size (bp) | Gene | Probes |
| 1 | chr1 | 4737693 | 4746636 | N | | Loss | 8943 | AJAP1 | 20 |
| 2 | chr1 | 10624023 | 10627542 | N | | Loss | 3519 | PEX14 | 14 |
| 3 | chr1 | 145714421 | 146101228 | N | | Gain | 386807 | more than 10 genes | 20 |
| 4 | chr1 | 169704308 | 169732211 | N | | Loss | 27903 | C1orf112 | 20 |
| 5 | chr1 | 179456385 | 179472635 | N | | Loss | 16250 | C1orf125/ | 20 |
| | | | | | | | | DKFZp434N1720 | |
| 6 | chr1 | 204193679 | 204209979 | N | | Loss | 16300 | PLEKHA6 | 20 |
| 7 | chr1 | 215858193 | 215861879 | Y | 4 | Loss | 3686 | USH2A | 19 |
| 8 | chr1 | 225508461 | 225511454 | N | | Loss | 2993 | DNAH14 | 14 |
| 9 | chr1 | 228848896 | 228853665 | N | | Loss | 4769 | 5′ of RHOU | 11 |
| 10 | chr1 | 237993724 | 237995299 | N | | Loss | 1575 | RYR2 | 15 |
| 11 | chr1 | 243860912 | 243861049 | N | | Loss | 137 | AKT3 | 10 |
| 12 | chr2 | 12685369 | 12693172 | N | | Loss | 7803 | AK001558 | 16 |
| 13 | chr2 | 32982548 | 33050816 | Y | 2, 5 | Gain | 68268 | TTC27, AK095182 | 15 |
| 14 | chr2 | 37904904 | 37909117 | N | | Gain | 4213 | 5′ of CDC42EP3 | 19 |
| 15 | chr2 | 45997209 | 45997519 | N | | Loss | 310 | PRKCE | 11 |
| 16* | chr2 | 51272055 | 51336043 | Y | 2, 4 | Loss | 63988 | 5′ of NRXN1 (10 kb) | 83 |
| 17 | chr2 | 52420563 | 52584090 | N | | Loss | 163527 | 5′ of NRXN1 (1 Mb) | 20 |
| 18 | chr2 | 58346718 | 58349248 | Y | 2 | Loss | 2530 | VRK2 | 12 |
| 19 | chr2 | 62195814 | 62230970 | N | | Loss | 35156 | COMMD1, CR603473 | 20 |
| 20 | chr2 | 75014711 | 75044204 | N | | Loss | 29493 | 5′ of HK2 | 20 |
| 21 | chr2 | 79330766 | 79342811 | N | | Gain | 12045 | 5′ of REG1B, 5′ of | 17 |
| | | | | | | | | REG1A | |
| 22 | chr2 | 120130796 | 120145728 | N | | Loss | 14932 | 5′ of C2orf76, 5′ of | 20 |
| | | | | | | | | TMEM37 | |
| 23 | chr2 | 236424336 | 236465062 | N | | Loss | 40726 | AGAP1 | 20 |
| 24 | chr3 | 6724453 | 7046515 | N | | Gain | 322062 | AF279782, GRM7 | 20 |
| 25 | chr3 | 12387768 | 12393125 | N | | Loss | 5357 | PPARG | 20 |
| 26* | chr3 | 21731567 | 21734331 | N | | Gain | 2764 | ZNF385D | 14 |
| 27 | chr3 | 57051604 | 57053353 | N | | Gain | 1749 | ARHGEF3 | 13 |
| 28 | chr3 | 60774451 | 60777932 | Y | 3 | Gain | 3481 | FHIT | 16 |
| 29 | chr3 | 63962828 | 63964474 | N | | Loss | 1646 | ATXN7 | 13 |
| 30 | chr3 | 74566042 | 74584605 | N | | Loss | 18563 | CNTN3 | 20 |
| 31 | chr3 | 171090367 | 171092891 | N | | Gain | 2524 | TNIK | 16 |
| 32 | chr3 | 172596081 | 172617355 | N | | Gain | 21274 | SPATA16 | 20 |
| 33 | chr4 | 58811798 | 58816810 | N | | Loss | 5012 | 3′ of BC034799 (480 | 14 |
| | | | | | | | | kb) | |
| 34 | chr4 | 80865807 | 80887173 | N | | Loss | 21366 | ANTXR2/ | 17 |
| | | | | | | | | DKFZp667K1925 | |
| 35 | chr4 | 101551216 | 101616281 | N | | Loss | 65065 | 5′ of EMCN (200 kb) | 20 |
| 36 | chr4 | 134924034 | 135188390 | N | | Loss | 264356 | PABPC4L | 20 |
| 37 | chr4 | 185734577 | 185740215 | N | | Loss | 5638 | ACSL1 | 18 |
| 38 | chr4 | 189084983 | 189117429 | N | | Loss | 32446 | 3′ of TRIML1 | 20 |
| 39 | chr5 | 20436884 | 20449034 | N | | Loss | 12150 | CDH18 | 20 |
| 40 | chr5 | 58469036 | 58470270 | N | | Loss | 1234 | PDE4D | 12 |
| 41 | chr5 | 99634772 | 99682698 | N | | Loss | 47926 | 5′ of FAM174A (190 | 20 |
| | | | | | | | | kb) | |
| 42 | chr5 | 132621489 | 132630849 | Y | 2,4 | Gain | 9360 | FSTL4 | 20 |
| 43 | chr5 | 142599442 | 142602063 | N | | Loss | 2621 | ARHGAP26/KIAA0621 | 14 |
| 44 | chr5 | 151582812 | 151583410 | N | | Loss | 598 | AK001582 | 12 |
| 45 | chr6 | 7425246 | 7464367 | N | | Gain | 39121 | 3′ of RIOK1 | 20 |
| 46 | chr6 | 10856101 | 10872458 | N | | Loss | 16357 | 3′ of TMEM14B and | 20 |
| | | | | | | | | GCM2, 5′ of MAK and | |
| | | | | | | | | SYCP2L | |
| 47 | chr6 | 42126761 | 42128299 | N | | Loss | 1538 | GUCA1A | 16 |
| 48 | chr6 | 44113916 | 44180221 | N | | Loss | 66305 | CAPN11, TMEM63B | 20 |
| 49 | chr6 | 47864831 | 49244526 | N | | Loss | 1379695 | C6orf138 | 25 |
| 50 | chr6 | 53856580 | 53864523 | N | | Loss | 7943 | AK056584 | 19 |
| 51 | chr6 | 62443739 | 62462295 | N | | Loss | 18556 | KHDRBS2 | 17 |
| 52 | chr6 | 119419595 | 119427038 | Y | 2 | Loss | 7443 | FAM184A | 18 |
| 53 | chr6 | 123893763 | 123897553 | N | | Loss | 3790 | TRDN | 14 |
| 54 | chr6 | 139985775 | 140128887 | N | | Gain | 143112 | BC039503 | 20 |
| 55 | chr6 | 147588752 | 147664671 | Y | 2 | Gain | 75919 | STXBP5 | 20 |
| 56 | chr6 | 161189018 | 161218651 | N | | Loss | 29633 | 3′ of PLG | 20 |
| 57 | chr7 | 6838712 | 6864071 | N | | Loss | 25359 | C7orf28B | 15 |
| 58 | chr7 | 11782637 | 11783917 | Y | 4 | Loss | 1280 | THSD7A | 12 |
| 59 | chr7 | 13962113 | 13962620 | Y | 2 | Loss | 507 | ETV1 | 11 |
| 60 | chr7 | 71597328 | 71603027 | N | | Gain | 5699 | CALN1 | 14 |
| 61 | chr7 | 105285949 | 105321353 | N | | Loss | 35404 | ATXN7L1 | 20 |
| 62 | chr7 | 124546250 | 124580202 | Y | 4 | Loss | 33952 | POT1, hypothetical | 20 |
| | | | | | | | | proteins | |
| 63 | chr8 | 3160739 | 3160885 | N | | Loss | 146 | CSMD1/K1AA1890 | 10 |
| 64 | chr8 | 3169351 | 3169808 | N | | Loss | 457 | CSMD1/K1AA1890 | 11 |
| 65 | chr8 | 3479586 | 3480400 | N | | Loss | 814 | CSMD1 | 12 |
| 66 | chr8 | 4907673 | 4911422 | N | | Loss | 3749 | 5′ of CSMD1 60 kb) | 20 |
| 67 | chr8 | 31977229 | 31989597 | N | | Loss | 12368 | NRG1 | 20 |
| 68 | chr8 | 52261992 | 52265315 | N | | Loss | 3323 | PXDNL | 15 |
| 69 | chr8 | 84323466 | 84337983 | N | | Loss | 14517 | 3′ of BC038578 | 20 |
| 70 | chr8 | 85281895 | 85304198 | N | | Loss | 22303 | RALYL | 20 |
| 71 | chr8 | 86471729 | 86553130 | N | | Gain | 81401 | 3′ of REXO1L1 | 20 |
| 72 | chr8 | 100402969 | 100406592 | N | | Loss | 3623 | VPS13B | 10 |
| 73 | chr9 | 7036350 | 7051859 | N | | Loss | 15509 | JMJD2C | 20 |
| 74 | chr9 | 28027694 | 28039222 | N | | Gain | 11528 | LINGO2 | 20 |
| 75 | chr9 | 28190069 | 28347679 | N | | Loss | 157610 | LINGO2 | 20 |
| 76 | chr9 | 75206337 | 75207666 | N | | Gain | 1329 | TMC1 | 11 |
| 77 | chr9 | 116468123 | 116631674 | N | | Gain | 163551 | 5′ of ZNF618 (5 kb) | 12 |
| 78 | chr9 | 139083019 | 139113146 | N | | Gain | 30127 | LHX3, QSOX2 | 20 |
| 79 | chr10 | 27361202 | 27381349 | N | | Loss | 20147 | ANKRD26 | 20 |
| 80 | chr10 | 33217225 | 33222978 | N | | Loss | 5753 | ITGB1 | 11 |
| 81 | chr10 | 38914665 | 42953131 | N | | Loss | 4038466 | AK131313, BC039000 | 20 |
| 82 | chr10 | 52133698 | 52232708 | Y | 3 | Gain | 99010 | SGMS1/SMS1 | 20 |
| 83 | chr10 | 60793303 | 60857532 | Y | 3 | Gain | 64229 | 5′ of PHYHIPL (80 kb) | 20 |
| 84 | chr10 | 68350062 | 68375800 | N | | Loss | 25738 | CTNNA3 | 20 |
| 85 | chr10 | 81032555 | 81037800 | N | | Loss | 5245 | ZMIZ1 | 14 |
| 86 | chr10 | 83893626 | 84175018 | N | | Loss | 281392 | NRG3 | 13 |
| 87 | chr10 | 86939018 | 86970632 | N | | Loss | 31614 | AK097624 | 20 |
| 88 | chr10 | 89720106 | 89723874 | N | | Loss | 3768 | PTEN | 12 |
| 89 | chr10 | 91210650 | 91217984 | N | | Loss | 7334 | SLC16Al2 | 19 |
| 90 | chr10 | 92274764 | 92289762 | Y | 2 | Loss | 14998 | 3′ of BC037970 | 15 |
| 91 | chr11 | 7488341 | 7489819 | N | | Gain | 1478 | SYT9, AK128569 | 16 |
| 92 | chr11 | 12002139 | 12007077 | N | | Gain | 4938 | DKK3 | 20 |
| 93 | chr11 | 12374189 | 12374712 | N | | Loss | 523 | MICALCL | 11 |
| 94 | chr11 | 16569019 | 16576640 | N | | Loss | 7621 | SOX6/DKFZp434N1217 | 12 |
| 95 | chr11 | 31000774 | 31000929 | N | | Gain | 155 | DCDC5/KIAA1493 | 10 |
| 96 | chr11 | 60228735 | 60229382 | N | | Loss | 647 | MS4A1 | 11 |
| 97 | chr11 | 98148399 | 98212796 | N | | Gain | 64397 | 5′ of CNTN5 (700 kb) | 20 |
| 98 | chr11 | 100817655 | 100820663 | N | | Loss | 3008 | FLJ32810 | 14 |
| 99 | chr11 | 131405729 | 131406206 | N | | Gain | 477 | NTM, AK128059 | 11 |
| 100 | chr12 | 60173356 | 60173878 | Y | 4 | Gain | 522 | SLC16A7/MCT2 | 13 |
| 101 | chr12 | 73062598 | 73088289 | Y | 2 | Loss | 25691 | 3′ of TRHDE | 20 |
| 102 | chr12 | 75547922 | 75572356 | N | | Loss | 24434 | KCNC2 | 20 |
| 103 | chr12 | 80880491 | 80895554 | N | | Loss | 15063 | PTPRQ | 20 |
| 104 | chr12 | 80988331 | 81019079 | N | | Loss | 30748 | PTPRQ | 20 |
| 105 | chr12 | 81618586 | 81626675 | N | | Loss | 8089 | ACSS3 | 17 |
| 106 | chr12 | 97870273 | 97875696 | N | | Loss | 5423 | NCRMS/AK056164 | 20 |
| 107 | chr12 | 102097012 | 102106306 | N | | Loss | 9294 | CHPT1 | 13 |
| 108 | chr12 | 127308503 | 127315005 | Y, small | 4 | Loss | 6502 | between BC069215 | 19 |
| | | | | overlap | | | | and BC037858 | |
| 109 | chr13 | 40087689 | 40088007 | N | | Loss | 318 | LHFP | 12 |
| 110 | chr13 | 49284461 | 49343043 | N | | Gain | 58582 | 3′ of CYSLTR2 | 20 |
| 111 | chr13 | 50163809 | 50179454 | N | | Loss | 15645 | 5′ of RCBTB1 | 17 |
| 112 | chr13 | 93448487 | 93461603 | N | | Loss | 13116 | GPC5 | 17 |
| 113 | chr13 | 94357235 | 94369759 | N | | Loss | 12524 | GPC6 | 20 |
| 114 | chr14 | 23862374 | 23888040 | N | | Loss | 25666 | MYH6, MYH7, | 20 |
| | | | | | | | | MIR208B | |
| 115 | chr14 | 28506099 | 28520243 | N | | Loss | 14144 | between BC148262 | 20 |
| | | | | | | | | and CR597916 | |
| 116 | chr14 | 32904231 | 32909169 | N | | Gain | 4938 | AKAP6 | 20 |
| 117 | chr14 | 33859159 | 33860185 | N | | Gain | 1026 | NPAS3 | 11 |
| 118 | chr14 | 37928753 | 37948391 | N | | Loss | 19638 | MIPOL1 | 15 |
| 119 | chr14 | 68068610 | 68071772 | N | | Loss | 3162 | 5′ of PIGH | 15 |
| 120 | chr15 | 33605301 | 33617521 | N | | Gain | 12220 | RYR3 | 20 |
| 121 | chr15 | 47518807 | 47527672 | N | | Loss | 8865 | SEMA6D | 16 |
| 122 | chr15 | 58851369 | 58853307 | N | | Gain | 1938 | LIPC | 14 |
| 123 | chr15 | 60074956 | 60103803 | Y | 5 | Loss | 28847 | 5′ of BNIP2 (90 kb) | 20 |
| 124 | chr15 | 66521832 | 66524433 | N | | Loss | 2601 | MEGF11 | 17 |
| 125 | chr15 | 87830530 | 87870489 | N | | Loss | 39959 | between AGBL1, and | 20 |
| | | | | | | | | TMEM83, NTRK3 | |
| 126 | chr16 | 16245729 | 16256767 | N | | Loss | 11038 | ABCC6, MRP6 | 34 |
| 127 | chr16 | 21363810 | 21602618 | N | | Loss | 238808 | More than 10 genes | 25 |
| 128 | chr16 | 82446255 | 82711504 | Y | 5 | Gain | 265249 | CDH13 | 24 |
| 129 | chr16 | 83909041 | 83926368 | N | | Loss | 17327 | 5′ of MLYCD, 3′ of | 20 |
| | | | | | | | | HSBP1 | |
| 130 | chr17 | 4007594 | 4324408 | Y | 4 | Gain | 316814 | ZZEF1, KIAA0399, | 20 |
| | | | | | | | | CYB5D2, ANKFY1, | |
| | | | | | | | | UBE2G1, SPNS3 | |
| 131** | chr17 | 21556170 | 25363654 | N | | Loss | 3807484 | BC070367, FAM27L, | 20 |
| | | | | | | | | BC039120, CR592140, | |
| | | | | | | | | CR592128 | |
| 132 | chr17 | 39211908 | 39221312 | N | | Loss | 9404 | KRTAP2-4 | 15 |
| 133 | chr17 | 64258845 | 64259329 | N | | Loss | 484 | 5′ of APOH and 5′ of | 11 |
| | | | | | | | | PRKCA | |
| 134 | chr18 | 30037470 | 30037675 | N | | Loss | 205 | FAM59A | 10 |
| 135 | chr20 | 4234781 | 4238447 | N | | Gain | 3666 | 5′ of ADRA1D | 16 |
| 136 | chr20 | 6013320 | 6017259 | N | | Loss | 3939 | CRLS1/DKFZp762C112 | 14 |
| 137 | chr20 | 15755244 | 15765167 | N | | Loss | 9923 | MACROD2 | 20 |
| 138 | chr20 | 47337049 | 47341312 | N | | Gain | 4263 | PREX1 | 14 |
| 139 | chr20 | 49132410 | 49132637 | N | | Loss | 227 | PTPN1 | 10 |
| 140 | chr20 | 56248075 | 56252910 | N | | Loss | 4835 | PMEPA1 | 20 |
| 141 | chr21 | 17311697 | 17435462 | N | | Loss | 123765 | 5′ of C21orf34, 3′ of | 20 |
| | | | | | | | | USP25 | |
| 142 | chr21 | 42855515 | 42855647 | Y | 1 | Gain | 132 | TMPRSS2 | 10 |
| 143 | chr22 | 30731066 | 30731540 | N | | Gain | 474 | SF3A1 | 10 |
| 144 | chr22 | 33459104 | 33470309 | N | | Loss | 11205 | 5′ of SYN3 | 20 |
| 145 | chr22 | 39515118 | 39525791 | N | | Loss | 10673 | 3 of APOBEC3H, 3′ of | 20 |
| | | | | | | | | CBX7 | |
| 146 | chr22 | 44251958 | 44257056 | N | | Loss | 5098 | SULT4A1/SULTX3 | 19 |
| 147 | chr22 | 44641315 | 44641594 | N | | Gain | 279 | KIAA1644 | 10 |
| 148 | chr22 | 51055900 | 51234443 | Y | 4 | Gain | 178543 | ARSA, SHANK3, | 10 |
| | | | | | | | | BC050343, ACR, | |
| | | | | | | | | MGC70863, RABL2B | |
| 149 | chrX | 3206732 | 3216695 | N | | Loss | 9963 | 3′ of MXRA5, ARSF | 19 |
| 150 | chrX | 57285994 | 57291268 | N | | Gain | 5274 | 5′ of FAAH2 | 11 |
| 151 | chrX | 133460586 | 133466162 | N | | Loss | 5576 | 5′ of PHF6 | 11 |
| 152 | chrX | 142769032 | 142781735 | N | | Loss | 12703 | 5′ of SLITRK4, 3′ of | 15 |
| | | | | | | | | SPANXN2 | |
| 153 | chrX | 151041009 | 151042244 | N | | Loss | 1235 | 5′ of MAGEA4 | 12 |
| | | | | | | | | | Total = |
| | | | | | | | | | 2,642 |
| | | | | | | | | | Probes |

References:
1. Jacquemont et al., 2006
2. AGP, 2007
3. Sebat et al., 2007
4. Marshall et al., 2008
5. Christian et al., 2008
*Nos 16 & 26: includes overlapping literature CNVs
**No. 131: Much of this region spans the centromere and is heterochromatic

TABLE 9
185 CNVs reportedly associated with ASD from published studies

| | | | Custom |
|---|---|---|---|
| | | CNV Origin | iSelect |
| | | CHOP | Array |
| No. | CNV Regions (hg19, GRCh37) | Literature | Probes |
| 1 | chr1: 146626687-146641912 | CHOP_CNV | 208 |
| 2 | chr1: 146644352-146646782 | CHOP_CNV | 208 |
| 3 | chr1: 146649431-146651526 | CHOP_CNV | 208 |
| 4 | chr1: 146655885-146661221 | CHOP_CNV | 208 |
| 5 | chr1: 146714336-146767441 | CHOP_CNV | 208 |
| 6 | chr1: 147013183-147042947 | CHOP_CNV | 208 |
| 7 | chr1: 147119170-147142612 | CHOP_CNV | 208 |
| 8 | chr1: 147191843-147211176 | CHOP_CNV | 208 |
| 9 | chr1: 147228333-147245482 | CHOP_CNV | 208 |
| 10 | chr1: 152538131-152539246 | CHOP_CNV | 22 |
| 11 | chr1: 152551861-152552978 | CHOP_CNV | 22 |
| 12 | chr1: 176233934-176277050 | CHOP_CNV | 20 |
| 13 | chr2: 13202218-13248445 | CHOP_CNV | 20 |
| 14 | chr2: 37208154-37311483 | CHOP_CNV | 20 |
| 15 | chr2: 50147489-51240182 | CHOP_CNV | 84 |
| 16 | chr2: 51267143-51294094 | CHOP_CNV | 62 |
| 17 | chr2: 78414693-78457739 | CHOP_CNV | 20 |
| 18 | chr2: 99858712-99871568 | CHOP_CNV | 17 |
| 19 | chr2: 237821591-237832364 | CHOP_CNV | 94 |
| 20 | chr3: 1940192-1940920 | CHOP_CNV | 10 |
| 21 | chr3: 2573150-2573529 | CHOP_CNV | 11 |
| 22 | chr3: 4224733-4261302 | CHOP_CNV | 20 |
| 23 | chr3: 31702318-32023236 | CHOP_CNV | 20 |
| 24 | chr3: 37903670-38025958 | CHOP_CNV | 20 |
| 25 | chr3: 121343502-121387782 | CHOP_CNV | 20 |
| 26 | chr3: 172231370-173116242 | CHOP_CNV | 116 |
| 27 | chr3: 173116245-173254086 | CHOP_CNV | 100 |
| 28 | chr3: 173271686-173289279 | CHOP_CNV | 100 |
| 29 | chr3: 174001117-174885989 | CHOP_CNV | 100 |
| 30 | chr4: 13656804-13932850 | CHOP_CNV | 20 |
| 31 | chr4: 73756500-73905356 | CHOP_CNV | 60 |
| 32 | chr4: 73920417-73935470 | CHOP_CNV | 60 |
| 33 | chr4: 73940504-74124500 | CHOP_CNV | 60 |
| 34 | chr4: 144627954-144635127 | CHOP_CNV | 11 |
| 35 | chr5: 118229547-118343923 | CHOP_CNV | 100 |
| 36 | chr5: 118407187-118469872 | CHOP_CNV | 100 |
| 37 | chr5: 118478541-118584821 | CHOP_CNV | 100 |
| 38 | chr5: 118604420-118730292 | CHOP_CNV | 100 |
| 39 | chr5: 118730295-118856171 | CHOP_CNV | 100 |
| 40 | chr6: 39071841-39082863 | CHOP_CNV | 20 |
| 41 | chr6: 69235102-69237305 | CHOP_CNV | 10 |
| 42 | chr6: 122793063-123047516 | CHOP_CNV | 34 |
| 43 | chr6: 127440049-127518908 | CHOP_CNV | 20 |
| 44 | chr6: 135818945-136037191 | CHOP_CNV | 20 |
| 45 | chr6: 162664588-162667009 | CHOP_CNV | 31 |
| 46 | chr6: 168349013-168596249 | CHOP_CNV | 20 |
| 47 | chr7: 2649899-2654358 | CHOP_CNV | 20 |
| 48 | chr7: 32700564-32804186 | CHOP_CNV | 20 |
| 49 | chr7: 69064321-70257852 | CHOP_CNV | 23 |
| 50 | chr7: 111502940-111846460 | CHOP_CNV | 20 |
| 51 | chr7: 141695680-141806545 | CHOP_CNV | 20 |
| 52 | chr8: 43646415-43657436 | CHOP_CNV | 20 |
| 53 | chr8: 54858496-54907579 | CHOP_CNV | 20 |
| 54 | chr9: 116111824-116132133 | CHOP_CNV | 86 |
| 55 | chr9: 116135700-116139257 | CHOP_CNV | 85 |
| 56 | chr9: 119187508-120177315 | CHOP_CNV | 58 |
| 57 | chr9: 136501486-136524464 | CHOP_CNV | 37 |
| 58 | chr10: 87359313-87944322 | CHOP_CNV | 105 |
| 59 | chr10: 87951688-87959047 | CHOP_CNV | 79 |
| 60 | chr10: 88126251-88893189 | CHOP_CNV | 104 |
| 61 | chr10: 105353785-105615162 | CHOP_CNV | 20 |
| 62 | chr10: 118350491-118368684 | CHOP_CNV | 20 |
| 63 | chr12: 31409581-31410819 | CHOP_CNV | 13 |
| 64 | chr12: 53183470-53189890 | CHOP_CNV | 20 |
| 65 | chr12: 57345220-57352101 | CHOP_CNV | 20 |
| 66 | chr12: 71833814-71980084 | CHOP_CNV | 20 |
| 67 | chr13: 20977807-21100010 | CHOP_CNV | 20 |
| 68 | chr14: 94184645-94254764 | CHOP_CNV | 20 |
| 69 | chr15: 23686020-23692388 | CHOP_CNV | 19 |
| 70 | chr15: 24842742-24979665 | CHOP_CNV | 47 |
| 71 | chr15: 25101701-25223727 | CHOP_CNV | 53 |
| 72 | chr16: 16243423-16317335 | CHOP_CNV | 40 |
| 73 | chr16: 47276822-47330242 | CHOP_CNV | 20 |
| 74 | chr16: 70954495-71007921 | CHOP_CNV | 20 |
| 75 | chr16: 75572016-75590168 | CHOP_CNV | 20 |
| 76 | chr16: 84599210-84610700 | CHOP_CNV | 40 |
| 77 | chr17: 30819629-31203900 | CHOP_CNV | 20 |
| 78 | chr17: 64298927-64806860 | CHOP_CNV | 31 |
| 79 | chr18: 3498838-3880133 | CHOP_CNV | 20 |
| 80 | chr19: 22639351-22639555 | CHOP_CNV | 10 |
| 81 | chr19: 23835709-23870015 | CHOP_CNV | 38 |
| 82 | chr19: 23926161-23941637 | CHOP_CNV | 38 |
| 83 | chr19: 43225795-43440224 | CHOP_CNV | 20 |
| 84 | chr19: 52880583-52901119 | CHOP_CNV | 108 |
| 85 | chr19: 52901122-52909308 | CHOP_CNV | 108 |
| 86 | chr19: 52909311-52921656 | CHOP_CNV | 108 |
| 87 | chr19: 52932442-52934660 | CHOP_CNV | 108 |
| 88 | chr19: 52934663-52942694 | CHOP_CNV | 108 |
| 89 | chr19: 52956761-52961405 | CHOP_CNV | 108 |
| 90 | chr20: 8113297-8865545 | CHOP_CNV | 40 |
| 91 | chr20: 55993557-55997466 | CHOP_CNV | 33 |
| 92 | chr22: 21021266-21028944 | CHOP_CNV | 19 |
| 93 | chr22: 29999566-30094583 | CHOP_CNV | 20 |
| 94 | chrX: 6966962-7066187 | CHOP_CNV | 20 |
| 95 | chrX: 139998330-140335594 | CHOP_CNV | 71 |
| 96 | chrX: 140335597-140443613 | CHOP_CNV | 71 |
| 97 | chrX: 140590844-140672859 | CHOP_CNV | 71 |
| 98 | chrX: 140677836-140678897 | CHOP_CNV | 71 |
| 99 | chrX: 140713997-140714859 | CHOP_CNV | 71 |
| 100 | chrX: 148663310-148669114 | CHOP_CNV | 60 |
| 101 | chrX: 148676928-148678215 | CHOP_CNV | 60 |
| 102 | chrX: 148678218-148713566 | CHOP_CNV | 60 |
| 103 | chrX: 148858522-149097275 | CHOP_CNV | 60 |
| 104 | chrX: 154719774-154842595 | CHOP_CNV | 40 |
| 105 | chr1: 110230419-110236364 | Literature_CNV | 0 |
| 106 | chr1: 146555186-147779086 | Literature_CNV | 152 |
| 107 | chr1: 162573378-167543374 | Literature_CNV | 61 |
| 108 | chr1: 230111830-232145817 | Literature_CNV | 43 |
| 109 | chr2: 54076-1198908 | Literature_CNV | 23 |
| 110 | chr2: 17406571-18378433 | Literature_CNV | 21 |
| 111 | chr2: 32678416-33378738 | Literature_CNV | 40 |
| 112 | chr2: 45455651-45984915 | Literature_CNV | 31 |
| 113 | chr2: 50145644-51259671 | Literature_CNV | 84 |
| 114 | chr2: 51979551-52401447 | Literature_CNV | 40 |
| 115 | chr2: 57200002-61699998 | Literature_CNV | 98 |
| 116 | chr2: 62258231-63028717 | Literature_CNV | 48 |
| 117 | chr2: 115139568-115617934 | Literature_CNV | 20 |
| 118 | chr2: 162387215-162840241 | Literature_CNV | 20 |
| 119 | chr2: 198797484-209741388 | Literature_CNV | 119 |
| 120 | chr2: 236632457-238435065 | Literature_CNV | 101 |
| 121 | chr2: 238435068-242985349 | Literature_CNV | 125 |
| 122 | chr3: 2028902-2884398 | Literature_CNV | 31 |
| 123 | chr3: 11034422-11080933 | Literature_CNV | 20 |
| 124 | chr3: 67656832-68957204 | Literature_CNV | 24 |
| 125 | chr3: 100203669-100487283 | Literature_CNV | 20 |
| 126 | chr3: 143608410-144494785 | Literature_CNV | 20 |
| 127 | chr3: 195674002-197284998 | Literature_CNV | 27 |
| 128 | chr4: 154087652-172339893 | Literature_CNV | 191 |
| 129 | chr5: 176990003-180905258 | Literature_CNV | 42 |
| 130 | chr6: 13889303-15153950 | Literature_CNV | 24 |
| 131 | chr7: 23876-1297908 | Literature_CNV | 16 |
| 132 | chr7: 15386880-15538756 | Literature_CNV | 20 |
| 133 | chr7: 72576596-75922729 | Literature_CNV | 42 |
| 134 | chr7: 83144216-86082367 | Literature_CNV | 40 |
| 135 | chr7: 87999366-89294562 | Literature_CNV | 24 |
| 136 | chr7: 121210655-121381762 | Literature_CNV | 40 |
| 137 | chr7: 121755766-122152424 | Literature_CNV | 40 |
| 138 | chr7: 128907065-128998138 | Literature_CNV | 20 |
| 139 | chr7: 152589804-152616097 | Literature_CNV | 20 |
| 140 | chr8: 6264122-6506023 | Literature_CNV | 20 |
| 141 | chr8: 53271330-53555369 | Literature_CNV | 20 |
| 142 | chr9: 7735282-7770231 | Literature_CNV | 20 |
| 143 | chr9: 38027602-38298598 | Literature_CNV | 20 |
| 144 | chr9: 102472181-136065177 | Literature_CNV | 464 |
| 145 | chr10: 13049365-13367445 | Literature_CNV | 20 |
| 146 | chr10: 46269076-50892143 | Literature_CNV | 64 |
| 147 | chr10: 50892146-51450787 | Literature_CNV | 32 |
| 148 | chr10: 84158614-89685463 | Literature_CNV | 178 |
| 149 | chr11: 40329226-40653822 | Literature_CNV | 20 |
| 150 | chr13: 23604102-24794298 | Literature_CNV | 23 |
| 151 | chr13: 35516457-36246870 | Literature_CNV | 20 |
| 152 | chr13: 48083039-48475962 | Literature_CNV | 20 |
| 153 | chr13: 67572852-67762297 | Literature_CNV | 20 |
| 154 | chr15: 20266959-25480660 | Literature_CNV | 123 |
| 155 | chr15: 25582397-25684125 | Literature_CNV | 28 |
| 156 | chr15: 73090002-76507998 | Literature_CNV | 44 |
| 157 | chr15: 85105976-85708062 | Literature_CNV | 20 |
| 158 | chr16: 2097991-2138710 | Literature_CNV | 20 |
| 159 | chr16: 6052837-6260813 | Literature_CNV | 20 |
| 160 | chr16: 14982501-16482497 | Literature_CNV | 64 |
| 161 | chr16: 21534307-21901307 | Literature_CNV | 48 |
| 162 | chr16: 21901310-22703860 | Literature_CNV | 34 |
| 163 | chr16: 29671216-30173786 | Literature_CNV | 20 |
| 164 | chr16: 82195236-82722082 | Literature_CNV | 40 |
| 165 | chr17: 9964035-10361280 | Literature_CNV | 20 |
| 166 | chr17: 14139846-15282723 | Literature_CNV | 23 |
| 167 | chr17: 48646233-48704540 | Literature_CNV | 20 |
| 168 | chr18: 32073255-35145997 | Literature_CNV | 42 |
| 169 | chr19: 27896698-28805250 | Literature_CNV | 20 |
| 170 | chr20: 127914-419869 | Literature_CNV | 20 |
| 171 | chr20: 2837196-4006397 | Literature_CNV | 23 |
| 172 | chr20: 8044044-8527513 | Literature_CNV | 30 |
| 173 | chr20: 41602847-41867105 | Literature_CNV | 20 |
| 174 | chr21: 37412682-37622182 | Literature_CNV | 20 |
| 175 | chr22: 18640348-21461644 | Literature_CNV | 51 |
| 176 | chr22: 38368320-38380536 | Literature_CNV | 20 |
| 177 | chr22: 47956883-49122331 | Literature_CNV | 36 |
| 178 | chr22: 49405478-49971756 | Literature_CNV | 29 |
| 179 | chr22: 51113071-51171638 | Literature_CNV | 36 |
| 180 | chrX: 94421-5469456 | Literature_CNV | 78 |
| 181 | chrX: 5808084-5999993 | Literature_CNV | 20 |
| 182 | chrX: 28605682-29974014 | Literature_CNV | 25 |
| 183 | chrX: 53300002-53699998 | Literature_CNV | 20 |
| 184 | chrX: 70364712-70391048 | Literature_CNV | 20 |
| 185 | chrX: 153213010-153399998 | Literature_CNV | 40 |
| | | | Total = |
| | | | 4,492 |
| | | | probes* |

*Note that there is significant redundancy in this probe set, as many of the literature CNVs included on the array overlapped.

TABLE 10
25 CNVs identified from single nucleotide variants (SNVs) on custom array

| | | | | | Start | | |
|---|---|---|---|---|---|---|---|
| | | Gain or | Validation | | Coord. | End Coord. | |
| No. | CNV Source | Loss | Status | Chromosome | (hg19) | (hg19) | Gene(s) |
| 1 | SequenceSNP | Loss | PASS | chr7 | 93070811 | 93116320 | CALCR MIR653 MIR489 |
| 2 | SequenceSNP | Gain | PASS | chr14 | 100705631 | 100828134 | SLC25A29 YY1 MIR345 |
| | | | | | | | SLC25A47 WARS |
| 3 | SequenceSNP | Gain | PASS | chr14 | 102018946 | 102026138 | DIO3AS DIO3O5 |
| 4 | SequenceSNP | Loss | PASS | chr14 | 102729881 | 102749930 | MOK/RAGE |
| 5 | SequenceSNP | Gain | PASS | chr14 | 102973910 | 102975572 | ANKRD9 |
| 6 | SequenceSNP | Gain | PASS | chr15 | 25690465 | 26793077 | ATP10A MIR4715 |
| | | | | | | | GABRB3 LOC503519 |
| | | | | | | | LOC100128714 |
| 7 | SequenceSNP | Gain | PASS | chr15 | 27184517 | 27216737 | GABRA5 GABRG3 |
| 8 | SequenceSNP | Gain | PASS | chr15 | 28408312 | 28513763 | HERC2 |
| 9 | SequenceSNP | Loss | PASS | chr15 | 31092983 | 31369123 | FAN1 TRPM1 MTMR10 |
| | | | | | | | MIR211 TRPM1 |
| 10 | SequenceSNP | Gain/Loss | PASS | chr15 | 31776648 | 31822910 | OTUD7A |
| 11 | SequenceSNP | Gain | PASS | chr20 | 32210931 | 32441302 | NECAB3 CBFA2T2 E2F1 |
| | | | | | | | C20orf134 ZNF341 |
| | | | | | | | C20orf144 PXMP4 ZNF341 |
| | | | | | | | CHMP4B |
| 12 | SequenceSNP | Gain | No data | chr14 | 99640708 | 99642376 | BCL11B |
| 13 | SequenceSNP | Loss | FAIL | chr3 | 176755900 | 176782811 | TBL1XR1 |
| 14 | SequenceSNP | Gain | FAIL | chr7 | 100159979 | 100456457 | MOSPD3 TFR2 |
| | | | | | | | LOC100129845 GIGYF1 |
| | | | | | | | GNB2 LRCH4 ACTL6B |
| | | | | | | | FBXO24 PCOLCE AGFG2 |
| | | | | | | | SAP25 POP7 GIGF1 ZAN |
| | | | | | | | SLC12A9 EPHB4 |
| 15 | SequenceSNP | Gain/Loss | FAIL | chr7 | 149481075 | 149576256 | SSPO ATP6V0E2 ZNF862 |
| | | | | | | | LOC401431 |
| 16 | SequenceSNP | Gain | FAIL | chr14 | 24507010 | 24550497 | DHRS4L1 LRRC16B NRL |
| | | | | | | | CPNE6 |
| 17 | SequenceSNP | Loss | FAIL | chr14 | 96758018 | 96777946 | ATG2B |
| 18 | SequenceSNP | Gain | FAIL | chr14 | 100995537 | 101010301 | BEGAIN WDR25 |
| 19 | SequenceSNP | Gain | FAIL | chr14 | 103986349 | 104182224 | TRMT61A CKB TRMT61A |
| | | | | | | | BAG5 APOPT1 C14orf153 |
| | | | | | | | XRCC3 KLC1 ZFYVE21 |
| 20 | SequenceSNP | Gain | FAIL | chr15 | 30000877 | 30033536 | TJP1 |
| 21 | SequenceSNP | Gain | FAIL | chr15 | 40544493 | 40661306 | C15orf56 PAK6 PLCB2 |
| | | | | | | | C15orf52 DISP2 |
| 22 | SequenceSNP | Gain | FAIL | chr15 | 42139583 | 42302433 | JMJD7-PLA2G4B |
| | | | | | | | PLA2G4B SPTBN5 EHD4 |
| | | | | | | | PLA2G4E |
| 23 | SequenceSNP | Loss | FAIL | chr15 | 56243611 | 56258744 | NEDD4 |
| 24 | SequenceSNP | Gain | FAIL | chr20 | 35234192 | 35444437 | NDRG3 TGIF2-C20ORF24 |
| | | | | | | | C20orf24 SLA2 DSN1 |
| | | | | | | | KIAA0889 |
| 25 | SequenceSNP | Gain | FAIL | chr20 | 57268867 | 57290347 | NPEPL1 STX16-NPEPL1 |

### Example 2

### Design of a Custom Clinical Array

A custom clinical array was designed based on the results of the study described in Example 1. The study array used in Example 1 included about 10,000 probes for the regions being studied. Therefore, a custom array was specifically designed for clinical use to enhance coverage for the CNVs identified as associated with ASD. Custom probes for detection of other childhood developmental delay disorders were also included on the array as outlined in Table 11 below.

Table 11 below summarizes the custom probes designed for and included on the clinical array. The clinical array is based on the Affymetrix CytoScan-HD array and includes the 83,443 custom probes provided in the sequence listing and also described in Table 14. The 83,443 probes were added to the Affymetrix array to ensure sufficient coverage of all of the regions described in Tables 8 and 9, as well as to detect CNVs for the other disorders listed in Table 11.

TABLE 11
Summary of Custom Probes

| | | Custom CNV |
|---|---|---|
| Disorder | CNV source | Probes |
| Autism | Literature CNVs | 58950 |
| | Utah CNVs | 3691 |
| | CHOP CNVs | 2619 |
| Utah familial sequence variants | | |
| Rett syndrome | | 28 |
| Noonan/Costello/CFC syndromes | | 0 |
| Tuberous sclerosis | | 0 |
| ADHD | | 8764 |
| DD | | 9364 |
| Tourette syndrome | | 27 |
| Dyslexia | | 0 |
| | Total | 83443 |

A description of the custom probes as summarized in Table 11 is provided in Table 14. Table 14 provides the following information: The third column, labeled “hg19 Coordinates/Gene Name”, displays the genome coordinates (hg19) of the CNV for which each probe was designed. The second column, labeled “EXPOS” displays the nucleotide position within the chromosomal region shown in the third column that represents the center of the oligonucleotide probe. The oligonucleotides themselves are 25 nucleotides in length, so the center is nucleotide 13. The first column lists the SEQ ID NO for the oligonucleotide (DNA probe) which is provided in the sequence listing.

Tables 12 and 13 below list the CNVs identified in the study described in Example 1 (from Tables 3 and 4), and further include the SEQ ID NOs for the custom probes, where applicable. Since custom probes were only included on the array for some CNVs identified in Example 1, N/A is used to denote that no custom probes were used. Sequences of the custom probes are set forth in the sequence listing as SEQ ID NOs:1-83,443. As noted above, the positions of the probes are described in Table 14.

TABLE 12
Summary of Custom Probes for CNVs from Table 3

| | | | Custom Probe |
|---|---|---|---|
| No. | CNV Region - Replication Cohort | Gene/Region | SEQ ID NOs¹ |
| 1 | chr1: 145703115-145736438 | CD160, PDZK1 | N/A |
| 2 | chr1: 215854466-215861792 | USH2A | 27,988-28,001 |
| 3 | chr2: 51266798-51339236 | upstream of NRXN1 | 32,494-32,587 |
| 4 | chr3: 172591359-172604675 | downstream of SPATA16 | N/A |
| 5 | chr4: 189084240-189117031 | downstream of TRIML1 | N/A |
| 6 | chr6: 7461346-7470321 | between RIOK1 and DSP | 62,966-62,998 |
| 7 | chr6: 62426827-62472074 | KHDRBS2 | N/A |
| 8 | chr6: 147577803-147684318 | STXBP5 | N/A |
| 9 | chr7: 6870635-6871412 | upstream of CCZ1B | 69,319-69,561 |
| 10 | chr7: 93070811-93116320 | CALCR, MIR653, MIR489 | N/A |
| 11 | chr9: 28207468-28348133 | LINGO2 | N/A |
| 12 | chr9: 28354180-28354967 | LINGO2 (intron) | N/A |
| 13 | chr10: 83886963-83888343 | NRG3 (intron) | N/A |
| 14 | chr10: 92262627-92298079 | downstream of BC037970 | N/A |
| 15 | chr12: 102095178-102108946 | CHPT1 | 7410-7426 |
| 16 | chr13: 40089105-40090197 | LHFP (intron) | N/A |
| 17 | chr14: 100705631-100828134 | SLC25A29, YY1, MIR345, | N/A |
| | | SLC25A47, WARS | |
| 18 | chr14: 102018946-102026138 | DIO3AS, DIO3OS | N/A |
| 19 | chr14: 102729881-102749930 | MOK | N/A |
| 20 | chr14: 102973910-102975572 | ANKRD9 (RAGE) | N/A |
| 21 | chr15: 25690465-28513763 | ATP10A, GABRB3, | N/A |
| | | GABRA5, GABRG3, | |
| 22 | chr15: 31092983-31369123 | FAN1, MTMR10, MIR211, | N/A |
| | | TRPM1 | |
| 23 | chr15: 31776648-31822910 | OTUD7A | N/A |
| 24 | chr20: 32210931-32441302 | NECAB3, CBFA2T2, | N/A |
| | | C20orf144, NECAB3, | |

¹Custom probes were only included on the array for some CNVs.
N/A denotes that no custom probes were used.

TABLE 13
Summary of Custom Probes for CNVs from Table 4

| | | | Custom Probe |
|---|---|---|---|
| No. | Region of Highest Significance | Gene/Region | SEQ ID NOs¹ |
| 1 | chr1: 146656292-146707824 | FMO5 | N/A |
| 2 | chr2: 13203874-13209245 | upstream of LOC100506474 | 31,283-31,314 |
| 3 | chr2: 45489954-45492582 | between UNQ6975 and | N/A |
| | | SRBD1 | |
| 4 | chr2: 51237767-51245359 | NRXN1** | N/A |
| 5 | chr2: 62230970-62367720 | COMMD1 | 33,402-39,860 |
| 6 | chr2: 115133493-115140263 | between LOC440900 and | N/A |
| | | DPP10** | |
| 7 | chr3: 1937796-1941004 | between CNTN6 and | N/A |
| | | CNTN4** | |
| 8 | chr3: 67657429-68962928 | SUCLG2, FAM19A4, | N/A |
| | | FAM19A1 | |
| 9 | chr4: 73766964-73816870 | COX18, ANKRD17 | 51,803-52,100 |
| 10 | chr4: 171366005-171471530 | between AADAT** and | N/A |
| | | HSP90AA6P | |
| 11 | chr5: 118527524-118589485 | DMXL1, TNFAIP8 | 61,165-61,290 |
| 12 | chr6: 39069291-39072241 | SAYSD1 | 64,149-64,167 |
| 13 | chr8: 54855680-54912001 | RGS20, TCEA1 | N/A |
| 14 | chr10: 49370090-49471091 | FRMPD2P1, FRMPD2 | N/A |
| 15 | chr10: 50884949-50943185 | OGDHL, C10orf53 | N/A |
| 16 | chr12: 53177144-53180552 | between KRT76 and KRT3 | N/A |
| 17 | chr15: 20192970-20197164 | downstream of HERC2P3 | 12,508-12,563 |
| 18 | chr15: 25099351-25102073 | SNRPN** | N/A |
| 19 | chr15: 25099351-25102073 | SNRPN** | N/A |
| 20 | chr15: 25579767-25581658 | between SNORD109A and | N/A |
| | | UBE3A** | |
| 21 | chr15: 25582882-25662988 | UBE3A** | N/A |
| 22 | chr16: 21958486-22172866 | C16orf52, UQCRC2**, | N/A |
| | | PDZD9, VWA3A | |
| 23 | chr16: 29664753-30177298 | DOC2A**, ASPHD1, | N/A |
| | | LOC440356, TBX6, | |
| | | LOC100271831, PRRT2 | |
| | | CDIPT, QPRT, YPEL3, | |
| | | PPP4C, MAPK3**, SPN, | |
| | | MVP, FAM57B, ZG16, | |
| | | ALDOA, INO80E, SEZ6L2, | |
| | | TAOK2, KCTD13, MAZ, | |
| | | KIF22, GDPD3, C16orf92, | |
| | | C16orf53, TMEM219, | |
| | | C16orf54, HIRIP3 | |
| 24 | chr16: 82423855-82445055 | between MPHOSPH6 and | N/A |
| | | CDH13 | |
| 25 | chr17: 14132271-14133349 | between COX10 and | N/A |
| | | CDRT15 | |
| 26 | chr17: 14132271-15282708 | PMP22**, CDRT15, TEKT3, | N/A |
| | | MGC12916, CDRT7, | |
| | | HS3ST3B1 | |
| 27 | chr17: 14952999-15053648 | between CDRT7 and PMP22 | N/A |
| 28 | chr17: 15283960-15287134 | between TEKT3 and | N/A |
| | | FAM18B2-CDRT4 | |
| 29 | chr20: 8162278-8313229 | PLCB1** | N/A |
| 30 | chrX: 29944502-29987870 | IL1RAPL1** | N/A |
| 31 | chrX: 140329633-140348506 | SPANXC | N/A |
| 32 | chrX: 148882559-148886166 | MAGEA8 | N/A |

¹Custom probes were only included on the array for some CNVs.
N/A denotes that no custom probes were used.

### REFERENCES CITED

1. Rosenberg R E, Law J K, Yenokyan G, McGready J, Kaufmann W E, et al. (2009) Characteristics and Concordance of Autism Spectrum Disorders Among 277 Twin PairsAutism Characteristics and Discordance in Twins. Arch Pediatr Adolesc Med 163: 907-914. doi:10.1001/archpediatrics.2009.98.
2. Hallmayer J, Cleveland S, Torres A, Phillips J, Cohen B, et al. (2011) Genetic Heritability and Shared Environmental Factors Among Twin Pairs With Autism. Arch Gen Psychiatry 68: 1095-1102. doi:10.1001/archgenpsychiatry.2011.76.
3. Lichtenstein P, Carlström E, Rästam M, Gillberg C, Anckarsäter H (2010) The Genetics of Autism Spectrum Disorders and Related Neuropsychiatric Disorders in Childhood. Am J Psychiatry 167: 1357-1363. doi:10.1176/appi.ajp.2010.10020223.
4. Ronald A, Hoekstra R A (2011) Autism spectrum disorders and autistic traits: A decade of new twin studies. Am J Med Genet B Neuropsychiatr Genet 156B: 255-274. doi:10.1002/ajmg.b.31159.
5. International Molecular Genetic Study of Autism Consortium (IMGSAC) (1998) A Full Genome Screen for Autism with Evidence for Linkage to a Region on Chromosome 7q . Hum Mol Genet 7: 571-578. doi:10.1093/hmg/7.3.571.
6. International Molecular Genetic Study of Autism Consortium (IMGSAC) (2001) A Genomewide Screen for Autism: Strong Evidence for Linkage to Chromosomes 2q, 7q, and 16p. Am J Hum Genet 69: 570-581. doi:10.1086/323264.
7. Buxbaum J D, Silverman J, Keddache M, Smith C J, Hollander E, et al. (2003) Linkage analysis for autism in a subset families with obsessive-compulsive behaviors: Evidence for an autism susceptibility gene on chromosome 1 and further support for susceptibility genes on chromosome 6 and 19. Mol Psychiatry 9: 144-150.
   doi:10.1038/sj.mp.4001465.
8. Martin C L, Ledbetter D H (2007) Autism and cytogenetic abnormalities: solving autism one chromosome at a time. Curr Psychiatry Rep 9: 141-147.
9. Levy D, Ronemus M, Yamrom B, Lee Y, Leotta A, et al. (2011) Rare De Novo and Transmitted Copy-Number Variation in Autistic Spectrum Disorders. Neuron 70: 886-897. doi:10.1016/j.neuron.2011.05.015.
10. Betancur C (2011) Etiological heterogeneity in autism spectrum disorders: More than 100 genetic and genomic disorders and still counting. Brain Res 1380: 42-77.
   doi:10.1016/j.brainres.2010.11.078.
11. Sanders S J, Murtha M T, Gupta A R, Murdoch J D, Raubeson M J, et al. (2012) De novo mutations revealed by whole-exome sequencing are strongly associated with autism. Nature 485(7397):237-241. doi:10.1038/nature10945
12. lossifov I, Ronemus M, Levy D, Wang Z, Hakker I, et al. (2012) De Novo Gene Disruptions in Children on the Autistic Spectrum. Neuron 74: 285-299.
   doi:10.1016/j.neuron.2012.04.009.
13. Girirajan S, Brkanac Z, Coe B P, Baker C, Vives L, et al. (2011) Relative burden of large CNVs on a range of neurodevelopmental phenotypes. PLoS Genet 7:
   e1002334. doi:10.1371/joumal.pgen.1002334.
14. Sebat J, Lakshmi B, Malhotra D, Troge J, Lese-Martin C, et al. (2007) Strong Association of De Novo Copy Number Mutations with Autism. Science 316: 445-449. doi:10.1126/science.1138659.
15. Marshall C R, Noor A, Vincent J B, Lionel A C, Feuk L, et al. (2008) Structural Variation of Chromosomes in Autism Spectrum Disorder. Am J Hum Genet 82: 477-488. doi:10.1016/j.ajhg.2007.12.009.
16. Christian S L, Brune C W, Sudi J, Kumar R A, Liu S, et al. (2008) Novel Submicroscopic Chromosomal Abnormalities Detected in Autism Spectrum Disorder. Biol Psychiatry 63: 1111-1117. doi:10.1016/j.biopsych.2008.01.009.
17. Glessner J T, Wang K, Cai G, Korvatska O, Kim C E, et al. (2009) Autism genome-wide copy number variation reveals ubiquitin and neuronal genes. Nature 459: 569-573. doi:10.1038/nature07953.
18. Bucan M, Abrahams B S, Wang K, Glessner J T, Herman E I, et al. (2009) Genome-Wide Analyses of Exonic Copy Number Variants in a Family-Based Study Point to Novel Autism Susceptibility Genes. PLoS Genet 5: e1000536.
   doi:10.1371/journal.pgen.1000536.
19. Pinto D, Pagnamenta A T, Klei L, Anney R, Merico D, et al. (2010) Functional impact of global rare copy number variation in autism spectrum disorders. Nature 466: 368-372. doi:10.1038/nature09146.
20. Szatmari P, Paterson A D, Zwaigenbaum L, Roberts W, Brian J (2007) Mapping autism risk loci using genetic linkage and chromosomal rearrangements. Nat Genet 39: 319-328. doi:10.1038/ng1985.
21. Weiss L A, Shen Y, Korn J M, Arking D E, Miller D T, et al. (2008) Association between Microdeletion and Microduplication at 16p11.2 and Autism. N Engl J Med 358: 667-675. doi:10.1056/NEJMoa075974.
22. Morrow E M, Yoo S-Y, Flavell S W, Kim T-K, Lin Y, et al. (2008) Identifying Autism Loci and Genes by Tracing Recent Shared Ancestry. Science 321: 218 -223.
   doi:10.1126/science.1157657.
23. Jacquemont M-L, Sanlaville D, Redon R, Raoul O, Cormier-Daire V, et al. (2006) Array-based comparative genomic hybridisation identifies high frequency of cryptic chromosomal rearrangements in patients with syndromic autism spectrum disorders. J Med Genet 43: 843-849. doi:10.1136/jmg.2006.043166.
24. Shinawi M, Liu P, Kang S-H L, Shen J, Belmont J W, et al. (2010) Recurrent reciprocal 16p11.2 rearrangements associated with global developmental delay, behavioural problems, dysmorphism, epilepsy, and abnormal head size. J Med Genet 47: 332-341. doi:10.1136/jmg.2009.073015.
25. Shen Y, Dies K A, Holm I A, Bridgemohan C, Sobeih M M, et al. (2010) Clinical Genetic Testing for Patients With Autism Spectrum Disorders. Pediatrics 125: e727-e735. doi:10.1542/peds.2009-1684.
26. Fernandez B A, Roberts W, Chung B, Weksberg R, Meyn S, et al. (2010) Phenotypic spectrum associated with de novo and inherited deletions and duplications at 16p11.2 in individuals ascertained for diagnosis of autism spectrum disorder. J Med Genet 47: 195-203. doi:10.1136/jmg.2009.069369.
27. Lionel A C, Crosbie J, Barbosa N, Goodale T, Thiruvahindrapuram B, et al. (2011) Rare copy number variation discovery and cross-disorder comparisons identify risk genes for ADHD. Sci Transl Med 3: 95ra75. doi:10.1126/scitranslmed.3002464.
28. Sahoo T, Theisen A, Rosenfeld J A, Lamb A N, Ravnan J B, et al. (2011) Copy number variants of schizophrenia susceptibility loci are associated with a spectrum of speech and developmental delays and behavior problems. Genet Med 13: 868-880. doi:10.1097/GIM.0b013e3182217a06.
29. Kirov G, Pocklington A J, Holmans P, Ivanov D, Ikeda M, et al. (2012) De novo CNV analysis implicates specific abnormalities of postsynaptic signalling complexes in the pathogenesis of schizophrenia. Mol Psychiatry 17: 142-153.
   doi:10.1038/mp.2011.154.
30. Manning M, Hudgins L (2010) Array-based technology and recommendations for utilization in medical genetics practice for detection of chromosomal abnormalities. Genet Med 12: 742-745. doi: 10.1097/GIM.0b013e3181f8baad.
31. Miller D T, Adam M P, Aradhya S, Biesecker L G, Brothman A R, et al. (2010) Consensus Statement: Chromosomal Microarray Is a First-Tier Clinical Diagnostic Test for Individuals with Developmental Disabilities or Congenital Anomalies. Am J Hum Genet 86: 749-764. doi: 10.1016/j.ajhg.2010.04.006.
32. Glessner JT, Wang K, Cai G, Korvatska O, Kim C E, et al. (2009) Autism genome-wide copy number variation reveals ubiquitin and neuronal genes. Nature 459: 569-573. doi:10.1038/nature07953.
33. Qiao Y, Riendeau N, Koochek M, Liu X, Harvard C, et al. (2009) Phenomic determinants of genomic variation in autism spectrum disorders. J Med Genet 46: 680-688. doi:10.1136/jmg.2009.066795.
34. Wang K, Li M, Hadley D, Liu R, Glessner J, et al. (2007) PennCNV: An integrated hidden Markov model designed for high-resolution copy number variation detection in whole-genome SNP genotyping data. Genome Res 17: 1665-1674.
   doi:10.1101/gr.6861907.
35. Kent W J, Sugnet C W, Furey T S, Roskin K M, Pringle T H, et al. (2002) The human genome browser at UCSC. Genome Res 12: 996-1006. doi:10.1101/gr.229102.
36. Feng J, Schroer R, Yan J, Song W, Yang C, et al. (2006) High frequency of neurexin 1β signal peptide structural variants in patients with autism. Neurosci Lett 409: 10-13. doi:10.1016/j.neulet.2006.08.017.
37. Kim H-G, Kishikawa S, Higgins A W, Seong I-S, Donovan D J, et al. (2008) Disruption of Neurexin 1 Associated with Autism Spectrum Disorder. Am J Hum Genet 82: 199-207.
38. Ching M S L, Shen Y, Tan W-H, Jeste S S, Morrow E M, et al. (2010) Deletions of NRXN1 (neurexin-1) predispose to a wide spectrum of developmental disorders. Am J Med Genet B Neuropsychiatr Genet 153B: 937-947. doi:10.1002/ajmg.b.31063.
39. Schaaf C P, Boone P M, Sampath S, Williams C, Bader P I, et al. (2012) Phenotypic spectrum and genotype-phenotype correlations of NRXN1 exon deletions. Eur J Hum Genet. Available:http://dx.doi.org/10.1038/ejhg.2012.95.
40. Camacho-Garcia R J, Planelles M I, Margalef M, Pecero M L, Martinez-Leal R, et al. (2012) Mutations affecting synaptic levels of neurexin-1β in autism and mental retardation. Neurobiol Dis 47: 135-143. doi:10.1016/j.nbd.2012.03.031.
41. Wu Y-W, Prakash K, Rong T-Y, Li H-H, Xiao Q, et al. (2011) Lingo2 variants associated with essential tremor and Parkinson's disease. Hum Genet 129: 611-615. doi:10.1007/s00439-011-0955-3.
42. Yamamoto Y, Mochida S, Miyazaki N, Kawai K, Fujikura K, et al. (2010) Tomosyn Inhibits Synaptotagmin-1-mediated Step of Ca2+-dependent Neurotransmitter Release through Its N-terminal WD40 Repeats. J Biol Chem 285: 40943 -40955.
   doi:10.1074/jbc.M110.156893.
43. Williams A L, Bielopolski N, Meroz D, Lam A D, Passmore D R, et al. (2011) Structural and Functional Analysis of Tomosyn Identifies Domains Important in Exocytotic Regulation. J Biol Chem 286: 14542 -14553. doi:10.1074/jbc.M110.215624.
44. Hedges D, Hamilton-Nelson K, Sacharow S, Nations L, Beecham G, et al. (2012) Evidence of novel fine-scale structural variation at autism spectrum disorder candidate loci. Mol Autism 3:2. doi: 10.1186/2040-2392-3-2.
45. Nunn C, Mao H, Chidiac P, Albert P R (2006) RGS17/RGSZ2 and the RZ/A family of regulators of G-protein signaling. Semin Cell Dev Biol 17: 390-399.
   doi:10.1016/j.semcdb.2006.04.001.
46. Shema E, Kim J, Roeder R G, Oren M (2011) RNF20 inhibits TFIIS-facilitated transcriptional elongation to suppress pro-oncogenic gene expression. Mol Cell 42: 477-488. doi:10.1016/j.molce1.2011.03.011.
47. Carrie A, Jun L, Bienvenu T, Vinet M C, McDonell N, et al. (1999) A new member of the IL-1 receptor family highly expressed in hippocampus and involved in X-linked mental retardation. Nat Genet 23: 25-31. doi:10.1038/12623.
48. Gambino F, Pavlowsky A, Begle A, Dupont J-L, Bahi N, et al. (2007) IL1-receptor accessory protein-like 1 (IL1RAPL1), a protein involved in cognitive functions, regulates N-type Ca2+-channel and neurite elongation. Proc Natl Acad Sci USA 104: 9063-9068. doi:10.1073/pnas.0701133104.
49. Biswas A K, Johnson D G (2012) Transcriptional and nontranscriptional functions of E2F1 in response to DNA damage. Cancer Res 72: 13-17. doi:10.1158/0008-5472.CAN-11-2196.
50. Sumioka A, Imoto S, Martins R N, Kirino Y, Suzuki T (2003) XB51 isoforms mediate Alzheimer's beta-amyloid peptide production by X11 L (X11-like protein)-dependent and -independent mechanisms. Biochem J 374: 261-268.

### doi:10.1042/BJ20030489.

51. Stone T W, Forrest C M, Darlington L G (2012) Kynurenine pathway inhibition as a therapeutic strategy for neuroprotection. FEBS J 279: 1386-1397. doi:10.1111/j.1742-4658.2012.08487.x.
52. Sun J, Jayathilake K, Zhao Z, Meltzer H Y (n.d.) Investigating association of four gene regions (GABRB3, MAOB, PAH, and SLC6A4) with five symptoms in schizophrenia. Psychiatry Res.
   Available:http://www.sciencedirect.com/science/article/pii/S0165178111008195.
53. Yalçin Ö (2012) Genes and molecular mechanisms involved in the epileptogenesis of idiopathic absence epilepsies. Seizure 21: 79-86. doi:
   10.1016/j.seizure.2011.12.002.
54. Kirov G, Rujescu D, Ingason A, Collier D A, O'Donovan M C, et al. (2009) Neurexin 1 (NRXN1) Deletions in Schizophrenia. Schizophr Bull 35: 851-854.
   doi:10.1093/schbul/sbp079.
55. Harrison V, Connell L, Hayesmoore J, McParland J, Pike M G, et al. (2011) Compound heterozygous deletion of NRXN1 causing severe developmental delay with early onset epilepsy in two sisters. Am J Med Genet A. 155A: 2826-2831.
   doi:10.1002/ajmg.a.34255.
56. Kalia L V, Kalia S K, Chau H, Lozano A M, Hyman B T, et al. (2011) Ubiquitinylation of α-Synuclein by Carboxyl Terminus Hsp70-Interacting Protein (CHIP) Is Regulated by Bcl-2-Associated Athanogene 5 (BAGS). PLoS ONE 6: e14695.
   doi:10.1371/journal.pone.0014695.
57. Swaminathan S, Kim S, Shen L, Risacher S L, Foroud T (2011) Genomic Copy Number Analysis in Alzheimer's Disease and Mild Cognitive Impairment: An ADNI Study. Int J Alzheimers Dis 2011: 10. doi:10.4061/2011/729478.
58. Havik B, Le Hellard S, Rietschel M, LybæH, Djurovic S, et al. (2011) The Complement Control-Related Genes CSMD1 and CSMD2 Associate to Schizophrenia. Biol Psychiatry 70: 35-42. doi: 10.1016/j.biopsych.2011.01.030.
59. Vilarino-Guell C, Wider C, Ross O, Jasinska-Myga B, Kachergus J, et al. (2010) LINGO1 and LINGO2 variants are associated with essential tremor and Parkinson disease. Neurogenetics 11: 401-408. doi:10.1007/s10048-010-0241-x.
60. Punia S, Das M, Behari M, Mishra BK, Sahani A K, et al. (2010) Role of polymorphisms in dopamine synthesis and metabolism genes and association of DBH haplotypes with Parkinson's disease among North Indians. Pharmacogenet Genomics 20:435-441. doi:10.1097/FPC.0b013e32833ad3bb
61. Kao W-T, Wang Y, Kleinman J E, Lipska B K, Hyde T M, et al. (2010) Common genetic variation in Neuregulin 3 (NRG3) influences risk for schizophrenia and impacts NRG3 expression in human brain. Proc Natl Acad Sci U S A 107: 15619-15624. doi:10.1073/pnas.1005410107.
62. Grant S G (2012) Synaptopathies: diseases of the synaptome. Curr Opin Neurobiol 22:522-529.
   Available:http://www.sciencedirect.com/science/article/pii/S0959438812000244.
63. Michel M, Schmidt M J, Mimics K (2012) Immune system gene dysregulation in autism & schizophrenia. Dev Neurobiol.
   Available:http://www.ncbi.nlm.nih.gov/pubmed/22753382. Accessed 20 Jul. 2012.
64. Davis L K, Meyer K J, Rudd D S, Librant A L, Epping E A, et al. (2009) Novel copy number variants in children with autism and additional developmental anomalies. J Neurodev Disord 1: 292-301. doi:10.1007/s11689-009-9013-z.
65. Kang J-Q, Barnes G (n.d.) A Common Susceptibility Factor of Both Autism and Epilepsy: Functional Deficiency of GABA_{A} Receptors. J Autism Dev Disord: 1-12.
   doi:10.1007/s10803-012-1543-7.
66. Hogart A, Nagarajan RP, Patzel K A, Yasui D H, Lasalle J M (2007) 15q11-13 GABAA receptor genes are normally biallelically expressed in brain yet are subject to epigenetic dysregulation in autism-spectrum disorders. Hum Mol Genet 16: 691-703. doi:10.1093/hmg/ddm014.
67. Cook E H Jr, Lindgren V, Leventhal B L, Courchesne R, Lincoln A, et al. (1997) Autism or atypical autism in maternally but not paternally derived proximal 15q duplication. Am J Hum Genet 60: 928-934.
68. Xu L, Li Y, Zhang X, Sun H, Sun D, et al. (2011) Deletion of LCE3C and LCE3B genes is associated with psoriasis in a northern Chinese population. Br J Dermatol 165: 882-887. doi:10.1111/j.1365-2133.2011.10485.x.
69. Bergboer J G M, Zeeuwen P L J M, Schalkwijk J (2012) Genetics of Psoriasis: Evidence for Epistatic Interaction between Skin Barrier Abnormalities and Immune Deviation. The J Invest Dermatol.
   Available:http://www.ncbi.nlm.nih.gov/pubmed/22622420. Accessed 20 Jul. 2012.
70. Prescott S M, Lalouel J M, Leppert M (2008) From Linkage Maps to Quantitative Trait Loci: The History and Science of the Utah Genetic Reference Project. Annu Rev Genom Human Genet 9: 347-358. doi:10.1146/annurev.genom.9.081307.164441.
71. Price A L, Patterson N J, Plenge R M, Weinblatt M E, Shadick N A, et al. (2006) Principal components analysis corrects for stratification in genome-wide association studies. Nat Genet 38: 904-909. doi:10.1038/ng1847.
72. Huang D W, Sherman B T, Lempicki R A (2008) Systematic and integrative analysis of large gene lists using DAVID bioinformatics resources. Nat Protocols 4: 44-57. doi:10.1038/nprot.2008.211.
73. Huang D W, Sherman B T, Lempicki R A (2009) Bioinformatics enrichment tools: paths toward the comprehensive functional analysis of large gene lists. Nucleic Acids Res 37: 1-13. doi:10.1093/nar/gkn923.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A diagnostic test for diagnosing or predicting ASD in a subject comprising: a reagent for detecting at least one CNV genetic marker associated with ASD, wherein the at least one CNV genetic marker associated with ASD comprises
at least one CNV genetic marker associated with ASD listed in Table 3
wherein the reagent for detecting comprises one or more sets of oligonucleotides that specifically hybridize to a CNV genetic marker associated with ASD, the one or more oligonucleotides comprising DNA probes selected from the sequences set forth in SEQ ID NOs: 7410-7426; 27988-28001; 32494-32587; 62966-62998; and 69319-69561; and
wherein detection in a genetic sample from the subject of the at least one CNV genetic marker associated with ASD indicates that the subject is affected with ASD, or is predisposed to ASD.

2. The diagnostic test of claim 1, wherein the at least one CNV genetic marker associated with ASD listed in Table 3 is selected from the group consisting of the CNV genetic markers associated with ASD numbered 4-7, 9-12, 14-20 and 22-24 listed in Table 3.

3. The diagnostic test of claim 1, wherein the at least one CNV genetic marker associated with ASD listed in Table 3 is selected from the group consisting of the CNV genetic markers associated with ASD numbered 1-20 and 22-24 listed in Table 3.

4. The diagnostic test of claim 63, wherein the at least one CNV genetic marker associated with ASD listed in Table 3 comprises one or more of the CNV genetic markers numbered 6, 8, 10, 16 and 22 in Table 3 and wherein the -one or more CNV genetic markers associated with ASD listed in Table 4 comprises one or more of CNV genetic markers numbered 2-5, 8-10, 16, 20, 22, 24, 30 and 32 listed in Table 4.

5. The diagnostic test of claim 63, wherein the at least one CNV genetic marker associated with ASD listed in Table 3 comprises one or more of the CNV genetic markers numbered 2, 8, 11-13, 21 and 24 listed in Table 3; and the one or more CNV genetic markers associated with ASD listed in Table 4 comprises one or more of CNV genetic markers numbered 4, 6, 7, 10, 18, 19, 21, 22, 23, 26, 29 and 30 listed in Table 4.

6-9. (canceled)

10. The diagnostic test of claim 1, wherein the ASD in the subject comprises autism, Asperger's disorder, pervasive developmental disorder not otherwise specified, or childhood disintegrative disorder.

11.-12. (canceled)

13. The diagnostic test of claim 1, wherein the one or more sets of oligonucleotides each comprises from about 10 to about 25 oligonucleotides.

14.-15. (canceled)

16. The diagnostic test of claim 1, wherein the one or more sets of oligonucleotides are on an array.

17-19. (canceled)

20. The diagnostic test of claim 1, wherein the one or more sets of oligonucleotides comprise amplification primers that amplify the CNV genetic marker associated with ASD.

21. (canceled)

22. A method of diagnosing or predicting ASD in a subject, comprising: detecting in a genetic sample isolated from the subject at least one CNV genetic marker associated with ASD listed in Table 3; thereby diagnosing or predicting ASD in the subject.

23. The method of claim 22, wherein the ASD in the subject comprises autism, Asperger's disorder, pervasive developmental disorder not otherwise specified, or childhood disintegrative disorder.

24. The method of claim 22, wherein the at least one CNV genetic marker associated with ASD listed in Table 3 is selected from the group consisting of the CNV genetic markers associated with ASD 1-20 and 22-24 listed in Table 3.

25. The method of claim 64 wherein the at least one CNV genetic marker associated with ASD listed in Table 3 comprises one or more of the CNV genetic markers numbered 6, 8, 10, 16 and 22 in Table 3 and wherein the one or more CNV genetic markers associated with ASD listed in Table 4 comprises one or more of CNV genetic markers numbered 2-5, 8-10, 16, 20, 22, 24, 30 and 32 listed in Table 4.

26. The method of claim 64, wherein the at least one CNV genetic marker associated with ASD listed in Table 3 comprises one or more of the CNV genetic markers numbered 2, 8, 11-13, 21 and 24 listed in Table 3; and the one or more CNV genetic markers associated with ASD listed in Table 4 comprises one or more of the CNV genetic markers numbered 4, 6, 7, 10, 18, 19, 21, 22, 23, 26, 29 and 30 listed in Table 4.

27-30. (canceled)

31. The method of claim 64, wherein the at least one CNV genetic marker associated with ASD is detected by hybridizing one or more sets of DNA probes to at least one CNV genetic marker associated with ASD using a microarray, and wherein the one or more sets of DNA probes on the microarray comprise DNA probes selected from the sequences set forth in SEQ ID NOs:1-83,443.

32-51. (canceled)

52. A DNA microarray for detecting the presence of a CNV associated with ASD in a subject comprising one or more of the DNA probe sets selected from those set forth in SEQ ID NOs: 7410-7426; 12508-12563; 27988-28001; 31283-31314; 32494-32587; 33402-39860; 51803-52100; 61165-61290; 62966-62998; 64149-64167; 69319-69561.

53. The DNA microarray of claim 52 comprising at least 100 DNA probes selected from the DNA probes set forth in SEQ ID NOs: 7410-7426; 12508-12563; 27988-28001; 31283-31314; 32494-32587; 33402-39860; 51803-52100; 61165-61290; 62966-62998; 64149-64167; 69319-69561.

54.-62. (canceled)

63. The diagnostic test of claim 1 further comprising a reagent for detecting at least one CNV genetic marker associated with ASD, wherein the at least one CNV genetic marker associated with ASD comprises one or more CNV genetic markers associated with ASD listed in Table 4.

64. The method of claim 22 further comprising detecting in a genetic sample isolated from the subject at least one CNV genetic marker associated with ASD, wherein the at least one CNV genetic marker associated with ASD comprises one or more CNV genetic markers associated with ASD listed in Table 4.

65. The diagnostic test of claim 63 wherein the at least one CNV genetic marker associated with ASD comprises one or more CNV genetic markers associated with ASD listed in Table 4 wherein the reagent for detecting comprises one or more sets of oligonucleotides that specifically hybridizes to a CNV genetic marker associated with ASD, the one or more oligonucleotide comprising DNA probes selected from the sequences set forth in SEQ ID NOs: 12508-12563; 31283-31314; 33402-39860; 51803-52100; 61165-61290; and 64149-64167.
